SEQUENCE LISTING (52 sequences)

SEQ ID NO: 1 - protein, 9 aa, synthetic construct
  REGION 1..9: note = Circular
CRVLRSGSC

SEQ ID NO: 2 - protein, 8 aa, synthetic construct
GGSGGSKG

SEQ ID NO: 3 - DNA, 22 bp, synthetic construct
  source 1..22: mol_type = other DNA
gctgtggaacagaagagtgacc

SEQ ID NO: 4 - DNA, 23 bp, synthetic construct
  source 1..23: mol_type = other DNA
cccactcaacaagcgtgaatagc

SEQ ID NO: 5 - DNA, 23 bp, synthetic construct
  source 1..23: mol_type = other DNA
atgatggcttattacagtggcaa

SEQ ID NO: 6 - DNA, 20 bp, synthetic construct
  source 1..20: mol_type = other DNA
gtcggagattcgtagctgga

SEQ ID NO: 7 - DNA, 22 bp, synthetic construct
  source 1..22: mol_type = other DNA
cctctctctaatcagccctctg

SEQ ID NO: 8 - DNA, 21 bp, synthetic construct
  source 1..21: mol_type = other DNA
gaggacctgggagtagatgag

SEQ ID NO: 9 - DNA, 23 bp, synthetic construct
  source 1..23: mol_type = other DNA
actcacctcttcagaacgaattg

SEQ ID NO: 10 - DNA, 23 bp, synthetic construct
  source 1..23: mol_type = other DNA
ccatctttggaaggttcaggttg

SEQ ID NO: 11 - DNA, 20 bp, synthetic construct
  source 1..20: mol_type = other DNA
gatctcagtgcagaggctcg

SEQ ID NO: 12 - DNA, 20 bp, synthetic construct
  source 1..20: mol_type = other DNA
tttgcttgtccaggtggtcc

SEQ ID NO: 13 - DNA, 22 bp, synthetic construct
  source 1..22: mol_type = other DNA
agcgagtggatgccgcctttaa

SEQ ID NO: 14 - DNA, 22 bp, synthetic construct
  source 1..22: mol_type = other DNA
cattccaggcatctgcgatgag

SEQ ID NO: 15 - DNA, 22 bp, synthetic construct
  source 1..22: mol_type = other DNA
cactcacagacctgactcggtt

SEQ ID NO: 16 - DNA, 22 bp, synthetic construct
  source 1..22: mol_type = other DNA
aagcaggatcacagttggctgg

SEQ ID NO: 17 - DNA, 22 bp, synthetic construct
  source 1..22: mol_type = other DNA
gccactactgtgcctttgagtc

SEQ ID NO: 18 - DNA, 22 bp, synthetic construct
  source 1..22: mol_type = other DNA
ccctcagagaatcgccagtact

SEQ ID NO: 19 - DNA, 23 bp, synthetic construct
  source 1..23: mol_type = other DNA
ccttgatgccattaccagtctcc

SEQ ID NO: 20 - DNA, 22 bp, synthetic construct
  source 1..22: mol_type = other DNA
aaacagctccgcatcaacctgc

SEQ ID NO: 21 - DNA, 22 bp, synthetic construct
  source 1..22: mol_type = other DNA
ggagagtgtctgcggatacttc

SEQ ID NO: 22 - DNA, 23 bp, synthetic construct
  source 1..23: mol_type = other DNA
gcaggtagtgatgtgcaagagtc

SEQ ID NO: 23 - DNA, 22 bp, synthetic construct
  source 1..22: mol_type = other DNA
accctctgtgacttcatcgtgc

SEQ ID NO: 24 - DNA, 22 bp, synthetic construct
  source 1..22: mol_type = other DNA
ggagatgtagcacgggatcatg

SEQ ID NO: 25 - DNA, 20 bp, synthetic construct
  source 1..20: mol_type = other DNA
attgaaatcagccagcacgc

SEQ ID NO: 26 - DNA, 21 bp, synthetic construct
  source 1..21: mol_type = other DNA
aggaaccacagtgccagatcc

SEQ ID NO: 27 - DNA, 21 bp, synthetic construct
  source 1..21: mol_type = other DNA
ccttccaggatgaggacatga

SEQ ID NO: 28 - DNA, 21 bp, synthetic construct
  source 1..21: mol_type = other DNA
tgagtcacagaggatgggctc

SEQ ID NO: 29 - DNA, 22 bp, synthetic construct
  source 1..22: mol_type = other DNA
gagaaagtcaacctcctctctg

SEQ ID NO: 30 - DNA, 22 bp, synthetic construct
  source 1..22: mol_type = other DNA
gaagactcctcccaggtatatg

SEQ ID NO: 31 - DNA, 23 bp, synthetic construct
  source 1..23: mol_type = other DNA
ccagaaaccgctatgaagttcct

SEQ ID NO: 32 - DNA, 20 bp, synthetic construct
  source 1..20: mol_type = other DNA
caccagcatcagtcccaaga

SEQ ID NO: 33 - DNA, 22 bp, synthetic construct
  source 1..22: mol_type = other DNA
gagacagggaagtctgaagcac

SEQ ID NO: 34 - DNA, 22 bp, synthetic construct
  source 1..22: mol_type = other DNA
ccagcagtagttgctcctcttc

SEQ ID NO: 35 - DNA, 23 bp, synthetic construct
  source 1..23: mol_type = other DNA
gacagcctgtgttcgaggatatg

SEQ ID NO: 36 - DNA, 23 bp, synthetic construct
  source 1..23: mol_type = other DNA
tgttcttacaggagagggtagac

SEQ ID NO: 37 - DNA, 19 bp, synthetic construct
  source 1..19: mol_type = other DNA
gttggctcagccagatgca

SEQ ID NO: 38 - DNA, 24 bp, synthetic construct
  source 1..24: mol_type = other DNA
agcctactcattgggatcatcttg

SEQ ID NO: 39 - DNA, 22 bp, synthetic construct
  source 1..22: mol_type = other DNA
cacacttcccaggaatcaagcc

SEQ ID NO: 40 - DNA, 22 bp, synthetic construct
  source 1..22: mol_type = other DNA
tttggtgacacgacggaacagg

SEQ ID NO: 41 - DNA, 22 bp, synthetic construct
  source 1..22: mol_type = other DNA
tcttggttccctggcgtactct

SEQ ID NO: 42 - DNA, 23 bp, synthetic construct
  source 1..23: mol_type = other DNA
gtgagtgtcactctccagtttgc

SEQ ID NO: 43 - DNA, 24 bp, synthetic construct
  source 1..24: mol_type = other DNA
gaagaacaatccagactagcagca

SEQ ID NO: 44 - DNA, 25 bp, synthetic construct
  source 1..25: mol_type = other DNA
ccttatagggaacttcacatcacag

SEQ ID NO: 45 - DNA, 18 bp, synthetic construct
  source 1..18: mol_type = other DNA
aaacggaagcgcatcgaa

SEQ ID NO: 46 - DNA, 20 bp, synthetic construct
  source 1..20: mol_type = other DNA
gggactggcgagccttagtt

SEQ ID NO: 47 - DNA, 19 bp, synthetic construct
  source 1..19: mol_type = other DNA
caacctggacgccatcaag

SEQ ID NO: 48 - DNA, 21 bp, synthetic construct
  source 1..21: mol_type = other DNA
cagacggctgagtagggaaca

SEQ ID NO: 49 - protein, 9 aa, synthetic construct
  REGION 1..9: note = Circular
CGNKRTRGC

SEQ ID NO: 50 - protein, 9 aa, synthetic construct
  REGION 1..9: note = Circular
CRGDKGPDC

SEQ ID NO: 51 - protein, 9 aa, synthetic construct
  REGION 1..9: note = Circular
CRNGRGPDC

SEQ ID NO: 52 - protein, 9 aa, synthetic construct
  REGION 1..9: note = Circular
  SITE 1..9: note = all amino acids in the sequence are D-amino acids
CRVLRSGSC

(19) United States
(12) Patent Application Publication
(10) Pub. No.: US 2025/0352654 A1
(43) Pub. Date: Nov. 20, 2025

(54) PHARMACEUTICAL AGENT CONJUGATES TO MODULATE MACROPHAGE AND INFLAMMATORY FUNCTIONS AND USES THEREOF

(71) Applicant: REGENTS OF THE UNIVERSITY OF MINNESOTA, MINNEAPOLIS, MN (US)

(72) Inventors: Hongbo PANG, MINNEAPOLIS, MN (US); Jibin GUAN, MINNEAPOLIS, MN (US); Xian WU, MINNEAPOLIS, MN (US); Hong GUO, MINNEAPOLIS, MN (US)

(21) Appl. No.: 18/866,119

(22) PCT Filed: May 18, 2023
(86) PCT No.: PCT/US2023/067173

§ 371 Date: Nov. 15, 2024

Related U.S. Application Data
(60) Provisional application No. 63/343,189, filed on May 18, 2022.

(51) Int. Cl. A61K 47/64; A61K 31/573; A61K 47/55; A61K 47/60; A61K 47/69; A61P 11/00; A61P 19/02

(52) U.S. Cl. CPC A61K 47/64; A61K 31/573; A61K 47/55; A61K 47/60; A61K 47/6903; A61P 11/00; A61P 19/02

(57) ABSTRACT

The present invention provides a means to target pharmaceutical agents to sites of inflammation within the body using a conjugate that includes the pharmaceutical agent linked by a cleavable linker to a polymer. The conjugate may also include a homing molecule, such as the targeting peptide CRV, linked to the polymer via a second linker. Specifically, the present invention provides the conjugates and methods of using these conjugates to reduce inflammation and treat inflammatory diseases.

### CROSS-REFERENCE TO RELATED APPLICATIONS

This application claims the benefit of and priority to U.S. Application No. 63/343,189 filed on May 18, 2022, the content of which is incorporated herein by reference in its entirety

### STATEMENT REGARDING FEDERALLY SPONSORED RESEARCH

This invention was made with government support under CA214550 and GM133885 awarded by the National Institutes of Health. The government has certain rights in the invention.

### SEQUENCE LISTING

A Sequence Listing accompanies this application and is submitted as an ASCII file of the sequence listing named “920171_00529.xml” which is 48,919 bytes in size and was created on May 17, 2023. The sequence listing is electronically submitted via EFS-Web with the application and is incorporated herein by reference in its entirety.

### BACKGROUND

Steroid drugs and retinoic acid are widely used to manage acute and chronic inflammatory diseases. However, these drugs suppress immune functions and cause serious side effects. These side effects limit the utilization of these drugs, especially for long-term treatment of chronic inflammation. Accordingly, there remains a need in the art for a means to specifically target anti-inflammatory drugs to the inflamed disease site, which would serve to both increase drug activity at the disease site and to reduce side effects in healthy organs.

### SUMMARY

In a first aspect, the present invention provides conjugates including a pharmaceutical agent linked to a polymer. The pharmaceutical agent is linked by a cleavable linker to the polymer. The polymer may have a molecular weight between about 300 Da and about 20 kDa.

In some aspects, the conjugate may further include a peptide linked to the polymer via a second linker. The peptide may allow for targeting or delivery of the pharmaceutical agent to a particular tissue, organ or site within the body after administration. In some embodiments, the peptide is or comprises SEQ ID NO: 1, SEQ ID NO: 49, SEQ ID NO: 50, or SEQ ID NO: 51. In some embodiments, the peptide comprises an antibody or antigen binding fragment. In some embodiments, the conjugate may further comprise a biomolecule linked via a second linker to the polymer.

In another aspect, the present invention provides pharmaceutical compositions comprising the conjugate described herein and a pharmaceutically acceptable carrier.

In another aspect, the present invention provides methods for reducing inflammation in a subject. The methods comprise administering the conjugate or pharmaceutical composition described herein to the subject.

In another aspect, the present invention provides methods for treating an inflammatory disease in a subject. The methods comprise administering the conjugate or pharmaceutical composition described herein to the subject.

### BRIEF DESCRIPTION OF THE DRAWINGS

FIGS. 1A-1H demonstrate that CRV (SEQ ID NO: 1) selectively targets the myeloid cells in inflamed tissues due to the elevated RXRB expression. (A) Representative immunohistochemistry (IHC) images of targeting RXRB by CRV peptide in the lung of acute lung injury (ALI) mice. Control (PBS) or ALI (LPS-treated) mice were intravenously injected with FAM-labeled CRV or a control peptide (GGS) for 1 h homing, respectively. The lungs were collected for IHC analysis on whole-cell RXRB expression (left) and peptide amount (detected by anti-FITC antibody, right). (B) Quantification of FITC expression for FAM-labeled peptides in the lungs by IHC staining. (C) Representative immunofluorescence (IF) images of colocalization of rabbit anti-RXRB antibody with CD11b in PBS controls (top) and acute lung injury (ALI) mice (bottom). The lungs were collected for IF staining for the indicated markers after 6 h in vivo homing by intravenous injection of 25 mg of rabbit polyclonal RXRB antibodies in 100 mL of PBS in mice. The mice receiving intratracheal injection of PBS served as healthy controls. All the animal experiments were performed in three mice per group. (D) Relative percentage of RXRB-positive cells in different tissues of ALI mice. The indicated tissues were collected from the mice with ALI, dissociated into live single cells, and stained with different antibodies before fixation and flow-cytometry analysis. The healthy mice served as the controls. The inflammatory cells were stained with surface CD45, CD64, and RXRB. The percentage of RXRB+ cells (CD45+CD64+ cells) was normalized to that of healthy control as fold changes and plotted on the y axis. All the animal experiments were performed in 5-8 mice per group and repeated three times. (E) Fractions of indicated cells in total leukocytes of ALI lung (left) and percentage of RXRB+ portions in each immune subset of ALI lung after 24 h and 4 days of LPS injection (right). The myeloid cells were selected by plotting CD45+ and CD11b+ cells. The neutrophils were identified as Ly6G+ cells. The macrophages were further selected from non-neutrophils (Ly6G-) with F4/80+ cells. The healthy mice served as the controls. All the animal experiments were performed in 5-8 mice per group and repeated three times. (F) Preferential binding of FAM-CRV to immune cells by flow cytometry. In vivo binding study of FAM-CRV to cells was performed by intravenous injection of FAM-CRV or FAM-GGS control peptide in ALI mice for 1 h homing. The isolated cells from ALI lung were stained with surface CD45, Ly6G, and F4/80 for different immune cells, respectively. The bound CRV or GGS on the cell surface was stained by anti-FITC antibody for FAM. Left: mean fluorescent intensity (MFI) of FAM+ cells in each immune subset. Right: flow-cytometry histogram of CRV binding to the immune cells. The fluorescence intensity for CRV or GGS binding per cells was plotted in the histograms. The cells stained with secondary antibody only served as negative controls. (G) Representative IHC images of RXRB, CD64, and CD11b staining in the lung from human subjects with interstitial lung diseases (ILD). (H) Quantification of RXRB in IHC sections from lungs of human subjects with ILD. The samples from human subjects were performed in four normal lung tissues and four from patients with ILD, respectively. FAM, carboxyfluorescein; FITC, fluorescein iso-thiocyanate; A.U., arbitrary units; MFI, mean fluorescence intensity; Neg, negative. Data shown are mean±SD. Student's t test was performed for statistical analysis. *p<0.05, **p<0.01, ***p<0.001 versus control or healthy group. Scale bar, 100 mm.

FIGS. 1I-1P demonstrate in vivo CRV peptide distribution and its colocalization with macrophages in acute lung injury model. (I) Whole tissue scans of immunohistochemistry (IHC) images of peptide accumulation in the lung stained with anti-FITC antibody for FAM (arrows). Control (PBS) or ALI (LPS-treated) mice were intravenously injected with FAM-labeled CRV or a control peptide (GGS) for 1 h homing, respectively. (J) Representative IHC images of peptide accumulation in the lung stained with anti-FITC antibody for two other controls, FAM-labeled sCRV and FAM-Cys, upon intravenous injection for 1 h, respectively. Scale bars: 100 μm. Representative immunofluorescence (IF) images of the co-localization of FAM-labeled peptide with CD11b in the tissues of ALI mice after 1 h homing by intravenous injection of FAM-CRV or FAM-GGS (K-O), as well as FAM-sCRV or FAM-Cys (P). The sections were stained with anti-FITC antibody for FAM (green), anti-CD11b for macrophages (red), and Hoechst for nuclei (blue). Scale bars: 150 μm. The mice receiving intratracheal injection of PBS served as healthy controls. All the animal experiments were performed in 3 mice per group. FAM, carboxyfluorescein; FITC, fluorescein isothiocyanate; sCRV, scrambled CRV.

FIGS. 1Q-1T demonstrate in vivo anti-RXRB antibody homing in acute lung injury model. (Q-T) Representative IF images of the co-localization of rabbit polyclonal anti-RXRB IgG antibody and CD11b in the tissues of healthy (PBS control) and ALI mice (LPS) after 6 h homing by intravenous injection of 25 μg anti-RXRB antibody or normal rabbit IgG as the control antibody. The sections were stained with anti-rabbit IgG secondary antibody for RXRB antibody and control IgG (green), anti-CD11b for macrophages (red), and Hoechst for nuclei (blue). Scale bars: 150 μm. The mice receiving intratracheal injection of PBS served as healthy controls. All the animal experiments were performed in 3 mice per group.

FIGS. 1U-1AA demonstrate surface expression of RXRB on inflammatory cells of ALI lung via flow cytometry gating. The cells were incubated with the indicated primary antibodies and rabbit anti-RXRB, or rabbit IgG as a negative control at 4° C. for 1 h and analyzed by flow cytometry. The gating was started with choosing the live cells by plotting SSC-A vs. FSC-A. Single cells were then selected by plotting FSC-A vs. FSC-H on the live cells followed by subpopulation of CD45+ cells (leukocytes). (U) Percentage of inflammatory cells in different tissues of healthy control and ALI mice by staining surface CD45 and CD64. Percentage of RXRB+ portions in immune subsets of ALI lung. (V) Fractions of leukocytes in total cells from indicated tissues (left) and percentage of RXRB+ leukocytes of different tissues (right). (W) Flow cytometry gating strategy for neutrophils and pulmonary macrophages. The myeloid cells were selected by plotting CD45+ and CD11b+ cells. The neutrophils were identified as Ly6G+ cells. The macrophages were further selected from non-neutrophils (Ly6G-) with F4/80+ cells. The histogram compares the intensity of RXRB in neutrophils and macrophages, respectively. (X) Flow cytometry gating strategy for lymphocytes and myeloid cells in the lung of ALI mice. The lymphocytes were selected by plotting CD45+ cells and SSC-A. The myeloid cells were selected by plotting CD45+ and CD64+ cells. The histograms compare the intensity of RXRB in lymphocytes and myeloid cells. (Y) Flow cytometry gating strategy for alveolar (CD11b-F4/80+CD11C+) and interstitial macrophages (CD11b+F4/80+CD11C−). The histogram compares the intensity of RXRB in alveolar and interstitial macrophages of ALI lung, respectively. (Z) The content of CRV in different immune subsets of ALI lungs after 1 h intravenous injection as evaluated by percentage of FAM+ cells (AA) and mean fluorescence intensity (MFI) of FAM+ cells. GGS served as the control peptide. **P<0.01, ***P<0.001 vs control group.

FIGS. 1AB-1AD demonstrate RXRB colocalization with inflammatory cells in the lung tissues from human subjects with interstitial lung disease. (AB) Quantification of CD64, and (AC) CD11b in IHC sections from lungs of human subjects with ILD. (AD) Representative IF images of co-localization of RXRB with CD64 in the lung from healthy and interstitial lung diseases (ILD). The sections were stained with anti-RXRB antibody RXRB (green), anti-CD64 for inflammatory cells (red), and Hoechst for nuclei (blue). Scale bars: 150 m. The normal lung tissues served as healthy controls. The samples from human subjects were performed in 4 normal lung tissues and 4 from patients with ILD, respectively. **P<0.01 vs healthy group.

FIG. 2A-2H demonstrate synthesis and in vitro characterization of CRV-prednisolone conjugate with reactive oxygen species responsive linker (A) Chemical structure of CRV-prednisolone (CRV-PSL) conjugate showing the drug payload PSL, reactive oxygen species (ROS)-responsive linker, PEG2000, maleimide, cysteine, Ahx linker, FAM, and CRV. PSL, prednisolone; PEG2000, polyethylene glycol 2000; Cys, cysteine; Ahx, 6-aminohexanoic acid; FAM, fluorescein amidites. (B) Chromatograms by HPLC showing absorption peaks of CRV-PSL after incubation with mouse plasma with inflammation for 0 h and 24 h, respectively. The plasma was obtained from the mice receiving intraperitoneal injection of 0.3 mg kg⁻¹ LPS for 4 h. (C-F) Gene expression of inflammatory cytokines in LPS-stimulated RAW 264.7 macrophages by CRV-PSL conjugate. CRV-PSL or free PSL was added to the cells 1 h before LPS stimulation (pre-PSL) or 1 h after LPS stimulation (post-PSL). The effect of CRV-PSL on inflammatory cytokines in LPS-stimulated RAW cells was examined after 18 h of drug treatment. The cells receiving PBS alone served as the control group. mRNA expression of inflammatory markers of IL-1b (C), iNOS (D), IL-6 (E), and MCP-1 (F) was quantified and normalized by that of PBS control group (y axis). The experiment was repeated three times and the data shown are mean±SEM. Student's t test was performed for statistical analysis. **p<0.01, ***p<0.001 versus PBS control; ###p<0.001 versus LPS group. (G-H) Protein expression of IL-1b and iNOS in LPS-stimulated RAW 264.7 macrophages by CRV-PSL treatment. GAPDH and tubulin, respectively served as internal controls of the target proteins. The experiment was repeated three times and the data shown are mean±SD. *p<0.05, **p<0.01, ***p<0.001. There is no statistical difference between any other groups. A.U., arbitrary units; IL-1b, interleukin 1b; MCP-1, monocyte chemoattractant protein-1; iNOS, inducible nitric oxide synthase; GAPDH, glyceraldehyde-3-phosphate dehydrogenase.

FIGS. 2I-2L discloses synthesis and structure of (I) PSL-PEG-Cys-FAM-CRV (FAM-CRV-PSL) and (J) PSL-PEG-Cys-FAM (FAM-Cys-PSL). (K) 1H NMR spectrum of PSL-PEG-Cys-FAM-CRV (500 MHz, DMSO): δ 8.58 (s, 1H), 8.01 (s, 4H), 7.55-7.19 (m, 6H), 7.03 (d, J=20.8 Hz, 5H), 6.70 (d, J=2.3 Hz, 1H), 6.63-6.53 (m, 2H), 6.16 (dd, J=10.1, 1.9 Hz, 2H), 5.92 (d, J=1.9 Hz, 2H), 5.18 (s, 3H), 4.76-4.21 (m, 12H), 4.08 (d, J=18.6 Hz, 4H), 3.83-2.94 (m, 194H), 2.89-1.93 (m, 74H), 1.89-1.81 (m, 2H), 1.69-0.59 (m, 39H). (L) 1H NMR spectrum of PSL-PEG-Cys-FAM (500 MHz, CDCl3): δ 7.28 (d, J=7.3 Hz, 27H), 6.72 (d, J=17.7 Hz, 5H), 6.45 (s, 3H), 6.02 (s, 2H), 4.49 (s, 3H), 3.94 (t, J=7.0 Hz, 2H), 3.84 (t, J=7.2 Hz, 3H), 3.64 (s, 194H), 3.42 (q, J=5.3 Hz, 4H), 3.02 (t, J=7.0 Hz, 3H), 2.86-2.69 (m, 7H), 2.52 (t, J=7.2 Hz, 7H), 2.35 (s, 4H), 1.69 (s, 39H), 1.46 (s, 4H), 1.39 (t, J=7.2 Hz, 8H), 1.32-1.18 (m, 41H), 0.98 (s, 7H), 0.91-0.77 (m, 22H). NMR: nuclear magnetic resonance; PSL: prednisolone; PEG2000: polyethylene glycol 2000; Cys: cysteine; Ahx: 6-aminohexanoic acid. FAM: fluorescein amidites; HO-PEG2000-MAL: maleimide-PEG2000-Hydroxyl; DCC: 2,2′-Thiodiacetic acid, N, N′-Dicyclohexylcarbodiimide; DMAP: 4-dimethylaminopyridine.

FIGS. 2M-2N disclose in vitro verification of intracellular cleavage of FAM-CRV-PSL (M) Illustration of ROS-sensitive drug release mechanism of CRV-PSL triggered by ROS. (N) Absorption peaks of CRV-PSL in the presence of 10 mM H2O2 monitored by high-performance liquid chromatography (HPLC) at a wavelength of 285 nm. Left: CRV-PSL or PSL dissolved in PBS (irresponsive prodrug) served as control. Peaks with an elution time of 8.6 min represent CRV-PSL, while peaks at 9.2 min represent PSL. Middle: Chromatograms obtained for the reaction mixture after incubation of CRV-PSL with 10 mM H2O2 at 37° C. for 5 min, 24 h, and 72 h, respectively. Right: In vitro drug release profile of CRV-PSL with different concentrations of H2O2 at 37° C. for 24 h. The PSL release from CRV-PSL conjugate without H2O2 treatment served as the control group. ***P<0.001 between groups.

FIGS. 2O-2S disclose an inhibitory effect of CRV-prednisolone (CRV-PSL) conjugate on inflammatory genes in macrophages in vitro. (O-P) Gene expression of transforming growth factor beta 1 (TGFβ) 1 and collagen type I, alpha 1 (Col1a1) in LPS-stimulated RAW 264.7 macrophages by CRV-PSL conjugate. CRV-PSL or free PSL was added to the cells 1 h before LPS stimulation (pre-PSL) or 1 h after LPS stimulation (post-PSL). The effect of CRV-PSL on inflammatory cytokines in LPS-stimulated RAW cells were examined after 18 h of drug treatment, respectively. mRNA expression was quantified and normalized by that of PBS control group (y-axis). (Q-S) Gene expression of inflammatory cytokines in LPS-stimulated THP-1 macrophages by CRV-PSL conjugate. After the differentiation of THP-1 cells to macrophages by 100 ng/ml of phorbol-12-myristate-13-acetate (PMA) for 24 h, CRV-PSL or free PSL (1 μM) was added to the cells with LPS stimulation (100 ng/ml). The effect of CRV-PSL on inflammatory cytokines in LPS-stimulated THP-1 cells were examined after 18 h of treatment, respectively. mRNA expression of inflammatory markers of tumor necrosis factor alpha (TNFα) (Q), monocyte chemoattractant protein-1 (MCP1) (R), and interleukin 6 (IL6) (S) was quantified and normalized by that of PBS control group (y-axis). The experiment was repeated three times and the data shown are mean±standard error of the mean (SEM). Student's t-test was performed for statistical analysis. ***P<0.001 vs control; #P<0.05, ###P<0.001 vs LPS group. && P<0.01 vs free PSL group.

FIG. 2T discloses detection of intracellular content of PSL metabolites. Top: Structure of prednisolone and its metabolites, 20-α-OH-PSL and 20-β-OH-PSL. Bottom: Measurement of PSL metabolites, 20-α-OH-PSL and 20-β-OH-PSL, by liquid chromatography coupled to tandem mass spectrometry (LC/MS) in RAW 264.7 cells treated with 10 μM of free PSL or CRV-PSL for 4 h. LC/MS chromatograms show the area comparison of daughter ion at m/z 345 (20-ca-OH-PSL and 20-β-OH-PSL) from parent ion at m/z 363 of free PSL and CRV-PSL group. PSL and CRV-PSL controls were directly injected into the LC/MS system, serving as the controls of treatment groups, respectively. PSL: prednisolone; PEG2000: polyethylene glycol 2000; Cys: cysteine; Ahx: 6-aminohexanoic acid. FAM: fluorescein amidites; HO-PEG2000-MAL: maleimide-PEG2000-Hydroxyl; DCC: 2, 2′-Thiodiacetic acid, N, N′-Dicyclohexylcarbodiimide; DMAP: 4-dimethylaminopyridine; A.U., arbitrary unit; m/z: mass-to-charge ratio.

FIG. 2U discloses mean plasma concentration-time curves of PSL following intravenous administration of free PSL, Cys-PSL and CRV-PSL with an equivalent PSL dosage at 1 mg kg⁻¹. All the animal experiments were performed in 3-4 mice per group. The data are presented as the mean±standard deviation (SD). Statistical significance was calculated using Student's t test. *P<0.05, **P<0.01, CRV-PSL vs free PSL group; #P<0.05, ##P<0.01, ###P<0.001 Cys-PSL vs free PSL group.

FIGS. 3A-3B disclose issue biodistribution and targeting of CRV-PSL conjugate in vivo. (A) Representative IF images of the colocalization of FAM-labeled peptide with F4/80 in the lung of PBS controls and ALI mice. The lungs were collected for IF staining for the indicated markers after 1 h of intravenous injection of FAM-Cys-PSL and FAM-CRV-PSL, with a 1 mg kg⁻¹ equivalent dose of PSL, respectively. (B) Tissue distribution of free PSL in ALI mice by liquid chromatography coupled to tandem mass spectrometry (LC/MS). After 4 days of intratracheal LPS challenge, the mice were treated with free PSL, Cys-PSL, and CRV-PSL, respectively. The dose was equivalent of 1 mg kg⁻¹ of PSL in weight. The tissues were collected after 2 h of treatment. All the animal experiments were performed in 3-4 mice per group. Data are presented as mean±SD. Statistical significance was calculated using Student's t test. *p<0.05, **p<0.01. There is no statistical difference between any other groups. ND, not determined. Scale bar, 150 mm.

FIGS. 3C-3F demonstrate representative IF images of the co-localization of FAM-labeled peptide with macrophage marker F4/80 in the tissues of PBS controls and ALI mice. The tissues were collected for IF staining for the indicated markers after 1 h of intravenous injection of FAM-Cys-PSL and FAM-CRV-PSL, with 1 mg kg⁻¹ equivalent dosage of PSL, respectively. The sections were stained with anti-FITC antibody for FAM (green), anti-F4/80 for macrophages (red), and Hoechst for nuclei (blue). All the animal experiments were performed in 3-4 mice per group. Scale bar: 150 μm.

FIGS. 4A-F demonstrates CRV conjugation increasing the therapeutic efficacy of PSL against lung injury (A) Diagram of treatment timeline in ALI mice. (B) Representative hematoxylin-eosin (H&E)-stained lung sections subjected to histopathological analyses. Left: lung of healthy (PBS) and ALI mouse with no treatment. Right: lung of ALI mouse treated with free PSL, Cys-PSL, and CRV-PSL at a dose equivalent of 0.01 mg kg⁻¹ (top) and 0.1 mg kg⁻¹ (bottom) of PSL. (C) Quantification of inflammatory response in the lungs following H&E staining. (D) Percentage of CD64+ cells. Quantification of (E) myeloperoxidase (MPO) and (F) iNOS expression in the lungs by IHC staining. The mice were given the free PSL, Cys-PSL, and CRV-PSL at a dose equivalent of 0.01 mg kg⁻¹ or 0.1 mg kg⁻¹ of PSL intravenously every 2 days for 4 days after intratracheal LPS challenge, and the tissues were collected on day 4 for histopathological analyses. All experiments were performed in 3-4 mice per group. Data are presented as mean±SD. Statistical significance was calculated using Student's t test. *p<0.05, **p<0.01, ***p<0.001; ns, not significant. A.U., arbitrary units. Scale bar, 100 mm.

FIGS. 4G-4I demonstrate therapeutic efficacy of CRV-PSL conjugate in ALI mice. (G) Representative images of CD64, (H) iNOS, and (I) myeloperoxidase (MPO) staining by IHC in lungs of ALI mice. Top: Lung of healthy (PBS) and ALI mouse with no treatment. Middle: Lung of ALI mouse treated with free PSL, Cys-PSL, and CRV-PSL with a dose equivalent of 0.01 mg kg⁻¹ of PSL. Bottom: Lung of ALI mouse treated with free PSL, Cys-PSL, and CRV-PSL with a dose equivalent of 0.1 mg kg⁻¹ of PSL. The mice were given the free PSL, Cys-PSL, and CRV-PSL with different doses as indicated intravenously every 2 days for 4 days after intratracheal LPS challenge and the tissues were collected on day 4 for histopathological analyses. All experiments were performed in 3-4 mice per group. Scale bar: 100 μm.

FIGS. 5A-5F demonstrate that CRV conjugation reduces the acute side effects of PSL in healthy organs (A) Thymus weight in mice receiving 0.1 mg kg⁻¹ or 1 mg kg⁻¹ equivalent dose of PSL by free PSL and CRV-PSL treatment, respectively. (B) Quantification of immunofluorescent signals of TUNEL-positive cells showing apoptotic cells in tissues of mice receiving different treatment. (C and D) Flow-cytometry analysis of blood cell populations in mice with different treatment. The count of (C) blood lymphocytes and (D) T cells in mice receiving different treatment were normalized to the vehicle group. (E and F) Plasma levels of liver enzymes (E) alanine transaminase (ALT) and (F) aspartate transaminase (AST). The healthy mice were given free PSL or CRV-PSL conjugate every 2 days for 5 days with an equivalent dose of PSL at 0.1 mg kg⁻¹ and 1 mg kg⁻¹, respectively. The weight of thymus was measured, and the blood was collected for flow cytometry 4 h after the third dose of treatment. All experiments were performed in 3-4 mice per group, and the experiment was repeated three times. Data shown are mean±SD. Student's t test was performed for statistical analysis. *p<0.05, **p<0.01, ***p<0.001. There is no statistical difference between any other groups.

FIGS. 5G-5H demonstrate toxicity and biological safety of CRV-PSL in vivo. (G) IF images of TUNEL assay showing apoptotic cells in the thymus and other tissues of mice. The healthy mice were given the treatment of free PSL or CRV-PSL conjugate every 2 days for 5 days with an equivalent dose of PSL at 0.1 mg kg⁻¹, and 1 mg kg⁻¹, respectively. (H) Flow cytometry gating strategy for lymphocytes, T cells, and their subpopulations in the blood from CRV-PSL treated mice. The gating was started with choosing the live cells by plotting SSC-A vs. FSC-A. Single cells were then selected by plotting FSC-A vs. FSC-H for the live cells followed by subpopulation of CD45+ cells. The lymphocytes were selected by plotting CD45+ cells and SSC-A, which were then selected for T cells by CD3 staining. The tissues and the blood were collected 4 hours after the third dose of treatment. All experiments were performed in 3-4 mice per group and the experiment was repeated three times. The data are presented as the mean±standard deviation (SD). Statistical significance was calculated using Student's t test. Scale bars: 150 m.

FIGS. 6A-6B show that CRV accumulates in arthritic joints. (A) The CRV receptor RXRB is expressed at a significantly higher level (indicated by brown IHC signal) in arthritic joints of K/Bx arthritic mice (bottom) than in the joints of healthy mice (top). (B) The arthritic mice were injected intravenously with a control peptide (GGS, top) or CRV (bottom). The mice were sacrificed after 1 hour and joints were collected for IHC staining. CRV showed much higher accumulation in arthritic joints than the control peptide.

FIG. 7A illustrates synthesis and structure of DEX-PEG-Cys-FAM-CRV (FAM-CRV-DEX). DEX: dexamethasone; PEG2000: polyethylene glycol 2000; Cys: cysteine; FAM: fluorescein amidites; HO-PEG2000-MAL: maleimide-PEG2000-Hydroxyl; DCC: 2, 2′-Thiodiacetic acid, N, N′-Dicyclohexylcarbodiimide; DMAP: 4-dimethylaminopyridine.

FIGS. 7B-7D illustrates In vivo CRV peptide distribution in arthritic mouse models. (B) Scheme of synthesis of 64Cu-DOTA-CRV. (C) Representative immunohistochemistry (IHC) images of peptide accumulation in the tissues of collagen-induced-arthritis (CIA) mice with anti-FITC antibody for FAM-labeled CRV and FAM-GGS, upon intravenous injection for 1 h, respectively. Scale bars: 100 μm. (D) Representative IHC images of CRV-peptide and a control peptide (GGS) accumulation in the ankle joints of K/B×N serum-transfer arthritic mice at 1 h post intravenous injection as detected by FAM signals with anti-FITC antibody. Scale bar: 200 μm for 10× magnification; 100 μm for 20× magnification. All the animal experiments were performed in 3 mice per group. DOTA, 1,4,7,10-tetraazacyclododocane tetraacetic acid; FAM, carboxyfluorescein; FITC, fluorescein isothiocyanate.

FIG. 8A-8G CRV targets the inflamed synovium of the arthritic joints. (A) Scheme of the establishment of collagen-induced arthritis (CIA) model. (B) In vivo PET/CT images of 64Cu-DOTA and 64Cu-DOTA-CRV accumulation at the joints of mice with CIA. 64Cu-DOTA or 64Cu-DOTA-CRV were intravenously injected into CIA mice followed by PET/CT scanning at 1 h, 3 h, and 24 h post-injection. Healthy mice receiving 64Cu-DOTA-CRV served as controls. (C) Quantification of 64Cu-DOTA or 64Cu-DOTA-CRV uptake at the knees and ankles by analyzing PET data of the region of interest (ROI). The experiments were performed on three mice in each group. All data represent mean value±standard error (SD). Student's t-test was performed for statistical analysis. *P<0.05 between groups. (D) Representative 3D PET/CT images of 64Cu-DOTA and 64Cu-DOTA-CRV biodistribution from a 3 h scan of mice after intravenous injection. Dashed circles indicate the accumulation of 64Cu-DOTA-CRV at the joints. (E-F) CRV homing in the arthritic knee joints of CIA mice by immunohistochemistry (IHC) and immunofluorescence (IF) analysis. CIA mice were intravenously injected with FAM-labeled CRV or a control peptide (GGS) for 1 h homing, respectively. (E) Representative IHC images of CRV-peptide accumulation in the knee joint as detected by FAM signals with anti-FITC antibody. Scale bar: 100 μm. (F) Representative IF images of co-localization of FAM-labeled peptides with CD64 in CIA mice as stained with the anti-FITC antibody for FAM (green), the anti-CD64 antibody for inflammatory cell marker CD64 (red), and Hoechst for nuclei (blue). Scale bar: 150 μm. All the animal experiments were performed on three mice per group. (G) Preferential binding of FAM-CRV to immune cells by flow cytometry. In vitro binding study of FAM-CRV to cells was performed by incubation with 10 μM of FAM-CRV or a FAM-GGS control peptide in the isolated cells from CIA joints for 1 h at 4□C. The isolated cells were stained with surface CD45, Ly6G, and F4/80 for leukocytes (CD45+), neutrophils (CD45+Ly6G+), and macrophages (CD45+Ly6G-F4/80+), respectively. The bound CRV or GGS on cell surface was stained by an anti-FITC antibody for FAM. The cells stained with secondary antibodies only served as negative controls. The data shown are mean±standard error (SD). Student's t-test was performed for statistical analysis. *P<0.05 vs GGS control. FAM, carboxyfluorescein; FITC, fluorescein isothiocyanate. DOTA, 1,4,7,10-tetraazacyclododocane tetraacetic acid.

FIGS. 8H-8I show flow cytometry analysis of the surface expression of RXRB on inflammatory cells in the arthritic joints of CIA mice. The cells were isolated and incubated with the indicated primary antibodies and rabbit anti-RXRB, or rabbit IgG as a negative control at 4 □C for 1 h and analyzed by flow cytometry. The gating started with choosing the live cells by plotting SSC-A vs. FSC-A. Single cells were then selected by plotting FSC-A vs. FSC-H on the live cells followed by subpopulations of CD45+ cells (leukocytes), Ly6G+(neutrophils), and F4/80+(macrophages). (H) Percentage of inflammatory cells in joint tissues of healthy control and CIA mice by staining surface CD45 (left) and the portion of inflammatory cell subpopulations in the leukocytes (right). The experiments were performed on three mice in each group. All data represent mean value±standard error (SD). Student's t-test was performed for statistical analysis. *** P<0.001 between groups. (I) Flow cytometry gating strategy for neutrophils, macrophages, and lymphocytes. The leukocytes were selected by plotting CD45+. The neutrophils were identified as Ly6G+ cells. The macrophages were further selected from non-neutrophils (Ly6G-) with F4/80+ cells. The lymphocytes were selected by plotting CD45+ cells and SSC-A.

FIG. 9A-9F show elevated RXRB expression in the inflamed synovial tissues of arthritic joints (A) Left: Representative hematoxylin and eosin (H&E) staining of inflamed synovial tissues (pannus) with increased infiltrate in the knee joints of collagen-induced arthritis (CIA) mice. Middle and right: immunohistochemistry (IHC) images of RXRB and inflammatory marker CD64 expression at the pannus of the knee in CIA mice. Scale bar: 200 μm for 10× magnification; 100 μm for 20× magnification. (B) Representative H&E images of pannus formation and cartilage and bone erosion (arrows) at ankle joints, and IHC images of RXRB and its colocalization with CD64 at ankle joints of CIA mice. Scale bar: 200 μm. (C) Top: Representative H&E images of pannus formation and damaged cartilage and bone at ankle joints, and IHC images of RXRB and CD64 expression at ankle joints of mice with K/B×N serum-transfer arthritis. Scale bar: 200 μm for 10× magnification; 100 μm for 20× magnification. Bottom: Arthritic mice were injected intravenously with a control peptide GGS or CRV. (D) The gene expression of RXRB in synovial tissues of human subjects with rheumatoid arthritis (RA). RXRB mRNA expression was quantified and normalized by that of healthy control group (y-axis). The samples from human subjects were performed in 3 normal synovial tissues and 3 from patients with RA, respectively. The data shown are mean±standard error of the mean (SEM). Student's t-test was performed for statistical analysis. ***P<0.001 between two groups. (E-F) Flow cytometry analysis of the percentage of RXRB-positive cells and mean fluorescence intensity (MFI) of RXRB in the different immune subsets from inflamed joints of CIA mice. The healthy mice served as the controls. The isolated cells from arthritic joints were stained with surface CD45, Ly6G, and F4/80 for different immune cells. All the animal experiments were performed in 3 mice per group and have been repeated three times. The data shown are mean±standard error (SD). Student's t-test was performed for statistical analysis. *P<0.05, **P<0.01, ***P<0.001 vs healthy group. A.U., arbitrary unit.

FIG. 9G-9H. Gene expression in human synovial tissues of rheumatoid arthritis (RA). The gene expression of inflammatory markers (G) and those related to extracellular matrix (ECM) formation (H) in the synovial tissues of human subjects with RA. mRNA expression was quantified and normalized by that of the healthy control group (y-axis). The experiments were performed on three subjects in each group. The experiment was repeated three times and the data shown are mean±standard error of the mean (SEM). Student's t-test was performed for statistical analysis. ***P<0.001 between groups.

FIGS. 10A-10B show synthesis in vitro characterization of CRV-dexamethasone (CRV-DEX) conjugate with reactive oxygen species (ROS) responsive linker. (A) Chemical structure of CRV-DEX conjugate showing the drug payload ROS-responsive linker, PEG2000, maleimide, cysteine, Ahx linker, FAM, and CRV. DEX: dexamethasone; PEG2000: polyethylene glycol 2000; Cys: cysteine; Ahx: 6-aminohexanoic acid; FAM: fluorescein amidites; ROS: reactive oxygen species; DCC: N, N′-Dicyclohexylcarbodiimide; DMAP: 4-Dimethylaminopyridine. (B) The gene expression of inflammatory cytokines in LPS-stimulated RAW 264.7 macrophages by CRV-DEX conjugate was determined. The cells were incubated with CRV-DEX or free DEX with or without LPS stimulation for 18 h. The cells receiving PBS alone served as the control group. mRNA expression of inflammatory markers and those related to extracellular matrix formation was quantified and normalized by that of the PBS control group (y-axis). The experiment was repeated three times and the data shown are mean±standard error of the mean (SEM). Student's t-test was performed for statistical analysis. *P<0.05, **P<0.01, ***P<0.001 vs PBS control; ##P<0.01, ###P<0.001 vs LPS group. IL1β: interleukin 1 beta; IL18: interleukin 18; IL6: interleukin 6; MCP-1: monocyte chemoattractant protein-1; TNF□: tumor necrosis factor-alpha; iNOS: inducible nitric oxide synthase; MMP: Matrix metalloproteinase; TIMP: tissue inhibitor of metalloproteinases. TIMP1: TIMP Metallopeptidase Inhibitor 1.

FIGS. 11A-11B illustrate pharmacokinetics and tissue biodistribution of CRV-DEX conjugate in vivo. (A) Mean plasma concentration-time curves of DEX following intravenous administration of free DEX, Cys-DEX and CRV-DEX with an equivalent DEX dosage at 1 mg kg-1. All the animal experiments were performed on 3 mice per group. The data are presented as the mean±standard deviation (SD). (B) Tissue distribution of free DEX in CIA mice by liquid chromatography coupled to tandem mass spectrometry (LC/MS). Once arthritis developed, the mice were given intravenous injections with free DEX, Cys-DEX, and CRV-DEX, respectively. The dose was equivalent of 1 mg kg⁻¹ of DEX in weight. The tissues were collected after 3 h of treatment. All the animal experiments were performed on 3 mice per group. The data are presented as the mean±standard deviation (SD). Statistical significance was calculated using Student's t test. *P<0.05, **P<0.01 between groups. There is no statistical difference between any other groups. ND, not determined.

FIGS. 12A-12C show CRV conjugation increases the therapeutic efficacy against arthritis in CIA mice. (A) Scheme of the treatment of collagen-induced arthritis (CIA) model. (B) CRV-DEX ameliorates the severity of arthritis in CIA mice. Left: Representative images of the hind paws of CIA mice by the end of the treatment. Right: Arthritis scores (top) and paw swelling levels as measured by paw thickness (bottom). The mice with arthritis were given intravenous injections of free DEX, Cys-DEX, and CRV-DEX with a dose equivalent of 0.01 mg kg⁻¹ of DEX dissolved in PBS/ethanol (0.03% v/v) once every two days for one week, n=4-6 per group. The data are presented as the mean±standard deviation (SD). Statistical significance was calculated using Student's t test. *P<0.05, **P<0.01, ***P<0.001, vs vehicle-treated CIA control mice. There is no statistical difference between any other groups. (C) Histology analysis of joint tissues in CIA mice by CRV-DEX treatment. Top: Representative hematoxylin-eosin (H&E) and Safranin O/Fast Green (Safranin O) stained knee sections subjected to histopathological analyses. Bottom: The quantification of the inflammatory response, proteoglycan loss, cartilage, and bone erosion in the arthritic joints following H&E and Safranin O staining. The mice were given the free DEX, Cys-DEX, and CRV-DEX with a dose equivalent of 0.01 mg kg⁻¹ intravenously every two days for 7 days. The tissues were collected on day 10 for histopathological analyses. All experiments were performed in 4-6 mice per group. The data are presented as the mean±standard deviation (SD). Statistical significance was calculated using Student's t test. *P<0.05, **P<0.01, *** P<0.001; Scale bar: 200 μm.

FIGS. 13A-13C show CRV conjugation alleviates arthritis by enhancing the anti-inflammation effects in arthritic joints of CIA mice. (A) Gene expression of inflammatory cytokines in arthritic joints of CIA mice by CRV-DEX conjugate. After the development of arthritis, the CIA mice were given intravenous injections of free DEX, Cys-DEX, and CRV-DEX with a dose equivalent of 0.01 mg kg⁻¹ of DEX once every two days for one week. The mice receiving vehicles alone served as the control group. mRNA expression of inflammatory markers of IL13, IL6, MCP1, iNOS, MMP12, and MMPP13 was quantified and normalized by that of the vehicle control group (y-axis). The experiment was repeated three times and the data shown are mean±standard error of the mean (SEM). Student's t-test was performed for statistical analysis. * P<0.05, **P<0.01, ***P<0.001 between groups. There is no statistical difference between any other groups. (B) Representative images and (C) quantification of CD64, iNOS, MMP9, and RXRB expression, in the knee joints by IHC staining. Red stars indicate the signals of the target protein and inflammatory cell infiltration at the pannus. The mice were given the free DEX, Cys-DEX, and CRV-DEX at a dose equivalent of 0.01 mg kg⁻¹ intravenously every two days for one week after the development of arthritis. The tissues were collected on day 10 for histopathological analyses. All experiments were performed in 4-6 mice per group. The data are presented as the mean±standard deviation (SD). Statistical significance was calculated using Student's t test. *P<0.05, **P<0.01, ***P<0.001 between groups. There is no statistical difference between any other groups. Scale bar: 200 μm. IL13: interleukin 1 beta; IL6: interleukin 6; MCP-1: monocyte chemoattractant protein-1; iNOS: inducible nitric oxide synthase; MMP: Matrix metalloproteinase. A.U., arbitrary unit.

FIGS. 14A-14G show the therapeutic effect of CRV-dexamethasone (CRV-DEX) conjugate in rat CIA model. (A) Scheme of the establishment and treatment strategy of collagen-induced arthritis (CIA) in rat. (B) Hind paw images and micro-CT scans of the rats treated with free dexamethasone (DEX) and CRV-DEX, with a dose equivalent of 0.02 mg kg⁻¹ of DEX intravenously as indicated in the treatment scheme. White arrows in hind paw images indicate the difference in paw thickness in different groups; the yellow arrows in micro-CT images show bone erosion with reduced bond density in DEX-treated rats. (C) Body weight and (D) paw thickness in the rats receiving intravenous injections of DEX and CRV-DEX, with a dose equivalent of 0.02 mg kg⁻¹ of DEX every 2 days for 7 days in CIA rats. (E) Representative images of knee joints by hematoxylin-eosin (H&E) and Safranin O/Fast green (Safranin O) staining of CIA rats after the treatment as indicated above. Semi-quantification of cartilage damage by the score of proteoglycan loss (F) and bone damage by the bone erosion score (G) in Safranin O/Fast green staining sections. Scale bar: 200 μm. All experiments were performed in 3 rats per group. All data represent mean value±standard error (SD). Student's t-test was performed for statistical analysis. *P<0.05, *** P<0.001 between groups.

FIGS. 15A-15E show CRV conjugation reduces the acute and long-term toxicity of DEX in healthy organs. (A-C) The acute side effects of DEX in healthy organs of CIA mice. (A) Quantification of immunofluorescent signals (Top) and representative immunofluorescent images (bottom) of TUNEL-positive cells showing apoptotic cells in tissues of mice receiving short-term treatments. (B-C) Plasma levels of liver enzymes alanine transaminase (ALT) and (F) aspartate transaminase (AST). The CIA mice were given free DEX or CRV-DEX conjugate intravenously every two days for one week with an equivalent dose of DEX at 0.01 mg kg⁻¹, respectively. (D-E) The long-term toxicity of DEX in healthy organs. Thymus and spleen weight in mice receiving long-term treatments.

FIG. 16A shows that the D-isoform of CRV (D-CRV), but not the L-isoform of CRV (L-CRV), accumulates in tumors following oral administration. FAM-labeled L-CRV or D-CRV was orally administered to tumor-bearing mice. While little L-CRV accumulated in the tumor (top), a significant amount of D-CRV accumulated in the tumor (bottom).

FIG. 16B shows biodistribution of free DEX in RA mice model by liquid chromatography coupled to tandem mass spectrometry (LC/MS). Once arthritis developed, the mice were given subcutaneous injections with free DEX, and (D)-CRV-DEX loaded Pluronic F127-HA (HP) hydrogel, respectively. The dose was equivalent of 3 mg kg⁻¹ of DEX. Tissues were collected after 6 h of treatment. All the animal experiments were performed on 4 mice per group. Error bars indicate S.E.M., n=4. *p<0.05 and **p<0.01 (Student's t-test).

FIG. 17 shows chemical structures of CRV conjugated to dexamethasone (A), prednisolone (B and D), and all-trans retinoic acid (C). An ROS-responsive linker was utilized in the structures shown in A-C, whereas a non-cleavable linker was utilized in the structure shown in part D.

FIG. 18 shows a synthetic route of DEX-PEG conjugations with different PEG length. For 2k (PEG2000), 5k (PEG5000) and 10k (PEG10000) MW PEGs, n equals to 45, 113 and 227, respectively.

FIGS. 19A-19F shows the in vivo effects of DEX-PEG conjugations with different PEG lengths. (A) Body weight of mice in each treatment group. (B) Blood glucose of mice during the ITT experiment. Mice were fasted for 6 hours, and 7.5 U of insulin was administered via intraperitoneal (i.p.) injection. The blood glucose levels were measured using Clarity BG1000 Blood Glucose Meter Strips at 0, 15, 30, 60, 90, and 120 minutes after the injection. (C) Thymus weight of mice in each group at the end of the study. Triglycerides (D), total cholesterol (E) and non-HDL cholesterol (F) in mice plasma were quantified by LiquiColor® Test (Enzymatic) Kit. Error bars indicate S.E.M., n=4. *p<0.05 and **p<0.01 (Student's t-test).

FIG. 20 shows representative H&E images of mice liver after the treatment. Liver was collected, fixed and sectioned. H&E staining was carried out according to the standard protocol. Scale bar, 100 m for all images.

FIGS. 21A-21B shows the synthesis and effect of DEX=PEG conjugations with a pH sensitive linker. (A) Synthetic route of DEX-PEG conjugations with pH sensitive linker. (B) Cleavage test of pH sensitive DEX-PEG conjugation. 1 mg of conjugation was incubated in PBS (pH 5.0) and PBS (pH 7.0) for 12 h. DEX cleaved from the conjugation was separated using a spin column filter with 1000 molecular cutoff. The amount of DEX in wash trough was determined by HPLC. Error bars indicate S.E.M., n=3.

### DETAILED DESCRIPTION

The present invention provides a means to target pharmaceutical agents to sites of inflammation within the body by linking an anti-inflammatory agent to a polymer and optionally further linking this conjugate to a targeting peptide, such as the CRV peptide (SEQ ID NO: 1) used in the Examples. Specifically, the present invention provides conjugates and methods of using these conjugates to target pharmaceutical agents to particular tissues or cells within a subject. The conjugates provided herein may be targeted to particular cells or tissues to reduce inflammation and treat inflammatory diseases, such as acute lung injury (ALI) and rheumatoid arthritis (RA). Surprisingly the inventors also found that simply linking the pharmaceutical agent to a polymer using a cleavable linker allowed the drug to be targeted to tissues, delivered to cells and resulted in reduced inflammation. The pharmaceutical agent may be an anti-inflammatory, but targeted delivery of other pharmaceutical agents is contemplated herein. These delivery mechanisms may allow for higher doses of a pharmaceutical to be used to treat a subject due to a reduction in off-target effects of the pharmaceutical, but may also result in higher effectiveness of the same dose of the pharmaceutical due to more direct delivery after systemic administration of a pharmaceutical agent to the site or cells in need of the agent.

ALI is a life-threatening condition characterized by excessive and uncontrolled systemic inflammatory responses accompanied by extensive inflammatory cell infiltration, disruption of alveolar epithelial-endothelial capillary barrier, and destruction of alveolar structure, all of which finally lead to respiratory failure. ALI and its more severe form, acute respiratory distress syndrome (ARDS), are regarded as a major respiratory health threat and result in mortality in about 40% of patients in intensive care units worldwide. ALI may arise from various sources, including from pulmonary infection by bacteria or viruses, mechanical trauma, and chronic conditions such as asthma. RA is a chronic inflammatory autoimmune disease that affects about 1% of the population worldwide. It is characterized by an excessive inflammatory response at synovium with infiltration of immune cells and pannus formation, which further causes cartilage and bone destruction and finally leads to significant joint deformity, disability, and a reduction of life quality.

Because glucocorticoid drugs have potent anti-inflammatory activities, they were an obvious choice for ALI and RA treatment and have been tested in multiple clinical trials over the past several decades. Unfortunately, these trials have failed to show a clear benefit for these drugs in ALI/ARDS and RA treatment. One major factor limiting the clinical use of glucocorticoid drugs is their immune-related side effects, which are caused by nonspecific drug accumulation in healthy organs after systemic administration. The inventors hypothesized that delivery or selective accumulation of these pharmaceuticals to the sites of inflammation may lessen the side effects of these pharmaceuticals while allowing better treatment of the inflammation. While glucocorticoids are shown here, other anti-inflammatory drugs may be used in the invention.

Thus, to improve the utility of these drugs, the present inventors have developed a means to specifically target them to sites of inflammation within the body using a homing molecule, such as the targeting peptide CRV (SEQ ID NO: 1). For example, the inventors have previously demonstrated that the CRV peptide selectively homes to the infected lung in a murine model of bacterial lung infection following intravenous injection and that it colocalizes with macrophages in the infected lung. Additionally, they have demonstrated that CRV conjugation increases the amount of drug-carrying porous silicon nanoparticles (pSiNPs) that accumulate in the infected lung following intravenous injection, and that this improved delivery results in an increased efficacy for reducing infection-induced inflammation. The inventors have also developed a drug formulation for treatment of RA by conjugating dexamethasone (DEX) with a targeting peptide, CRV, to improve the selective biodistribution of DEX at the diseased site.

Macrophages and neutrophils account for the majority of immune infiltrates in ALI, and macrophage activation contributes to lung inflammation by triggering an innate immune response and promoting neutrophil infiltration. Thus, the inventors hypothesized that CRV, which was identified based on its ability to specifically bind to macrophages, could be used to facilitate drug delivery to the inflamed lung in ALI. Unfortunately, the CRV-pSiNP conjugates mentioned above were found to accumulate in the liver and spleen. Thus, to avoid the use of a bulky delivery agent, the inventors conjugated CRV directly to the drug prednisolone (PSL) to facilitate delivery. To function as a therapeutic, PSL needs to bind to its receptor in the cytosol and subsequently translocate into the nucleus. Thus, to facilitate release of this drug, a reactive oxygen species (ROS)-responsive linker was included between CRV and PSL within the conjugate. ROS are mainly found intracellularly and are generated in excess under inflammatory conditions. Thus, this design ensures that the CRV-PSL conjugate is stable in the circulatory system, and that PSL is only cleaved from CRV after entering an inflamed cell.

In Example 1, the inventors demonstrate (1) that CRV specifically targets the inflamed lung in a murine model of ALI, and (2) that the CRV-PSL conjugate described above exhibits increased therapeutic efficacy and an improved safety profile for the treatment of ALI as compared to free PSL. In Example 2, the inventors demonstrate that CRV can also be used to target the drug dexamethasone to inflamed joints to treat RA. In Example 3, the inventors demonstrate that, unlike the L-isoform of CRV, the D-isoform of CRV is effective when administered orally due to its increased stability. In example 4, the inventors demonstrate the synthesis of DEX-PEG conjugates with different linker lengths or cleavable linkers and the use of these conjugates in the absence of the CRV peptide. The inventors contemplate that other homing peptides could be used in place of the CRV peptide used in the Examples and that D-isoforms or mixed peptide containing both L- and D-amino acids may be useful in developing the conjugates described herein.

### Conjugates:

In a first aspect, the present invention provides conjugates comprising the formula: A-CL-P-H and A-CL-P. In this formula, A comprises a pharmaceutical agent, CL comprises a cleavable linker that links A to P and is cleaved after cellular uptake, P comprises a polymer with a molecular weight between about 300 Da and about 20 kDa that is optionally linked directly or via a second linker to H, and H is a homing molecule that increases the tissue and/or cellular selectivity for the conjugate. In some aspects, the homing molecule may include CRV, a circular peptide of SEQ ID NO: 1. While SEQ ID NO: 1 is used in the Examples other homing peptides or homing molecules are know in the art such as SEQ ID NO: 49, 50 or 51. A homing molecule could also be used instead and suitable homing biomolecules are known to those of skill in the art. Homing biomolecules may include folate, retinoic acid, retinoic acid derivatives, biotin, avidin, or galactose. Other homing molecules may include antibodies or antigen binding fragments thereof that allow the pharmaceutical agent to be targeted to a specific cell type or a condition associated with cells expressing a particular protein that can be targeted via the antibody.

As used herein, the term “conjugate” refers to a substance formed by combining two or more components. The conjugates of the present invention comprise at least three components, which are abbreviated herein as A, CL, and P. The conjugates may also comprise all four components and include A, CL, P, and H.

The first component of the conjugate, “A”, comprises a pharmaceutical agent (e.g., a drug). As used herein, the term “pharmaceutical agent” refers to a substance that has a physiological effect when introduced to the body of a subject. Any small molecule pharmaceutical agent may be used in the conjugates of the present invention. However, conjugation to H targets the drug to sites of inflammation in the body. Thus, in preferred embodiments, the pharmaceutical agent is an anti-inflammatory drug. Suitable anti-inflammatory drugs for use with the present invention include, without limitation, steroid drugs, retinoic acid, and methotrexate. In some embodiments, A is a glucocorticoid. Glucocorticoids are a class of corticosteroids that are widely used for the treatment of inflammation, allergies, autoimmune diseases, and cancers. To exert their therapeutic effects, glucocorticoids bind to the glucocorticoid receptor, which belongs to the nuclear receptor superfamily of transcription factors. In Example 1, the inventors demonstrate that conjugation of H (e.g., the cyclic peptide CRV) to the glucocorticoid prednisolone increases the efficacy of this pharmaceutical agent for the treatment of ALI. Thus, in some embodiments, A is prednisolone. In Example 2, the inventors demonstrate that conjugation of CRV to the synthetic glucocorticoid dexamethasone increases the efficacy of this pharmaceutical agent for the treatment of arthritis.

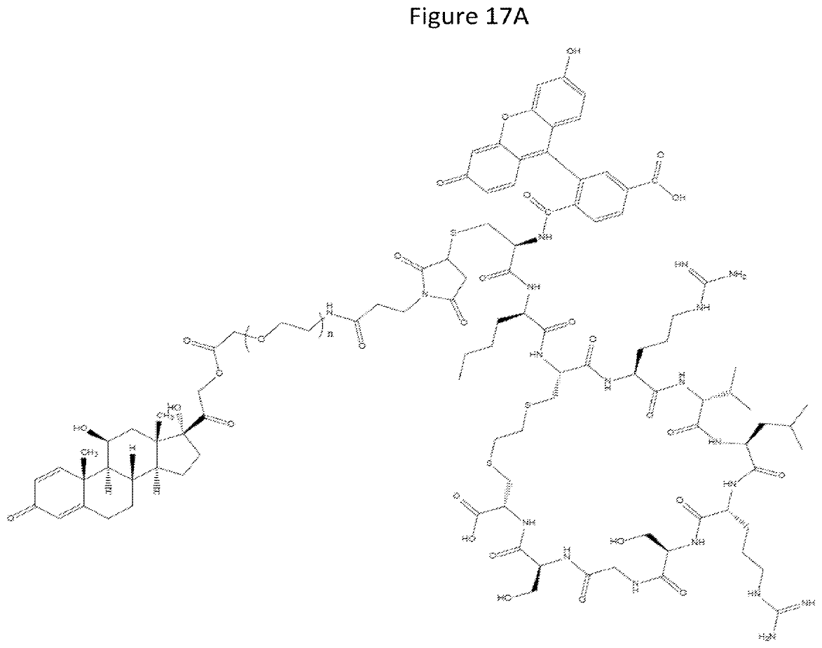

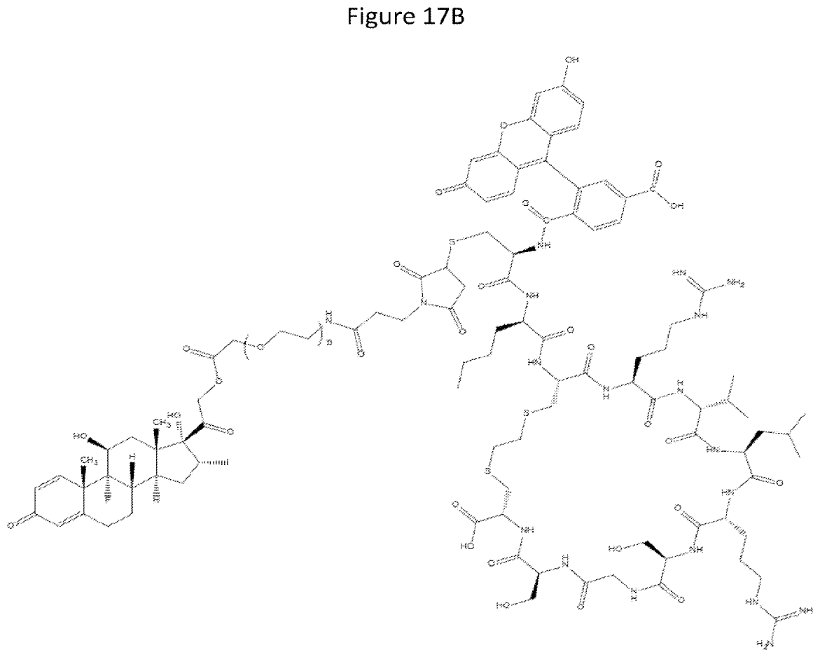

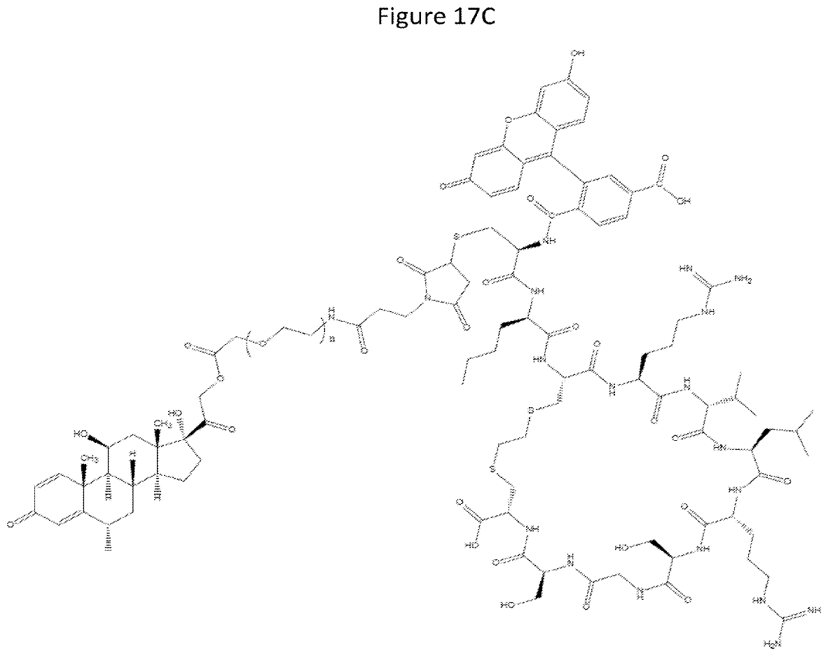

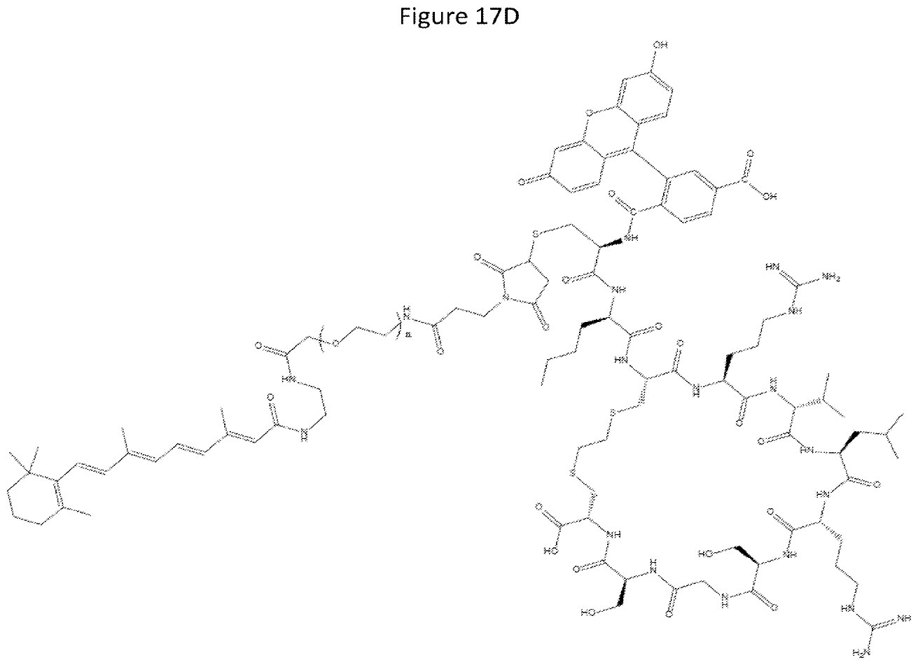

Thus, in some embodiments, A is dexamethasone. Exemplary structures showing how CRV can be conjugated to the pharmaceutical agent dexamethasone, prednisolone, and all-trans retinoic acid are provided in FIG. 17.

The second component of the conjugate, “CL”, comprises a cleavable linker. As used herein, the term “linker” refers to a moiety that links two components of a conjugate. The linkers used in the conjugates of the present invention are cleaved after cellular uptake, meaning that they are cleaved by a component or condition that is specifically found intracellularly. For example, many enzymes are found only intracellularly. Thus, in some embodiments, the cleavable linker is an enzyme-responsive linker. Examples of suitable intracellular enzymes include cathepsin, β-glucuronidase, and lysosomal enzymes. pH varies across different tissues, and inflammation can cause a drop in pH. Thus, in some embodiments, the cleavable linker is a pH-responsive linker. Examples of pH-responsive linkers include hydrazones and cis-aconityl. Inflammatory responses have been shown to cause cells to generate reactive oxygen species (ROS). Thus, in some embodiments, the cleavable linker is an ROS-responsive linker. Examples of suitable ROS-responsive linkers include thioacetic acid linkers. In the Examples, the inventors utilized 2,2′-thiodiacetic acid as an ROS-responsive linker. Thus, in specific embodiments, the cleavable linker is 2,2′-thiodiacetic acid. Importantly, the use of intracellularly cleaved linkers increases the specificity of agent delivery by ensuring that the pharmaceutical agent component of the conjugate (“A”) is only released after the conjugate has reached the target tissue.

Other examples of cleavable linkers include but are not limited to cleavable amine-reactive linkers, (e.g., N-hydroxysuccinimide ester (NHS) linkers, imidoester linkers, pentafluorophenyl ester linkers, and hydroxymethyl phosphine linkers), cleavable carboxyl-to-amine reactive linkers (e.g., carbodiimide linkers), cleavable sulfydryl-reactive cleavable linkers (e.g., malemide linkers, haloacetyl linkers, pyridyl disulfide linkers, thiosulfonate linkers, and vinyl sulfone linkers), cleavable aldehyde-reactive linkers (e.g., hydrazide linkers, alkoxyamine linkers), photoactive cleavable linkers (e.g., diazirine linkers, and aryl azide linkers), cleavable hydroxyl linkers (e.g., isocyanate linkers), and cleavable azide-reactive linkers (e.g., alkyne linkers and phosphine linkers). For instance, the conjugate may include an amine-reactive cleavable linker (e.g., one of many known commercial NHS linkers containing a disulfide moiety) that has conjugated an N-terminus of a peptide-based pharmaceutical agent to one of the other components of the conjugate.

Cleavable moieties, such as disulfide bridges, and cleavable/reversible biotinylation moieties may also be incorporated into the linkers listed herein and used to conjugate the pharmaceutical agent to other components of the conjugate.

The third component of the conjugate, “P”, comprises a polymer. A “polymer” is a substance that is composed of many repeating subunits. The polymers used in the conjugates of the present invention have a molecular weight between about 300 Da and about 20 kDa. Suitable molecular weights include, for example, 300 Da, 5 kDa, 10 kDa, and 20 kDa. In some embodiments, the polymer is polyethylene glycol (PEG). PEG is a polyether compound derived from petroleum. In the Examples, the inventors included PEG2000 in their conjugates because it is known to limit accumulation in the liver. Thus, in some embodiments, the polymer is PEG2000. Other suitable polymers for use in the conjugates of the present invention include hydrophilic polymers such as acrylic polymers (e.g., acrylic acid, acrylamide, and maleic anhydride polymers) and amine-functional polymers (e.g., allylamine, ethyleneimine, and oxazoline).

The polymer may be linked directly to the homing molecule, or it may be linked to homing molecule via a second linker. In the Examples, the inventors used a maleimide linkage to link the polymer to the N-terminal cysteine of CRV. Thus, in some embodiments, the second linker is a maleimide linker. Maleimide is a chemical compound with the formula H₂C₂(CO)₂NH that is an important building block in organic synthesis. However, any linker that does not interfere with the functions of the pharmaceutical agent and the homing molecule components of the conjugate can be utilized as a second linker. Other examples of suitable linkers include N-hydroxysuccinimide linkers and azide linkers.

In some aspects, the homing molecule includes the peptide, CRV of SEQ ID NO: 1. CRV was identified in a phage display screen for peptides that specifically bind to macrophages, as described in U.S. Patent Publication No. US20200190142, which is hereby incorporated by reference in its entirety. This peptide, which was named CRV based on its first three residues, comprises the sequence CRVLRSGSC (SEQ ID NO: 1). The two terminal cysteines of this sequence can form a disulfide bond, rendering this peptide cyclic. Thus, in some embodiments, CRV is a cyclic peptide.

All amino acids, except for glycine, exist as two stereoisomers. The “L-amino acid” is the stereoisomer whose amino group is on the left side in the Fisher projection, while the “D-amino acid” is the stereoisomer whose amino group is on the right side in the Fisher projection. L-amino acids are the form found in most proteins and are more susceptible to proteolytic cleavage than D-amino acids. In Example 3, the inventors demonstrate that, due to its increased stability, the D-isoform of CRV can be effectively administered orally (e.g., with an effective amount) whereas the L-isoform of CRV cannot. Thus, in some embodiments, CRV comprises the D-isoform of CRV (SEQ ID NO: 52). All homing molecules containing amino acids, including CRV, may include either or both D-amino acids and L-amino acids.

In another aspect, the homing molecule comprises a homing peptide. Homing peptides are oligopeptides, usually consisting of 30 or fewer amino acids that are efficiently and specifically bound to, taken up by and/or into cells. Examples of homing peptides that may be included in the conjugate include but are not limited to the cyclic peptide LyP-1 (SEQ. ID NO: 49; having a sequence of CGNKRTRGC), the cyclic peptide iRGD, (SEQ. ID NO: 50; having a sequence of CRGDKGPDC), and the cyclic peptide iNGR, (SEQ. ID NO: 51; having a sequence of CRNGRGPDC).

In another aspect, the homing molecule comprises an antibody, an antibody fragment (e.g., antigen-binding fragments (Fab), or single chain variable fragments (scFv)), or an antibody-like molecule, such as a nanobody. The antibody assists the conjugate in homing the conjugate to a specific cell or cellular environment. Examples of an antibody, an antibody fragment, or an antibody-like molecule that may be included in the conjugate include but are not limited to an anti-tumor necrosis factor alpha (TNFα) antibody, an anti-interleukin 1 beta (IL1β) antibody, an anti-interleukin 6 (IL6) antibody, an anti-monocyte chemoattractant protein-1 (MCP-1) antibody, an anti-matrix metalloproteinase 2 (MMP2) antibody, an anti-matrix metalloproteinase 3 (MMP3) antibody, an anti-matrix metalloproteinase 9 (MMP9) antibody, an anti-matrix metalloproteinase 13 (MMP13) antibody, an anti-tissue inhibitor of metalloproteinases 1 (TIMP1) antibody, an anti-inducible nitric oxide synthase (iNOS) antibody, or an anti-tissue inhibitor of metalloproteinases 2 (TIMP2) antibody.

In another aspect, the homing molecule comprises a biomolecule. A biomolecule includes any molecule known to interact or otherwise bind to other molecules (e.g., proteins) in living systems. Examples of biomolecules that may be included in the conjugate include but are not limited to folate, folic acid, retinoic acids and retinoic acid derivatives, avidin, and galactose.

In another aspect, the conjugate comprises linkers that conjugate the peptide, antibody, or biomolecule to the polymer or the pharmaceutical agent. In this regard, if two linkers are used in creating the conjugate, the linker conjugating the pharmaceutical agent to the polymer is referred to as a first linker and should be a cleavable linker, and the linker conjugating the polymer to the peptide, antibody, or biomolecule is referred to as a second linker. The second linker may be either cleavable or uncleavable, depending on the nature of the conjugate, and may include any linker chemistry disclosed herein or known to those of skill in the art. The conjugates may further comprise a detectable label so that the conjugate can be tracked within the body after administration of the conjugate to a subject. The detectable label can be an enzymatic or a fluorescent label. In one embodiment, the label is fluorescein.

### Pharmaceutical Compositions:

In a second aspect, the present invention provides pharmaceutical compositions comprising the conjugates described herein and a pharmaceutically acceptable carrier.

“Pharmaceutically acceptable carriers” are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media. The compositions of the present invention may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances or tonicity modifiers (e.g., lactose, mannitol). The conjugate may be administered in a hyrdrogel.

Mode of administration may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In the Examples, the inventors successfully delivered CRV conjugates to target tissues via intravenous, sub-cutaneous and oral administration. Thus, in some embodiments, the pharmaceutical composition is formulated for intravenous, sub-cutaneous or oral administration.

### Methods for Reducing Inflammation and Treating Inflammatory Diseases:

In a third aspect, the present invention provides methods for reducing inflammation in a subject. The methods comprise administering a therapeutically effective amount of the conjugates or pharmaceutical compositions described herein to the subject to reduce inflammation.

“Inflammation” is the immune system's natural response to injury, infection, and illness. A reduction in inflammation can be assessed by measuring the expression of inflammatory markers such as myeloperoxidase (MPO), IL1β, and inducible nitric oxide synthesis (iNOS). A reduction in inflammation can also be detected as a reduction in symptoms of inflammation such as swelling, redness, or pain, or as a reduction in a symptom of a specific inflammatory disease.

In a fourth aspect, the present invention provides methods for treating an inflammatory disease in a subject. The methods comprise administering a therapeutically effective amount of the conjugates or pharmaceutical composition described herein to the subject to treat the inflammatory disease in the subject.

“Inflammatory diseases” are diseases that are characterized by inflammation. CRV can be used to target a drug to any tissue that comprises inflammatory myeloid or lymphocytic cells. Thus, any inflammatory disease that results in tissue infiltration by inflammatory myeloid or lymphocytic cells can be treated using the methods of the present invention. Examples of such inflammatory diseases include acute lung injury (ALI), acute respiratory distress syndrome (ARDS), arthritis, lupus, eczema, chronic obstructive pulmonary disease (COPD), obesity, and infections (e.g., viral, bacterial, or fungal infections). In Example 1, the inventors demonstrate that conjugation of CRV to prednisolone increases the efficacy of this drug for the treatment of ALI. Thus, in some embodiments, the inflammatory disease is ALI and “A” is a glucocorticoid. In Example 2, the inventors demonstrate that conjugation of CRV to dexamethasone increases the efficacy of this drug for the treatment of arthritis. Thus, in some embodiments, the inflammatory disease is arthritis and “A” is a dexamethasone. The methods of the present invention reduce the risk for off-target effects, making anti-inflammatory drugs safer for long-term use. Thus, in some embodiments, the disease is a chronic disease that requires long-term treatment.

As used herein, “treating” describes the management and care of a subject for the purpose of combating a disease. Treating includes administering a conjugate or pharmaceutical composition of present invention to prevent the onset of the symptoms or complications, to alleviate the symptoms or complications, or to eliminate the disease. In the context of an inflammatory disease, treating may include a reduction of inflammation, swelling, pain, itching, fever, shortness of breath, stiffness, soreness, redness, loss of function of a body part.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. In the Examples, the inventors administrated the conjugates intravenously or orally. Thus, in some embodiments, the conjugate is administered intravenously or orally. In some embodiments, the conjugate or pharmaceutical composition is administered in multiple doses (e.g., an “effective dose” or “therapeutically effective amount) to the subject. For example, the conjugate or pharmaceutical composition may be administered in 2, 3, 4, 5, 6, 7, 8, or more doses. Administration can be continuous or intermittent.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

In the Examples, the inventors demonstrate that conjugation to CRV increases drug accumulation in inflamed tissues. Thus, in some embodiments, the conjugate accumulates in an inflamed tissue at higher levels than “A” administered alone. Increased accumulation may be any statistically significant increase in accumulation in affected tissues as compared to the pharmaceutical administered alone. In some embodiments, the increase in accumulation in affected tissues is 10% increase, 15% increase, 20%, 25% or even 30% more accumulation in the target tissue by the conjugate as compared to the agent alone. Further, the inventors demonstrated that inflamed tissues (i.e., the lungs of mice with ALI and the joints of mice with arthritis) express retinoid X receptor beta (RXRB) at increased levels. The inventors previously identified RXRB as the receptor for CRV and determined that elevated expression of RXRB in inflamed tissues is likely the basis for CRV targeting. Thus, in some embodiments, the inflamed tissue exhibits increased expression of RXRB. In some embodiments, the inflamed tissue is lung. In other embodiments, the inflamed tissue is a joint. However, the conjugates of the present invention may accumulate in any tissue that is inflamed. Additionally, in the Examples, the inventors demonstrate that conjugation to CRV reduces or does not significantly affect drug accumulation in healthy tissues. Thus, in some embodiments the conjugate accumulates in healthy tissues at lower or equivalent levels to “A” administered alone. Inflamed tissues can be distinguished from healthy tissues by the expression of inflammatory markers such as myeloperoxidase (MPO), IL1β, and inducible nitric oxide synthesis (iNOS). In some cases, inflamed tissues can also be distinguished visually, e.g., as being reddened, swollen, and/or hot.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure, and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument, and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: CRV-PSL for Treatment of Acute Lung Injury

Acute lung injury (ALI) is a life-threatening condition characterized by excessive and uncontrolled systemic inflammatory responses accompanied by extensive inflammatory cell infiltration, disruption of the alveolar epithelial-endothelial capillary barrier, and destruction of alveolar structure, all of which finally lead to respiratory failure. ALI and its more severe form, acute respiratory distress syndrome (ARDS), are regarded as a major health threat in the respiratory tract, which accounts for about 40% of mortality in intensive care units worldwide. With potent anti-inflammatory activities, glucocorticoid drugs were considered as an obvious choice for ALI treatment and had been tested in multiple clinical trials over the past several decades. However, these studies failed to show a clear clinical benefit of glucocorticoid drugs for ALI/ARDS treatment due to their immune-related side effects caused by nonspecific drug accumulation in healthy organs after systemic administration. A technology to deliver these drugs more specifically to the disease site and to confine or even increase their immunosuppressive activity in the disease site while reducing the side effects in healthy organs is highly desirable.

Here, we tackled this problem by covalently conjugating glucocorticoid drugs with a peptide that selectively targets the lung tissue under inflammatory conditions. Via an in vitro phage screen on a macrophage cell line, a cyclic peptide (CRVLRSGSC, termed CRV, from the first three residues) was identified, in which two terminal cysteines render the peptide cyclic by forming a disulfide bond. Using a murine lung infection model by bacteria, we found that CRV selectively homes to the infected lung and predominantly colocalizes with macrophages, but not healthy organs, upon intravenous injection. CRV was able to improve the delivery of porous silicon nanoparticles (pSiNPs) and achieve a higher efficacy of pSiNP-drug complex to attenuate infection-induced acute inflammation. We have identified retinoid X receptor b (RXRB) as the CRV receptor, whose cell-surface presence is limited to a subset of macrophages in the tumor tissue. It remains to be further clarified in regard to CRV affinity to other types of myeloid cells under various pathological conditions.

Macrophages and neutrophils account for the majority of the immune infiltrates in ALI. We speculated that RXRB expression may change with the inflammatory status in the lung and that CRV may facilitate the drug delivery to the inflamed lung. Although CRV-pSiNP formulation has shown promising effects, a significant accumulation of nanoparticles (NPs) was still found in liver and spleen due to the large sizes of NPs. Therefore, we directly conjugated CRV with prednisolone (PSL) in this study. To release the drug, a reactive oxygen species (ROS)-responsive linker was used between CRV and PSL. ROS is mainly found intracellularly and is excessively generated under inflammatory conditions. This design aimed to ensure that CRV-PSL is stable in the circulation while PSL is only cleaved from CRV after entering the cells in response to intracellular ROS. In this study, we explored the specificity of CRV to recognize the inflamed lung in a murine ALI model and tested whether our CRV-PSL conjugate exhibits a stronger therapeutic efficacy and safety profile than free PSL.

### Materials and Methods

### Chemicals and Reagents

Carboxyfluorescein-conjugated peptides<FAM>-<Ahx>-CRVLRSGSC (SEQ ID NO: 1; FAM-CRV) and <FAM>-<Ahx>-GGSGGSKG (SEQ ID NO: 2; FAM-GGS), <FAM>-Cys and <FAM>-Cys-<Ahx>-CRVLRSGSC (SEQ ID NO: 1; FAM-Cys-CRV) were purchased from LifeTein (Somerset, NJ). Maleimide-PEG2000-Hydroxyl (HO-PEG2000-MAL) was purchased from Nanosoft Polymers (Winston-Salem, NC). Prednisolone was purchased from Acros Organics (Carlsbad, CA). 2,2′-thiodiacetic acid, N, N′-dicyclohexylcarbodiimide (DCC), 4-dimethylaminopyridine (DMAP) and N, N-dimethylformamide (DMF) were purchased from Sigma-Aldrich (St Louis, MO). All other solvents and reagents used were of analytical quality. RPMI-1640 Medium were from Sigma-Aldrich. All reagents and compounds were used without further purification or modification.

### Synthesis of CRV-PSL Conjugates

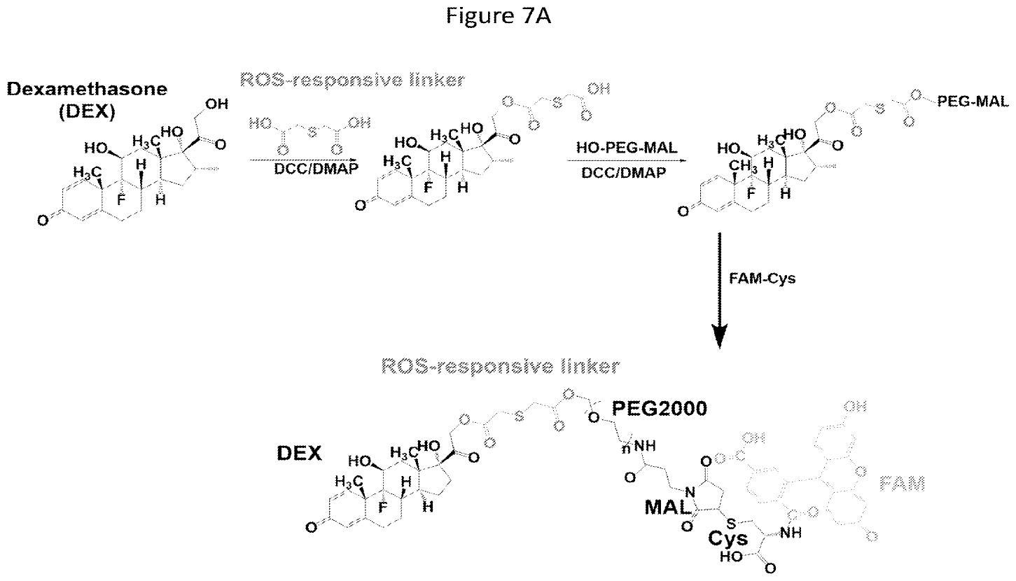

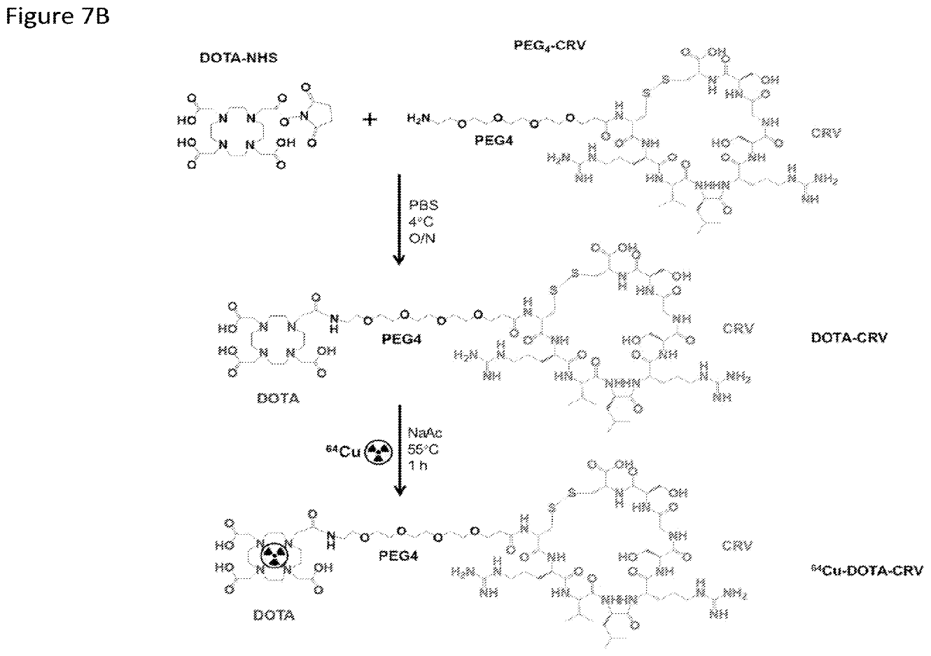

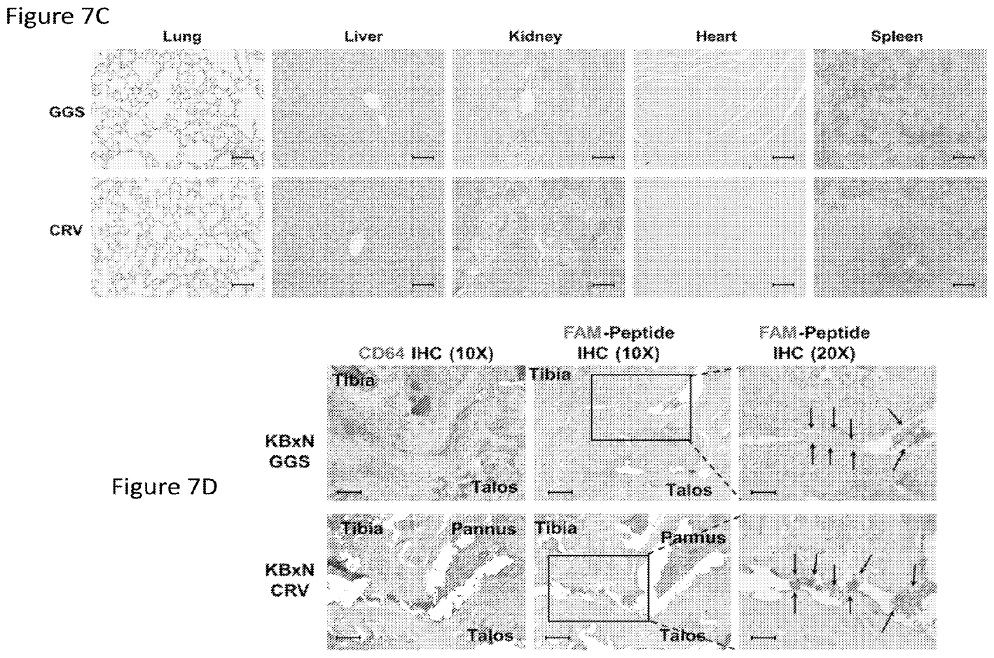

FAM-Cys-CRV-PSL (FAM-CRV-PSL) and FAM-Cys-PSL were designed and synthesized as shown in FIG. 7A-B. In brief, to construct an ROS-responsive drug delivery system, equimolar amounts of PSL and 2,2′-thiodiacetic acid were solved in DMF with magnetic stirring at 80° C., then 1.5 equivalents of DMAP and 3 equivalents of DCC in DMF were added to the solution. After overnight reaction, the DCC derived urea was removed using a cellulose/cotton filter. The product was purified by preparative HPLC (mobile phase: acetonitrile (ACN) & water, gradient elution: 10% ACN>90% ACN, column: Luna© 5 μm 250*10 mm C18). The dried product was mixed with an equimolar amount of HO-PEG2000-MAL and dissolved in DMF followed by 1.5 equivalents of DMAP and 3 equivalents of DCC in DMF with magnetic stirring at room temperature. After 2 hours, the DCC derived urea was removed using a cellulose/cotton filter. To precipitate the product, 30-40 times the volume of ethanol and diethyl ether were added and washed 3 times. The sedimentary product was dissolved in PBS with 10% DMF and was then mixed with 3 equivalents of FAM-Cys/FAM-Cys-CRV. The carboxyfluorescein dye FAM was attached through a cysteine linker to the N-terminus of CRV peptide for tracking. After 2 hours, the final reaction product (prodrug) was purified using a 2 kDa dialysis bag and was dried by lyophilization. The chemical structures of the prodrugs were verified by ¹H NMR.

### Cell Culture

RAW 264.7 cells were purchased from the American Type Culture Collection (ATCC, Manassas, VA). The cells were cultured in RPMI-1640 medium containing 50 U per mL streptomycin, 100 U per mL penicillin, and 10% fetal bovine serum (FBS, Thermo Fisher Scientific, Waltham, MA). The cells were cultured at 37° C. in a humidified incubator with 5% CO₂. All the cell cultures were maintained in 25 cm² cell culture flasks, 75 cm² cell culture flasks, or 10 cm culture dishes for use.

### Animals

All animal studies were carried out in compliance with the National Institutes of Health guidelines and an approved protocol from University of Minnesota (UMN) Animal Care and Use Committee. The animals were housed in a specific pathogen-free facility with free access to food and water at the Research Animal Resources (RAR) facility of the University of Minnesota.

C57BL/6Ncrl mice were purchased from the Charles River Laboratory (Wilmington, MA). The LPS-induced ALI model was established as previously described. Briefly, 8- to 9-week-old C57BL/6Ncrl male mice were given 25 μg of LPS (from Escherichia coli 0111:B4, Sigma) in 50 ul of PBS intratracheally. Sham-operated control animals underwent the same procedure with intratracheal injection of PBS. The mice were euthanized 4 days after LPS challenge for tissue collection and analysis.

### Human Interstitial Lung Disease Tissue

The study using lung tissue from human subjects with the interstitial lung disease was approved by the UMN IRB #STUDY00009145. The lung tissues of human subjects were obtained from the Clinical & Translational Science Institute in UMN. The tissues were collected from the patients that were clinically diagnosed with ILD, and hematoxylin-eosin (H&E) slides from each patient were sent to pathology in UMN to confirm the diagnosis. The controls were normal lungs obtained after confirmation via histology.

HPLC-MS/MS assay

HPLC-MS/MS analysis was performed using the chromatographic system consisted of UltiMate™ 3000 RSLCnano System (Thermo Fisher Scientific) and a ZORBAX™ C18 column (5 μm, 150 mm 0.5 mm, Agilent, Santa Clara, CA). The mobile phase was a mixture of H2O/acetonitrile with a gradient elution (from 90:10 to 10:90 and return to 90:10, v/v) at a flow rate of 15 μl/min. The eluent was introduced directly into the electrospray source of a tandem quadrupole mass spectrometer (TSQ VANTAGE™, Thermo Fisher Scientific) that was operated in the positive mode. The spray voltage was set at 3000 V. The compounds were analyzed by multiple reaction monitoring (MRM) of the transitions of m/z (ESI+) 361.24→147.06 for PSL.

### Intracellular Drug Release

Intracellular ROS-responsive drug release of PSL from CRV-PSL was investigated via HPLC-MS/MS assay. Confluent RAW cells were treated with 10 μM of free PSL or CRV-PSL, with or without 10 ng/ml of LPS, for 4 hours. After incubation, the cells were washed twice with cold PBS. The cells were then mixed with 500 μl methanol/dichloromethane (DCM) (1:1) via vortex for 1 min and centrifuged at 16000 g for 10 min. The upper liquid layer was collected, blown-dry with nitrogen, and dissolved in 20 μl of a mixture of acetonitrile (ACN): water (90:10, v/v). Then, 10 μl was injected for HPLC-MS/MS analysis. The PSL metabolites were analyzed by multiple reaction monitoring (MRM) of the transitions of mass to charge ratio (m/z) (ESI+) 363>345 for 20-α-OH-PSL and 20-β-OH-PSL.

### In Vivo Pharmacokinetics Assay

Healthy male C57BL/6 mice (8- to 9-weeks-old; body weight of 25-30 g) were deprived of food overnight with free access to water. The mice were randomly divided into three groups and were injected via lateral tail vein with free PSL, Cys-PSL, or CRV-PSL at a dose of PSL equivalent to 1 mg kg⁻¹ of free PSL. Approximately 100 μL of blood was collected into tubes via venous plexus at 5, 10, 30, 60 min and, 2, 6, 12, 24, and 48 h. The blood was centrifuged at 3000 rpm for 10 min to obtain plasma. Ten microliters of 0.1 M sodium hydroxide were added and vortexed for 30 seconds. Then, the plasma samples were mixed with 200 μl methanol/DCM (1:1) by vortex for 1 min and centrifuged at 16000 g for 10 min. The upper liquid layer was collected, blown-dry with nitrogen, and dissolved in 100 μl ACN for HPLC-MS/MS analysis.

### In Vivo Drug Distribution

After the establishment of acute lung injury by intratracheal injection of LPS, male C57BL/6 mice (8- to 9-weeks-old; body weight of 25-30 g) were deprived of food overnight with free access to water. The mice were randomly divided into three groups and were given free PSL, Cys-PSL, or CRV-PSL at a dose of PSL equivalent to 1 mg kg⁻¹ of free PSL via intravenous injection. After 2 hours, the mice were euthanized by transcardial perfusion with PBS and tissues (i.e., the heart, lung, liver, spleen, and kidney) were harvested and weighted. The tissues were then homogenized and dissolved in 200 μl of methanol/DCM (1:1). After a 1 min vortex followed by centrifugation at 16000 g for 10 min, the upper layer was removed and blown dry with nitrogen. The sample was then dissolved in 100 μl of ACN for HPLC-MS/MS assay.

### Real-Time RT-PCR

Total RNA was extracted from cells using TRI™ reagent (Sigma-Aldrich). First-strand cDNA was synthesized from total RNA using an iScript™ cDNA Synthesis Kit (Bio-Rad Laboratories, Hercules, CA). Quantitative amplification by PCR was performed using PowerUp SYBR Green qPCR Master Mix™ (Thermo Fisher Scientific) using a StepOne Real-Time PCR System™ (Applied Biosystem, Foster City, CA). The ΔΔCt method was used to determine the results. The housekeeping gene TATA-binding protein (TBP) was used as an endogenous control for quantification. The following primer pairs used in the Examples are listed in table 1.

TABLE 1
List of primer sequences used in this work.

| Primer | | Sequence | SEQ ID NO: |
|---|---|---|---|
| Human | | | |
| hRXRB | Forward | GCTGTGGAACAGAAGAGTGACC | SEQ ID NO: 3 |
| | Reverse | CCCACTCAACAAGCGTGAATAGC | SEQ ID NO: 4 |
| hIL1β | Forward | ATGATGGCTTATTACAGTGGCAA | SEQ ID NO: 5 |
| | Reverse | GTCGGAGATTCGTAGCTGGA | SEQ ID NO: 6 |
| hTNFα | Forward | CCTCTCTCTAATCAGCCCTCTG | SEQ ID NO: 7 |
| | Reverse | GAGGACCTGGGAGTAGATGAG | SEQ ID NO: 8 |
| hIL6 | Forward | ACTCACCTCTTCAGAACGAATTG | SEQ ID NO: 9 |
| | Reverse | CCATCTTTGGAAGGTTCAGGTTG | SEQ ID NO: 10 |
| hMCP1 | Forward | GATCTCAGTGCAGAGGCTCG | SEQ ID NO: 11 |
| | Reverse | TTTGCTTGTCCAGGTGGTCC | SEQ ID NO: 12 |
| hMMP2 | Forward | AGCGAGTGGATGCCGCCTTTAA | SEQ ID NO: 13 |
| | Reverse | CATTCCAGGCATCTGCGATGAG | SEQ ID NO: 14 |
| hMMP3 | Forward | CACTCACAGACCTGACTCGGTT | SEQ ID NO: 15 |
| | Reverse | AAGCAGGATCACAGTTGGCTGG | SEQ ID NO: 16 |
| hMMP9 | Forward | GCCACTACTGTGCCTTTGAGTC | SEQ ID NO: 17 |
| | Reverse | CCCTCAGAGAATCGCCAGTACT | SEQ ID NO: 18 |
| hMMP13 | Forward | CCTTGATGCCATTACCAGTCTCC | SEQ ID NO: 19 |
| | Reverse | AAACAGCTCCGCATCAACCTGC | SEQ ID NO: 20 |
| hTIMP1 | Forward | GGAGAGTGTCTGCGGATACTTC | SEQ ID NO: 21 |
| | Reverse | GCAGGTAGTGATGTGCAAGAGTC | SEQ ID NO: 22 |
| hTIMP2 | Forward | ACCCTCTGTGACTTCATCGTGC | SEQ ID NO: 23 |
| | Reverse | GGAGATGTAGCACGGGATCATG | SEQ ID NO: 24 |
| hRPL37A | Forward | ATTGAAATCAGCCAGCACGC | SEQ ID NO: 25 |
| | Reverse | AGGAACCACAGTGCCAGATCC | SEQ ID NO: 26 |
| Mouse | | | |
| mIL1β | Forward | CCTTCCAGGATGAGGACATGA | SEQ ID NO: 27 |
| | Reverse | TGAGTCACAGAGGATGGGCTC | SEQ ID NO: 28 |
| mTNFα | Forward | GAGAAAGTCAACCTCCTCTCTG | SEQ ID NO: 29 |
| | Reverse | GAAGACTCCTCCCAGGTATATG | SEQ ID NO: 30 |
| mIL6 | Forward | CCAGAAACCGCTATGAAGTTCCT | SEQ ID NO: 31 |
| | Reverse | CACCAGCATCAGTCCCAAGA | SEQ ID NO: 32 |
| miNOS | Forward | GAGACAGGGAAGTCTGAAGCAC | SEQ ID NO: 33 |
| | Reverse | CCAGCAGTAGTTGCTCCTCTTC | SEQ ID NO: 34 |
| mIL18 | Forward | GACAGCCTGTGTTCGAGGATATG | SEQ ID NO: 35 |
| | Reverse | TGTTCTTACAGGAGAGGGTAGAC | SEQ ID NO: 36 |
| mCcl2 | Forward | GTTGGCTCAGCCAGATGCA | SEQ ID NO: 37 |
| (Mcp1) | Reverse | AGCCTACTCATTGGGATCATCTTG | SEQ ID NO: 38 |
| mMMP12 | Forward | CACACTTCCCAGGAATCAAGCC | SEQ ID NO: 39 |
| | Reverse | TTTGGTGACACGACGGAACAGG | SEQ ID NO: 40 |
| mTIMP1 | Forward | TCTTGGTTCCCTGGCGTACTCT | SEQ ID NO: 41 |
| | Reverse | GTGAGTGTCACTCTCCAGTTTGC | SEQ ID NO: 42 |
| mTBP | Forward | GAAGAACAATCCAGACTAGCAGCA | SEQ ID NO: 43 |
| | Reverse | CCTTATAGGGAACTTCACATCACAG | SEQ ID NO: 44 |
| mTGF-b1 | Forward | AAACGGAAGCGCATCGAA | SEQ ID NO: 45 |
| | Reverse | GGGACTGGCGAGCCTTAGTT | SEQ ID NO: 46 |
| mColI-a1 | Forward | CAACCTGGACGCCATCAAG | SEQ ID NO: 47 |
| | Reverse | CAGACGGCTGAGTAGGGAACA | SEQ ID NO: 48 |

### Flow Cytometry

To identify subpopulations of myeloid cells, single cell suspensions were collected from the lung with a lung dissociation kit for mouse (Miltenyi Biotec, Auburn, CA) and other tissues, including blood and spleen, were collected for flow cytometry analysis. Single-cell suspensions with 0.5-1×10⁶ cells from tissue digests were stained with fluorochrome-conjugated antibodies as indicated in the results section. The antibody panel is shown in Table 2. Flow cytometry analysis was performed on a BD Fortessa™ X-20 (BD Biosciences, East Rutherford, NJ). The data were analyzed with Flow JO™ software (Tree Star, San Carlos, CA).

TABLE 2
List of antibodies used in this work

| Antibody | Supplier | Catalog No. | Clone |
|---|---|---|---|
| TruStain fcX ™ (rat anti-mouse | Biolegend | 101320 | 93 |
| CD16/32) antibody | | | |
| Rabbit polyclonal anti-RXRB | GeneTex | GTX79265 | — |
| antibody | | | |
| Rabbit anti-fluorescein/Oregon | Invitrogen | A889 | — |
| Green | | | |
| Rat anti-mouse CD11b | Abcam | AB8878 | M1/70 |
| Rat anti-mouse F4/80 | Abcam | AB6640 | A3-1 |
| Rat anti-Mouse CD64 | Fisher | PIMA523993 | 290322 |
| Donkey anti-rabbit IgG-AF 488 | Invitrogen | A21206 | — |
| Donkey anti-rabbit IgG-AF 594 | Invitrogen | A21207 | — |
| Donkey anti-rat IgG-AF 594 | Invitrogen | A21209 | — |
| Rat anti-mouse/human | Biolegend | 101236 | M1/70 |
| CD11b-Brilliant violet 421 | | | |
| Rat anti-mouse | Biolegend | 123110 | RM8 |
| F4/80-PE | | | |
| Rat Anti-Mouse | BD | 561037 | 30-F11 |
| CD45-APC-Cy7 | Bioscience | | |
| Rat anti-mouse | Biolegend | 100235 | 17A2 |
| CD3-APC | | | |
| Rat-anti-mouse | Biolegend | 115507 | 6D5 |
| CD19-PE | | | |
| Rat anti-mouse | Biolegend | 127627 | 1A8 |
| Ly-6G-Brilliant Violet 421 | | | |
| Rat anti-mouse | BD | 563005 | 1A8 |
| Ly-6G-Brilliant Violet 605 | Bioscience | | |

### Hematoxylin-Eosin (H&E) Staining and Lung Injury Score

The lung tissues were harvested from ALI mice after 3 days of treatment with free PSL, Cys-PSL and CRV-PSL, respectively. The formalin-fixed tissues underwent tissue processing and then were embedded in paraffin to create a formalin-fixed, paraffin embedded block for histology. Hematoxylin and eosin (H&E) staining was performed on paraffin-embedded sections to assess the morphologic changes in injured lung tissue. After deparaffinization and rehydration, 4-μm sections of the lung tissues were stained with H&E according to standard procedures. To evaluate the lung injury score, each slide was examined by an independent investigator in a blinded manner. A total of 300 alveoli were counted on each slide at 40× magnification as described previously. The injury score was calculated according to the following formula: injury score=[(alveolar hemorrhage points/no. of fields)+2×(alveolar infiltrate points/no. of fields)+3×(fibrin points/no. of fields)+(alveolar septal congestion/no. of fields)]/total number of alveoli counted×100.

### Western Blotting

Fifty micrograms of protein from each cell lysate were subjected to electrophoresis in SDS-PAGE on 4%-15% precast protein gels (Bio-Rad, Hercules, CA) and then transferred to polyvinylidene fluoride (0.2 mm pore size) membranes using a miniblot apparatus (Bio-Rad). The membranes were probed with primary antibodies including mouse antibodies to IL-1b and tubulin (Cell Signaling Technology, Danvers, MA); and rabbit polyclonal antibody to iNOS (Thermo Fisher Scientific) and monoclonal antibody to glyceraldehyde-3-phosphate dehydrogenase (GAPDH; Cell Signaling) as appropriate with constant shaking overnight at 4° C. After washing, the bound antibodies were detected with the secondary antibodies IR-Dye 680RD donkey anti-rabbit immunoglobulin G (IgG) and IRDye™ 800CW donkey anti-mouse IgG (Li-COR, Lincoln, NE). The mem-branes were scanned via the Odyssey CLx™ imaging system (LI-COR), and the images with blots were quantified by ImageJ software.

### Immunohistochemistry (IHC) Staining

Immunohistochemical staining was performed on paraffin-embedded sections. In brief, after deparaffinization and rehydration, all sections were incubated with 0.3% H2O2 solution to block endogenous peroxidase activity. The sections were then blocked with 5% donkey serum blocking solution (with 0.1% triton X100), followed by incubation with primary antibodies overnight at 4° C. The primary antibodies included polyclonal rabbit anti-RXRB (GeneTex, Irvine, CA), anti-fluorescein/Oregon Green (Invitrogen, Waltham, MA), anti-iNOS (PA1-036), anti-myeloperoxidase antibody (PA5-16672, Thermo Fisher Scientific), rat anti-CD11b, and CD64 (Thermo Fisher Scientific). After treatment with secondary anti-rabbit (HRP) antibody for 1 h and then DAB peroxidase (HRP) substrate (Vector Labs, Burlingame, CA) for 1 min, hematoxylin counterstaining was performed followed by dehydration in ethanol and xylene. The stained samples were mounted with Permount™ (Thermo Fisher Scientific). Images of randomly selected areas from each section were collected using the microscope. Five views per tissue were captured from each section for semi-quantification via ImageJ™ software according to the HRP-DAB signal.

### Immunofluorescence (IF) Staining

Immunofluorescence staining was performed on the paraffin-embedded or frozen tissue sections. In brief, the sections were first treated with PBS containing 1% BSA and 0.1% Triton X100 (blocking buffer) at room temperature (RT) for 1 h. The sections were then washed three times with PBS and were incubated with primary antibodies at a 1:200 dilution in blocking buffer at 4° C. overnight, followed by the appropriate secondary antibodies diluted 1:200 in blocking buffer at RT for 1 h. The primary antibodies used were as follows: rabbit anti-fluorescein/Oregon Green (Invitrogen), anti-RXRB (GeneTex), rat anti-CD11b, CD64, and F4/80 (Thermo Fisher Scientific). After washing with PBS, sections were stained with Hoechst or DAPI, mounted in mounting medium, and covered with a coverslip. The sections were examined using the fluorescence microscope EVOS™ M5000 (Thermo Fisher Scientific).

### TUNEL Staining

TUNEL staining was performed using an In Situ Cell Death Detection Kit (Roche Diagnostics GmbH, Mannheim, Germany). The paraffin sections were stained with TUNEL reaction mixture for 1 h at 37° C. followed by Hoechst staining for nucleus. The images were captured by using fluorescence microscopy (EVOS™ M5000) and the fluorescence intensity of TUNEL positive cells was evaluated using ImageJ software for semi-quantification.

### Biochemical Toxicity Analysis

Aminotransferase quantification was performed to determine the liver toxicity of different formulations of PSL. Blood samples were collected by cardiac puncture after the treatment. The levels of aspartate aminotransferase (AST) and alanine aminotransferase (ALT) in plasma were measured using assay kits from Stanbio (Boerne, TX).

### Statistical Analysis

A test of normal distribution of the variants was performed followed by one-way analysis of variance (ANOVA) or Student's t-test, as appropriate, to assess differences among groups. The qPCR results were expressed as means±standard error of mean (SEM). Other results were expressed as mean±standard deviation (SD). A p-value less than 0.05 was considered significant.

### Results:

### CRV Selectively Targets the Myeloid Cells in the Inflamed Lung Due to Elevated RXRB Expression

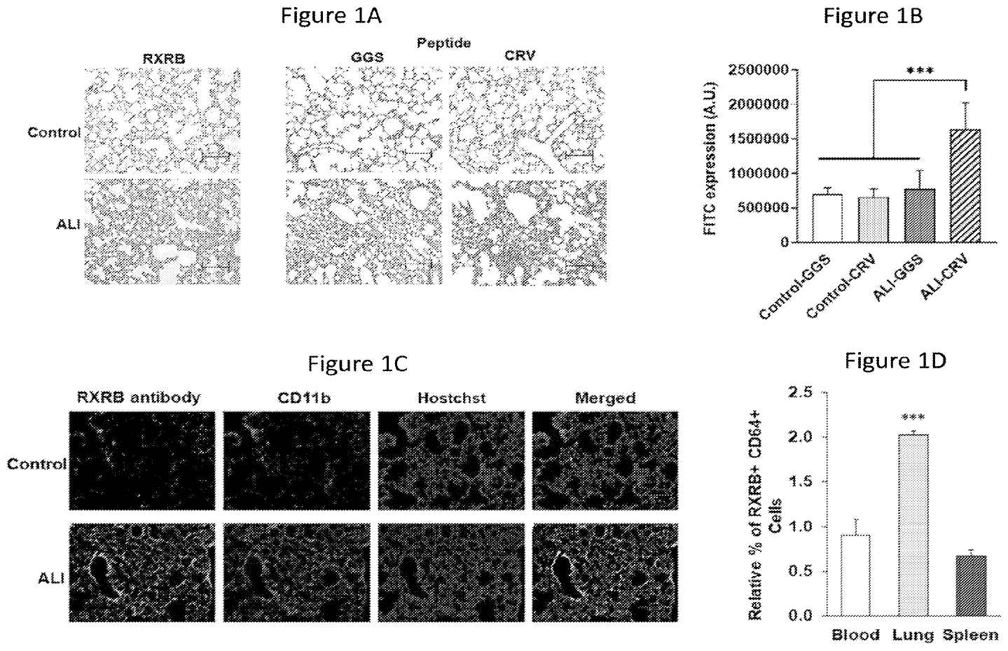

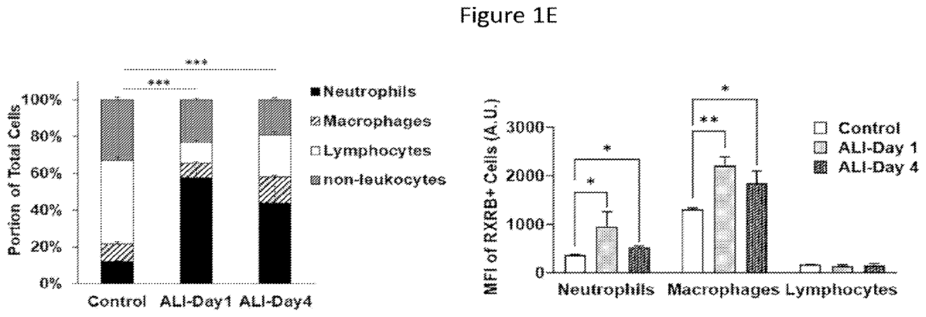

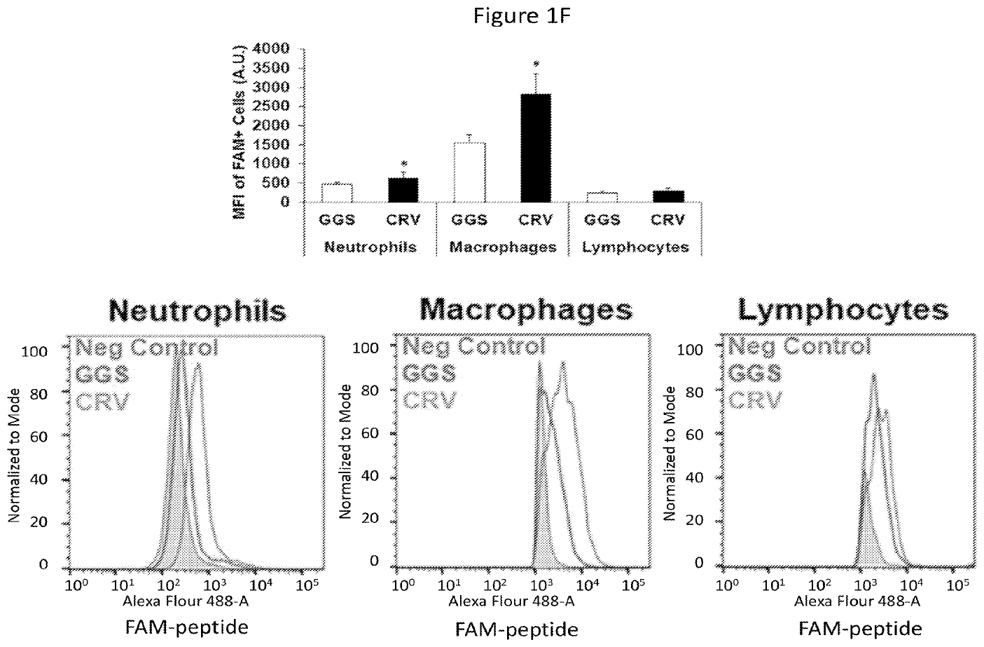

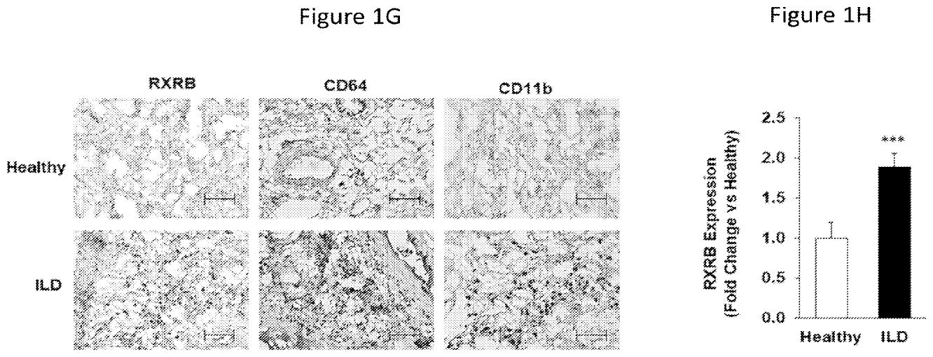

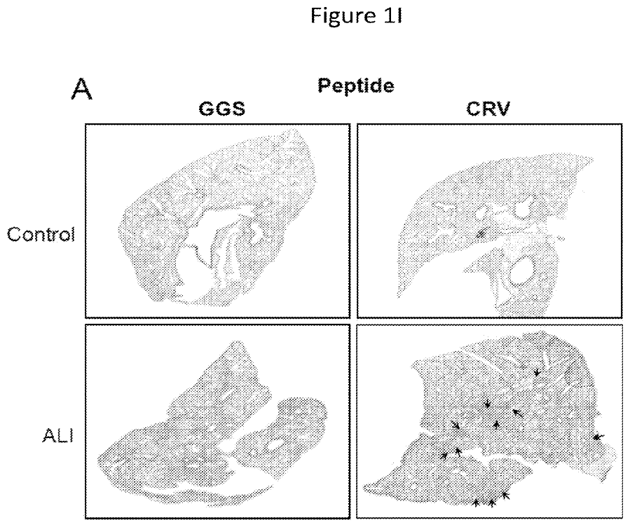

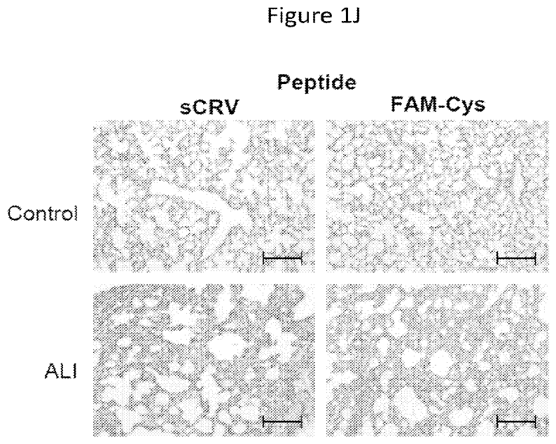

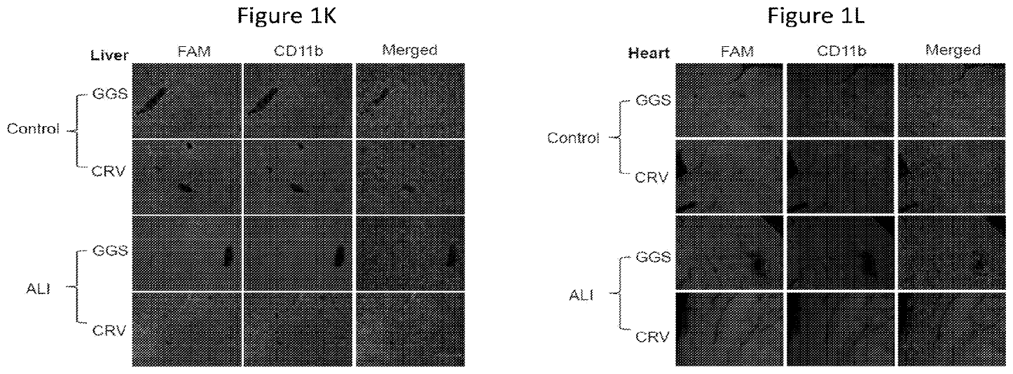

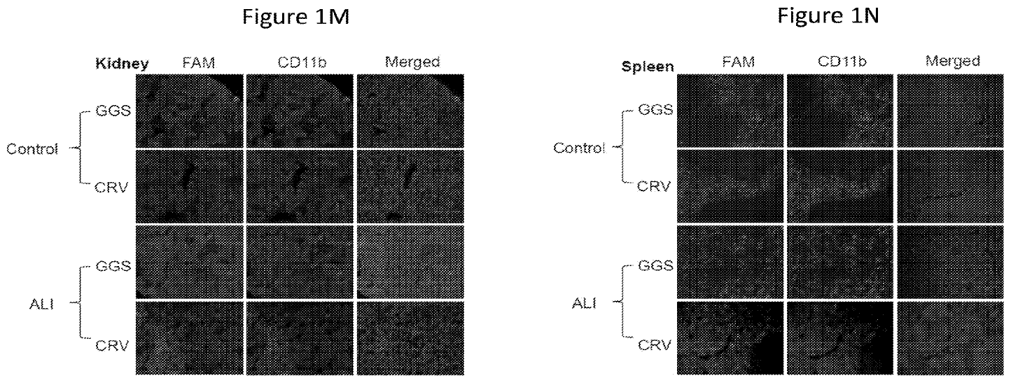

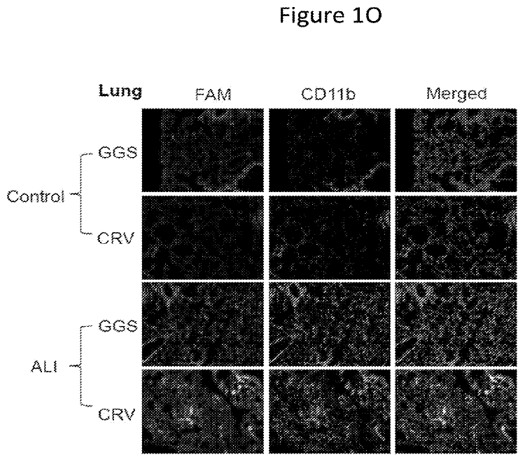

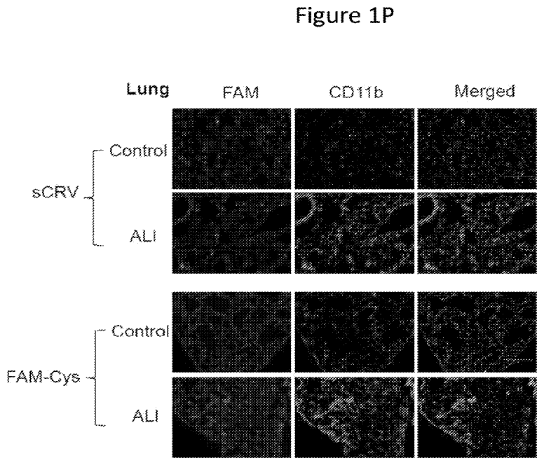

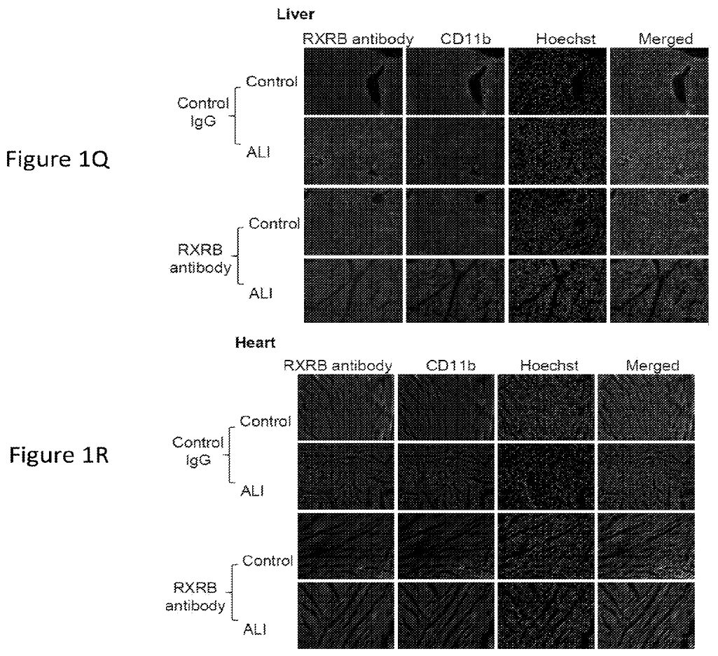

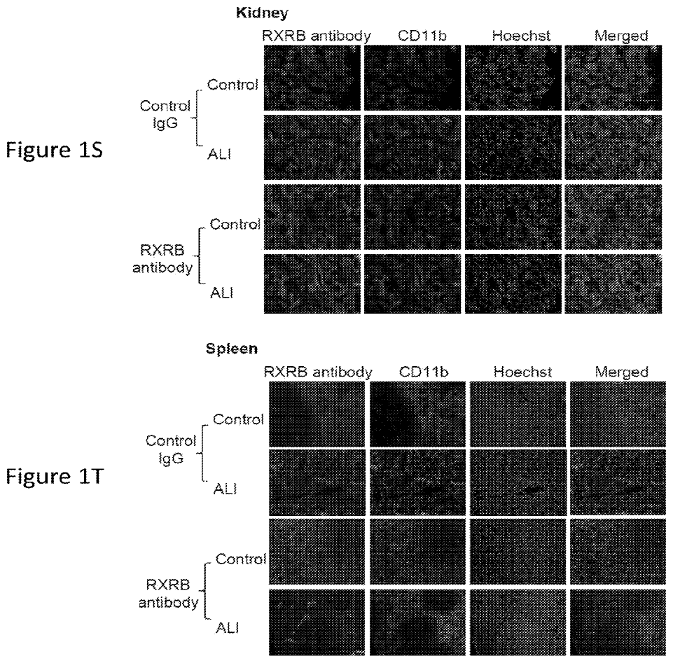

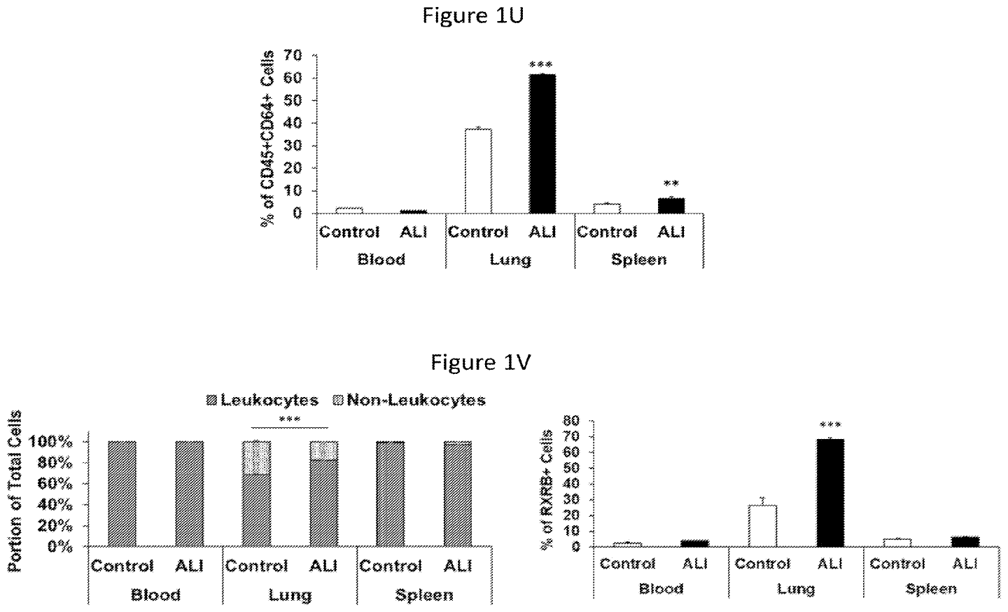

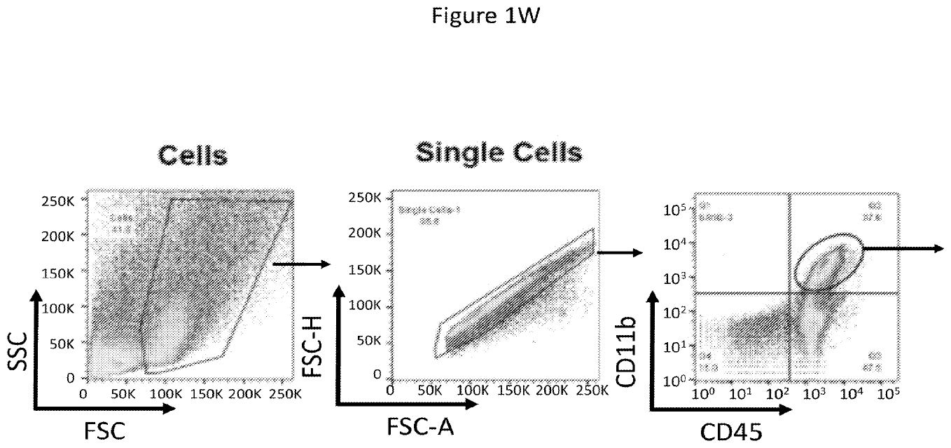

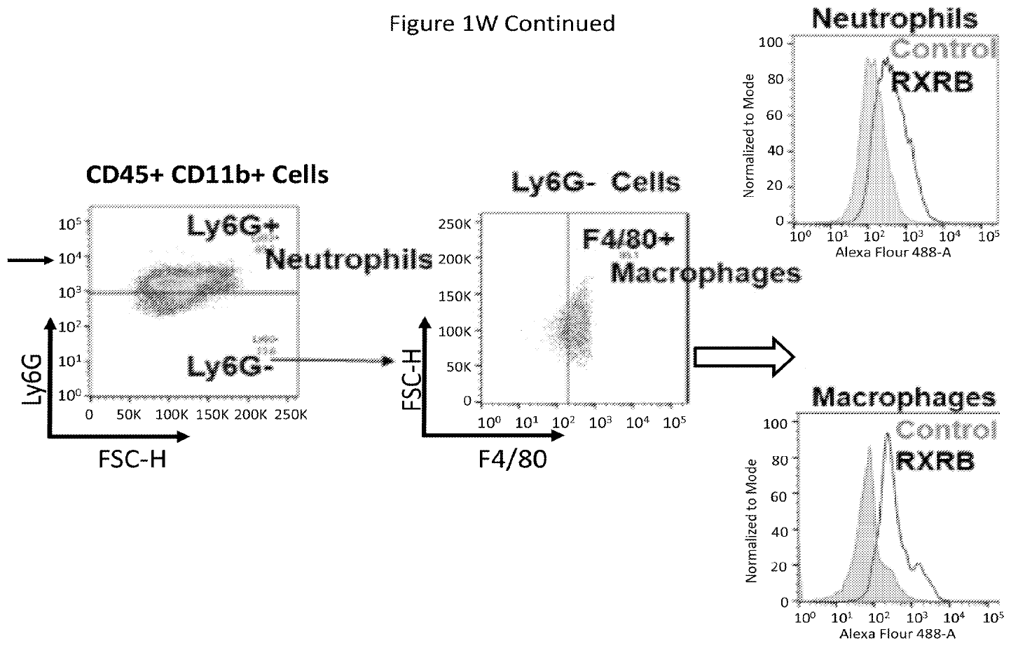

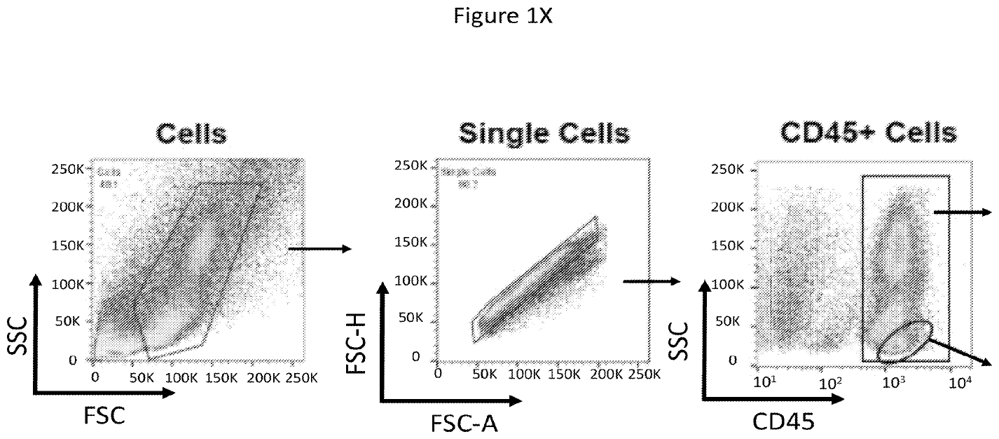

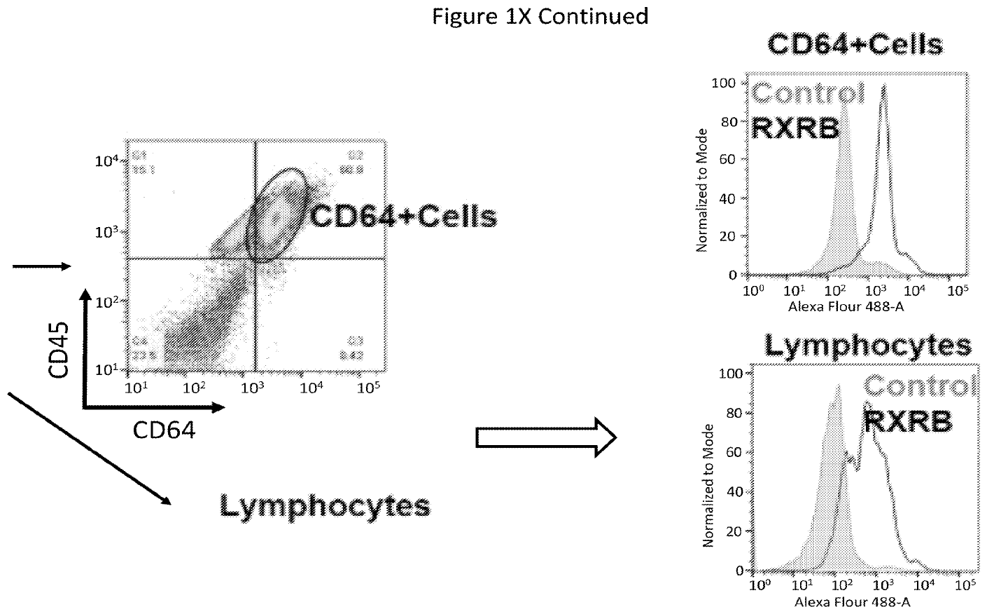

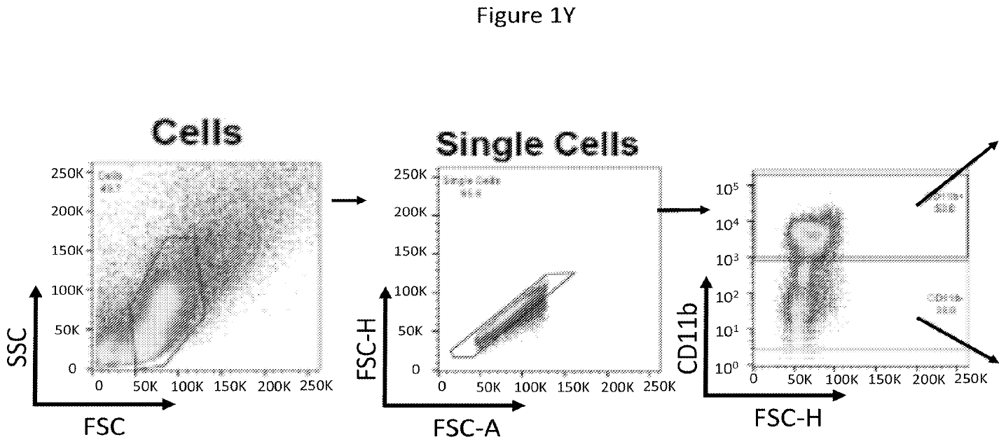

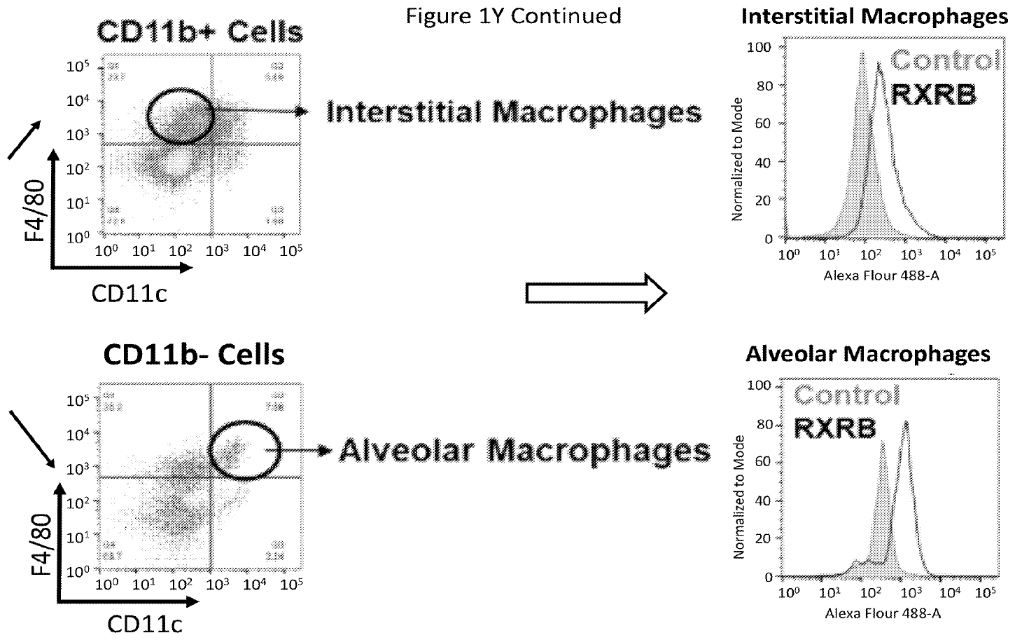

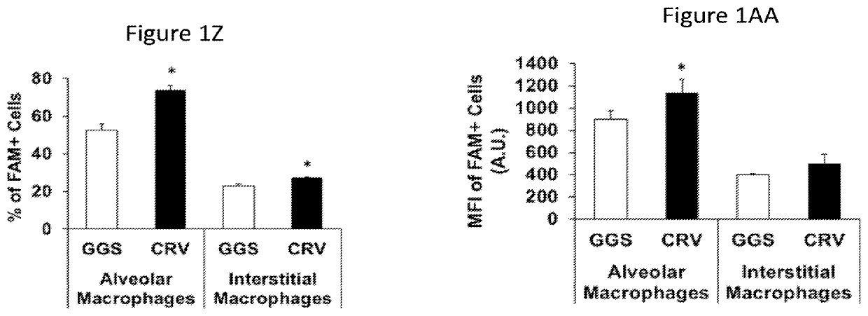

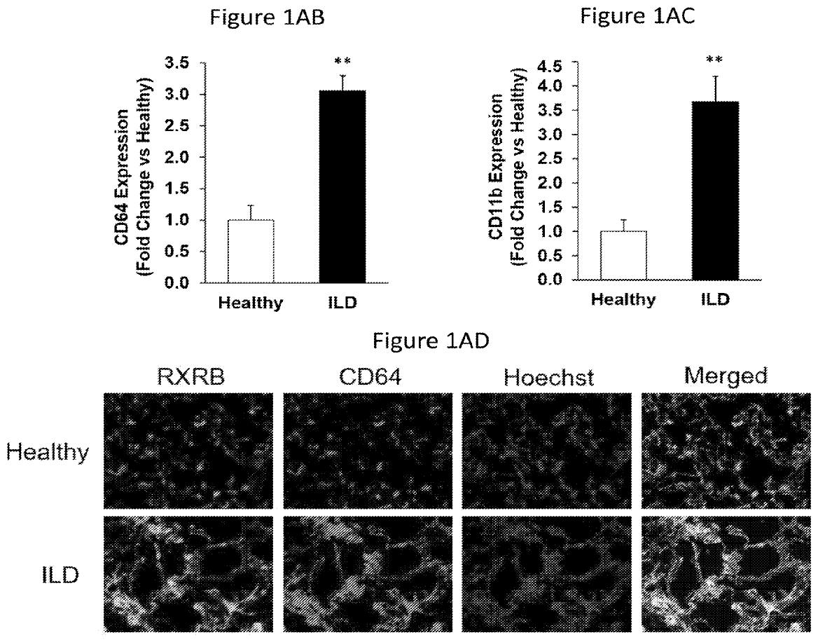

In this study, we primarily used an endotoxin-induced ALI model by intratracheal injection of lipopolysaccharide (LPS), which is one of commonly used murine models to mimic the inflammatory responses of ALI.13 We first tested the overall RXRB expression in this ALI model, and CRV homing upon systemic administration. RXRB expression increased in the lung tissue upon LPS stimulation (FIG. 1A). At 4 days after ALI induction, fluorescein amidite (FAM)-CRV was injected intravenously into the mice for 1-h treatment before the lung and control organs were collected. Immunohistochemistry (IHC) and immunofluorescence (IF) studies showed that CRV accumulates in the inflamed lung at a significantly higher level than control peptides, including GGS, scrambled CRV, and FAM-Cys (CRV replaced with a single cysteine), but there was little accumulation of these peptides in the healthy lung (FIGS. 1A, 1B, 1I, and 1J). In healthy organs, these peptides exhibited little accumulation with or without LPS stimulation (FIG. 1I, 1J). CRV predominantly colocalized with CD11b-positive immune cells in the inflamed lung but not in healthy tissues (FIGS. 1I-1P). When given intravenously, an anti-RXRB antibody also showed lung-specific accumulation in ALI mice but not in healthy mice or healthy organs of ALI mice (FIGS. 1C and 1Q-1T).

To further understand the basis of CRV targeting, we investigated the cell-surface RXRB expression in the immune cells of healthy and ALI lungs, as surface RXRB determines the CRV binding to cells. Single cells of healthy and ALI lungs were first sorted with CD45 for leukocytes, followed by CD64 to gate out myeloid infiltrates, mainly macrophages and neutrophils. The percentages of these inflammatory cells were significantly increased in the ALI lungs compared with healthy ones (FIG. 1U). Like our previous findings, a very small fraction of white blood cells or splenic cells of healthy or ALI mice was expressing RXRB on the surface, while the percentage of RXRB-positive leukocytes was significantly increased in the ALI lung compared with the healthy one (FIG. 1V). Accordingly, RXRB-positive portion of myeloid in-filtrates (CD64-positive) also significantly increased in the ALI lung compared with healthy ones, but not for their counterparts in blood or spleen (FIG. 1D). It was already known that the neutrophil recruitment occurs after 3-6 h of LPS treatment and reaches peak on day 3-4 after stimulation together with macrophage infiltration. To have a kinetic view, we investigated the immune subsets (neutrophils [Ly6G+], macrophages [Ly6G-F4/80+], and lymphocytes) in the lung after 24 h and 4 days of LPS injection. Agreeing with previous reports, the fraction of myeloid infiltrates significantly increased in the lung at either onset or 4 days after ALI (FIG. 1E). The surface expression of RXRB-positive cells in neutrophils and macrophages was significantly elevated in the ALI lung, but not for lymphocytes (FIGS. 1E, 1W, and 1X). We further isolated the cells from ALI lung to discriminate the interstitial macrophages (TM; CD11b+F4/80+CD11C−) and alveolar macrophages (AM; CD11b-F4/80+CD11C+). Both types of macrophages have elevated RXRB expression, suggesting the possibility of CRV targeting (FIG. 1Y). To corroborate the above results, we detected the content of CRV in different immune subsets of ALI lungs after intravenous injection. Macrophages showed the highest CRV signal per cell, indicating a preferential binding or internalization ability (FIG. 1F). CRV exhibited a specific binding (higher than GGS) to neutrophils and macrophages, but not lymphocytes (FIG. 1F). Upon CRV injection, CRV binds more AM than IM (FIGS. 1Z and 1AA). Overall, these results suggest that CRV mainly targets the myeloid infiltrates in the ALI lung likely due to the elevated level of cell-surface RXRB.

Besides the murine model, we also acquired a lung tissue specimen from patients with interstitial lung disease (ILD). Compared with subjects with healthy lungs, the level of RXRB was much higher in the lungs of ILD patients, as was the overall amount of myeloid infiltrates (FIGS. 1G, 1H, 1AB, and 1AC). We also found that RXRB colocalized well with CD64-positive cells (FIG. 1AD).

### Synthesis and In Vitro Characterization of CRV-Drug Conjugate

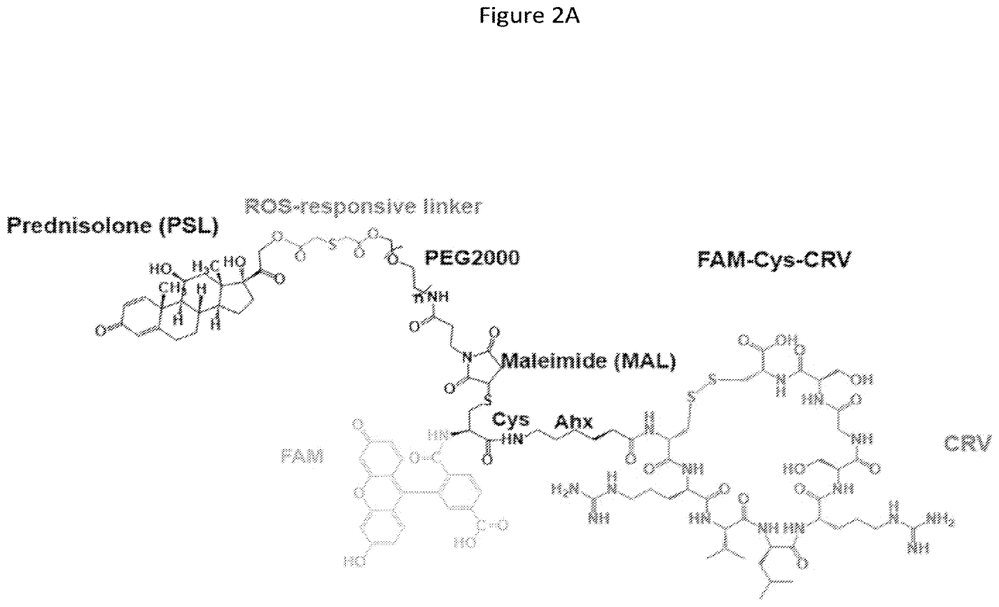

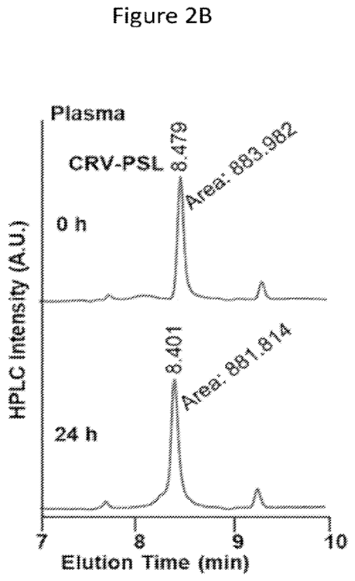

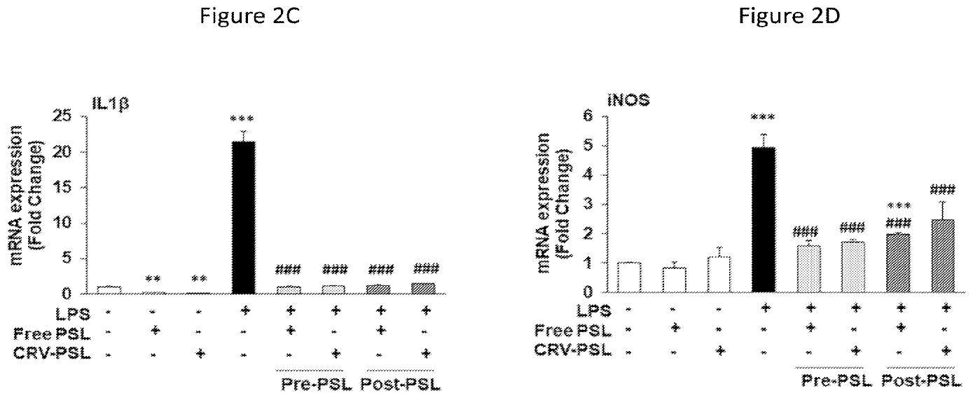

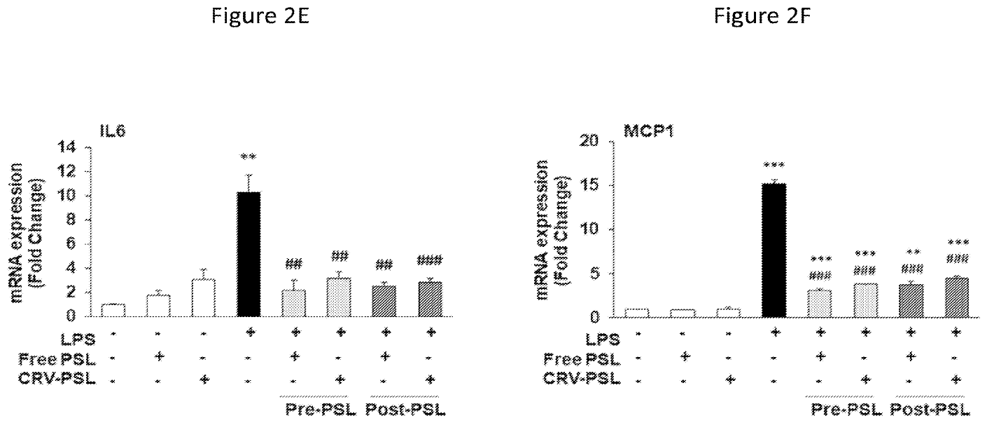

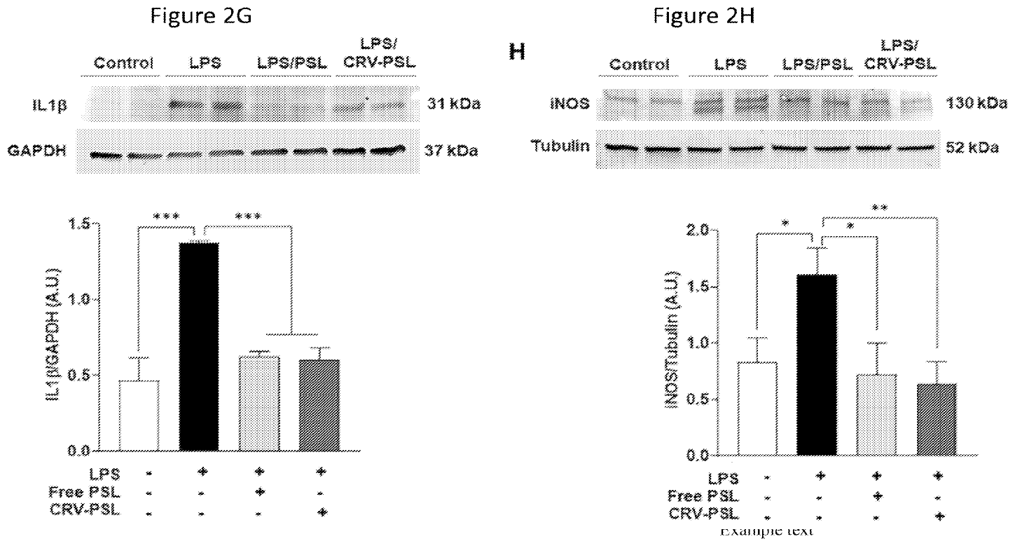

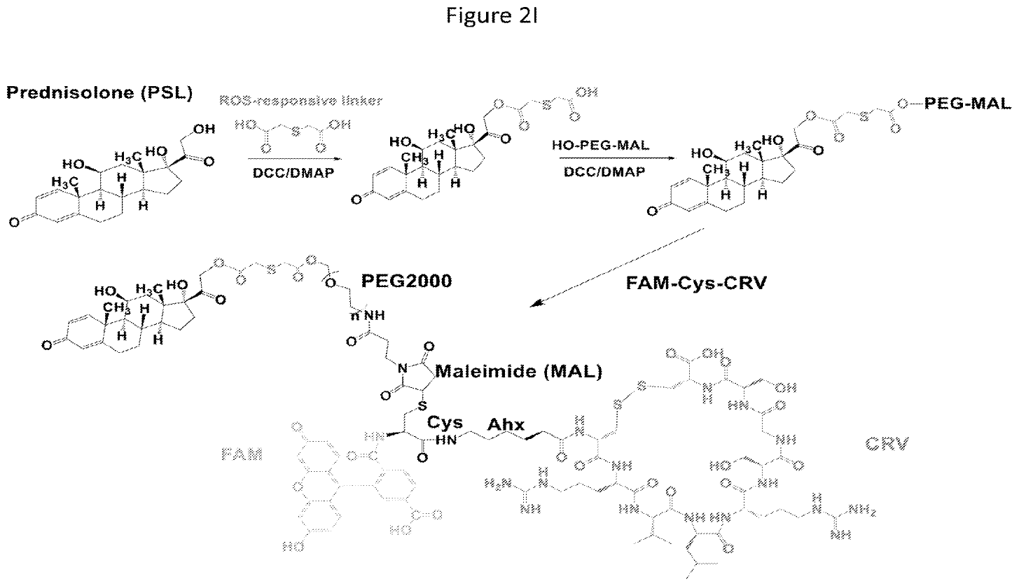

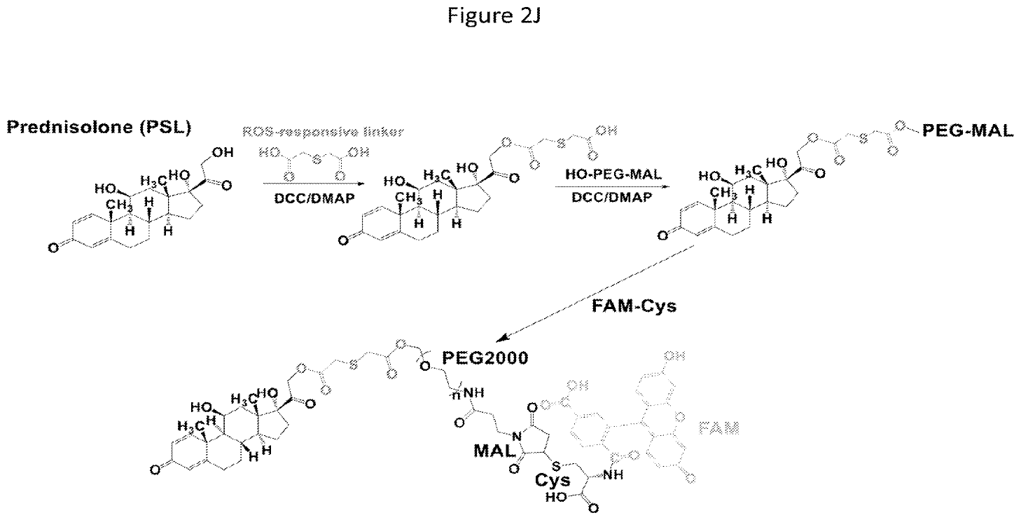

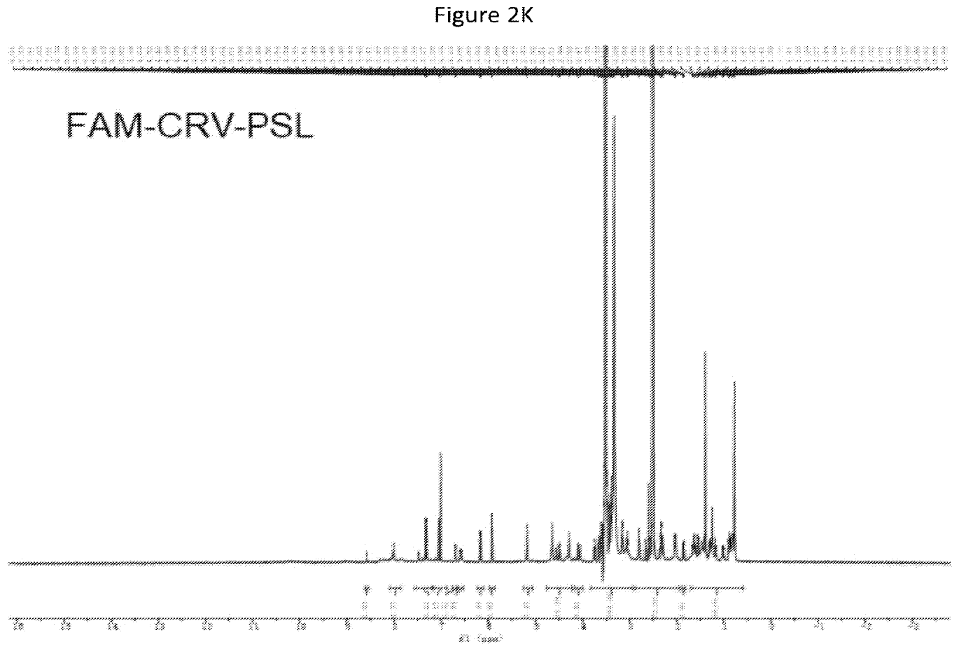

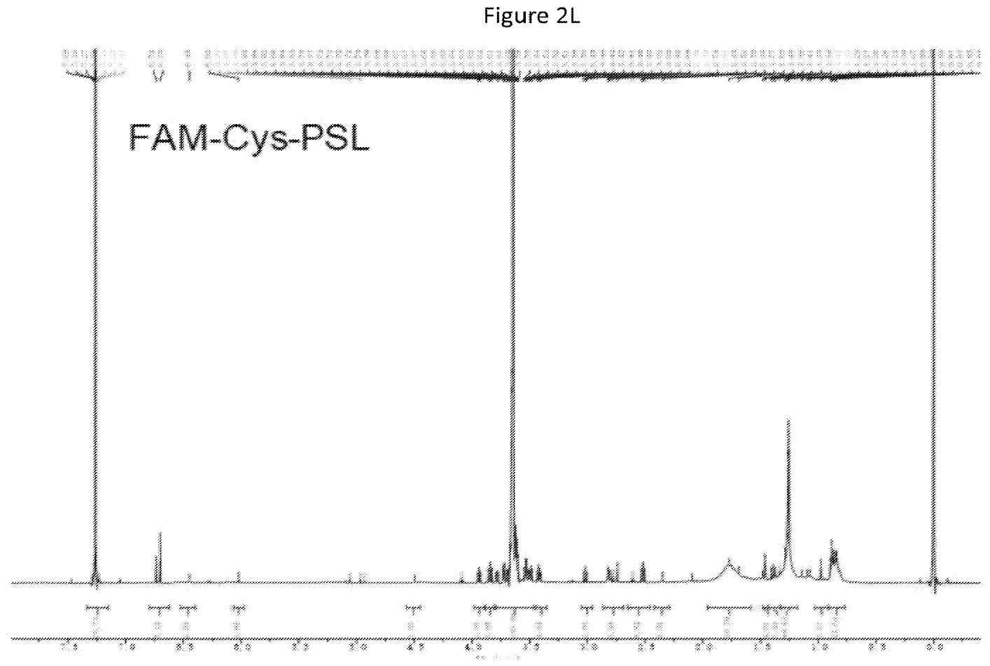

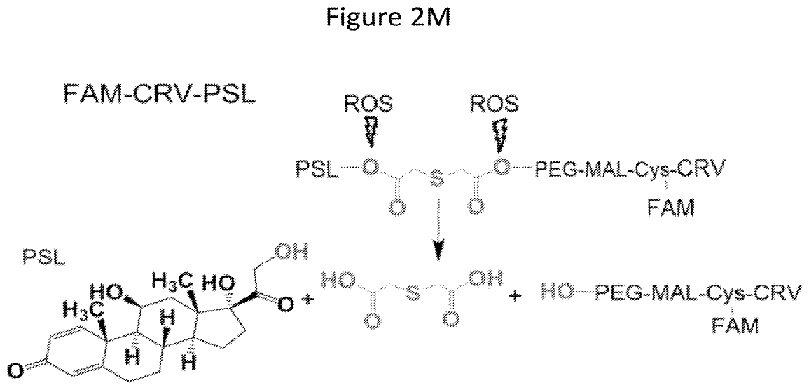

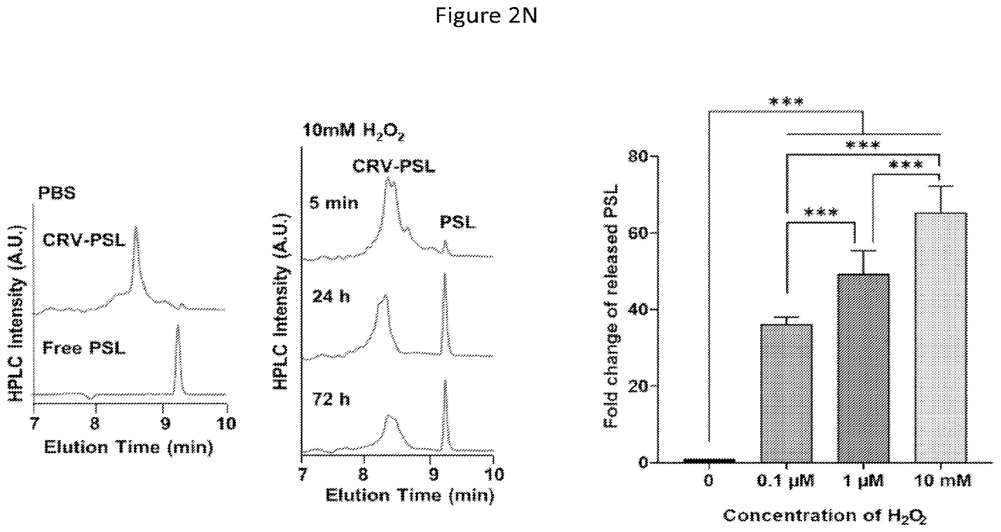

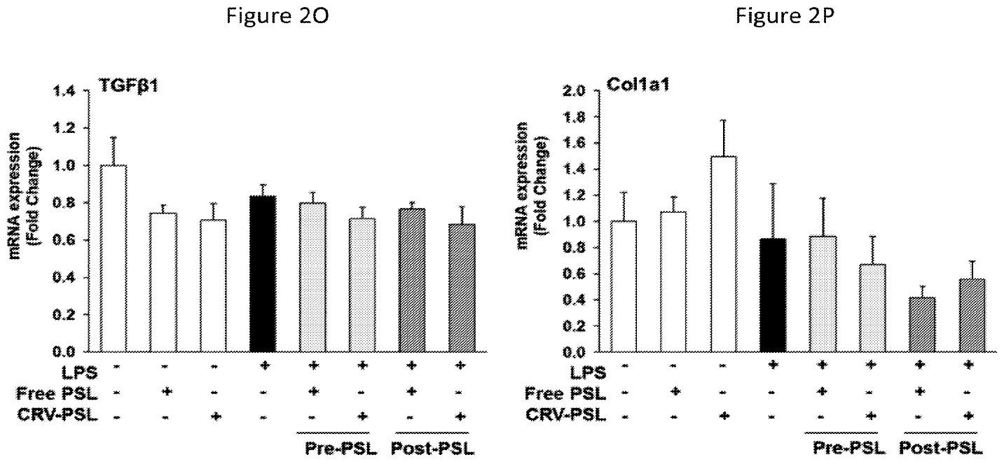

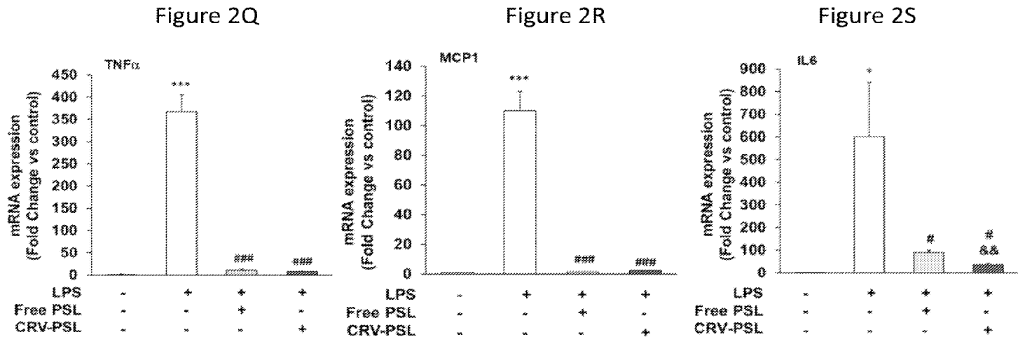

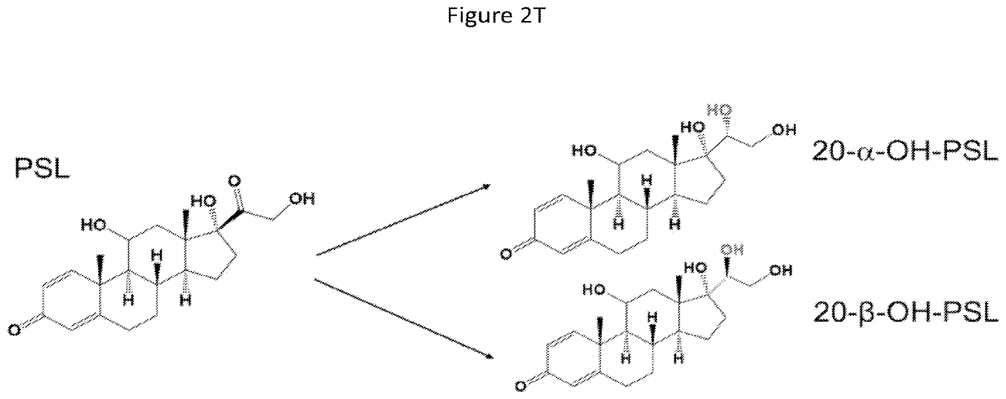

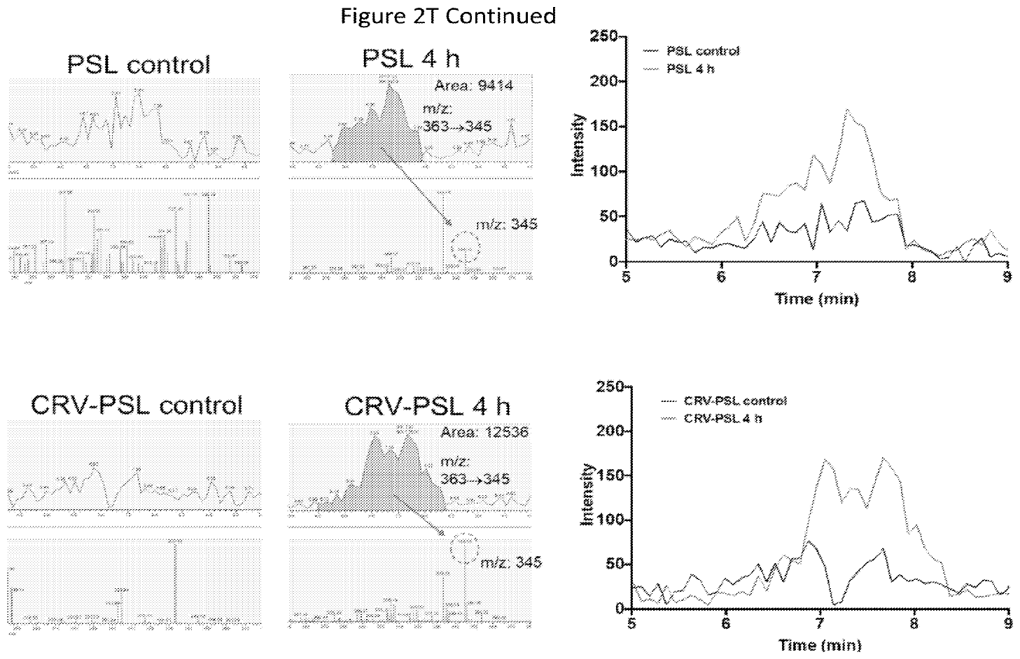

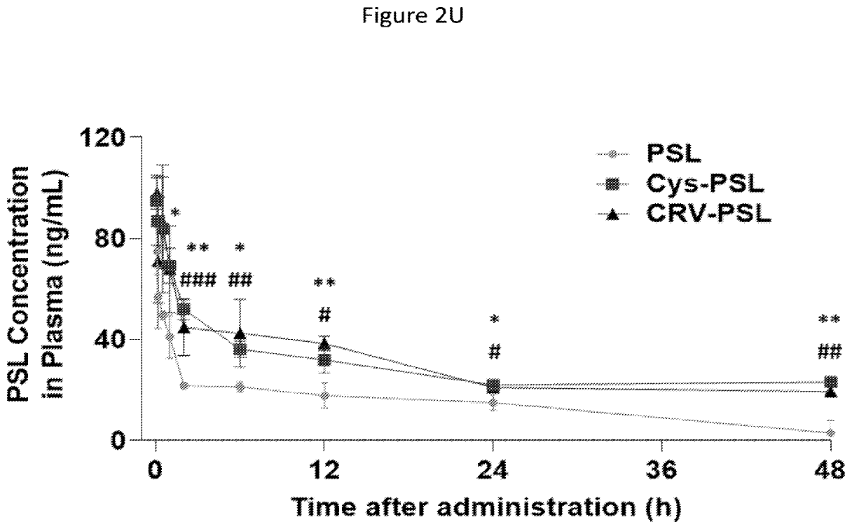

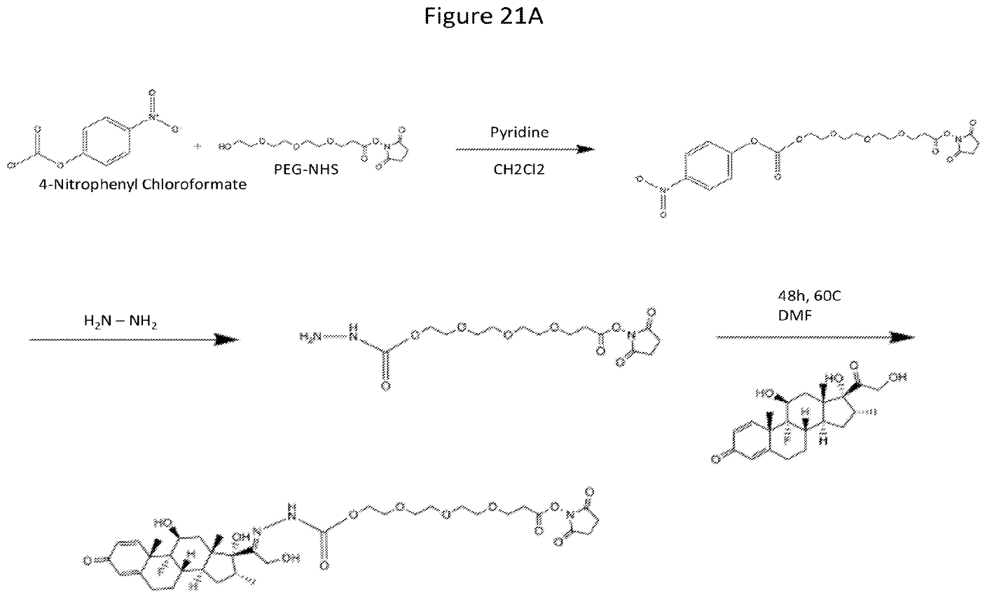

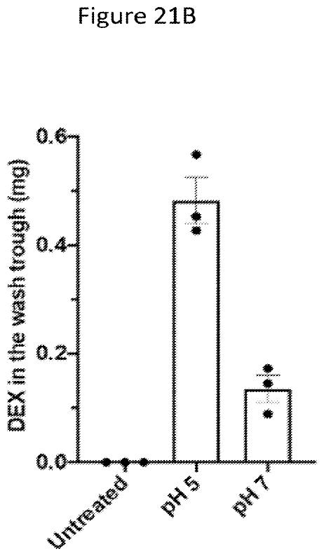

Next, we investigated whether CRV can improve the delivery of glucocorticoid drugs to the inflamed lung. PSL was used as a model glucocorticoid drug to covalently conjugate with CRV as schemed in FIG. 2A (hereafter CRV-PSL). The experimental conditions of conjugation reactions are described in more detail in materials and methods and shown in FIG. 21. We also replaced CRV with a single cysteine, termed Cys-PSL, as a control conjugate without targeting peptide (FIG. 2J). An ROS-responsive linker, 2,20-thiodiacetic acid, was used between PSL and CRV (FIG. 2A). The chemical structures of both PSL conjugates were verified by 1H nuclear magnetic resonance (NMR) (FIGS. 2K and 2L).

The ROS-responsive linker is expected to be cleaved under oxidative conditions in ALI, at which time PSL will be freed from the conjugated peptide (FIG. 2M). To validate this point, CRV-PSL was incubated with 10 mM H2O2 at 37° C. for 5 min, 24 h, and 72 h, respectively. The amount of CRV-PSL and PSL was determined by high-performance liquid chromatography (HPLC). The cleavage of CRV-PSL into PSL was seen after 5-min incubation with H2O2 and became more evident after longer times of incubation (FIG. 2N). Moreover, H2O2 induced the release of free PSL from ROS-responsive conjugates in a dose-dependent manner (FIG. 2N). The majority of CRV-PSL remained uncleaved in the mouse plasma at least for 24 h (FIG. 2B). Overall, these results suggested that the ROS-responsive conjugation is stable in the circulation and allows the release of free PSL under oxidative conditions.

It was previously reported that polyethylene glycol (PEG) conjugation renders drugs, including corticosteroids, inactive until cleaved, after which drugs can become active and subject to metabolism. Therefore, our first and most important task of in vitro validation was to verify whether CRV-PSL has immunosuppressive activity equivalent to that of PSL. LPS-induced inflammatory responses have been shown to generate ROS in different cell types including RAW 264.7 macrophage cell line. Thus, as a proof-of-principle demonstration, we used RAW 264.7 cells to validate the intracellular cleavage and anti-inflammatory activity of CRV-PSL. CRV-PSL or PSL was added to the cells 1 h before LPS stimulation to exclude the influence of LPS on glucocorticoid receptor expression and functions (pre-PSL). We also added the drugs 1 h after LPS stimulation to mimic the drug treatment after the onset of inflammation in a clinical situation (post-PSL). At 18 h after drug treatment, LPS-induced inflammatory responses were measured based on the expression of proinflammatory markers including interleukin-1b (IL-1b), IL-6, monocyte chemoattractant protein-1 (MCP-1), and inducible nitric oxide synthase (iNOS); and anti-inflammatory/resolution markers such as transforming growth factor b1 (TGF-b1) and collagen type I a1 (ColI-a1). Under both pre- and post-PSL conditions, CRV-PSL exhibited similar activity as PSL to suppress LPS-induced inflammatory responses (FIGS. 2C-2F, 2O, and 2P). Consistently, CRV-PSL exhibited an inhibitory effect similar to that of PSL on LPS-induced inflammation as evaluated by the expression of IL-1b and iNOS at the protein levels (FIGS. 2G and 2H). Similar results were also found in human THP-1 macrophages (FIGS. 2Q-2S). Additionally, we also detected PSL metabolites inside cells, which is another indicator of PSL release. After cellular uptake, we detected several PSL metabolites, such as 20-α-OH-PSL and 20-b-OH-PSL, using mass spectrometry as previously reported. CRV-PSL was able to generate 20-α-OH-PSL and 20-b-OH-PSL in a level similar to that of free PSL (FIG. 2T). Together, the above results suggest that CRV-PSL can be processed to release free PSL in cells under oxidative stress and, more importantly, possess immunosuppressive activity similar to that of PSL.

### Pharmacokinetics and Biodistribution of CRV-PSL Conjugate In Vivo

We first evaluated the plasma pharmacokinetics of CRV-PSL in healthy mice. PSL, Cys-PSL, and CRV-PSL were injected intravenously at a dose of 1 mg kg⁻¹ (equivalent PSL weight), and their plasma half-life (T_{1/2}) and area under the plasma concentration curve (AUC) were calculated using a noncompartmental model (Table 3 and FIG. 2U). CRV and Cys conjugates showed a higher plasma concentration than PSL alone as early as 1 h after injection and remained until at least 48 h (FIG. 2U). Thus, both PSL conjugates showed a prolonged T_{1/2} and about 1-fold increase of AUC compared with PSL alone (Table 3).

TABLE 3
Pharmacokinetic parameters of PSL after intravenous
administration of free PSL, Cys-PSL, and CRV-PSL to
mice (1 mg kg−1 equivalent dose of PSL per mouse)

| Pharmacokinetic | | | |
|---|---|---|---|
| Parameters | Free PSL | Cys-PSL | CRV-PSL |
| T_{1/2}(h) | 3.78 ± 0.50 | 4.47 ± 0.22 | 6.13 ± 2.59 |
| AUC₀₋ₜ | 41,279.66 ± | 82,414.35 ± | 82,880.57 ± |
| | 5,527.07 | 3,511.63* | 3,611.63* |

Values are expressed as mean ± SD (n = 3),
*p < 0.001 relative to free PSL.
T½, half-life; AUC0-t, area under the concentration-time curve.

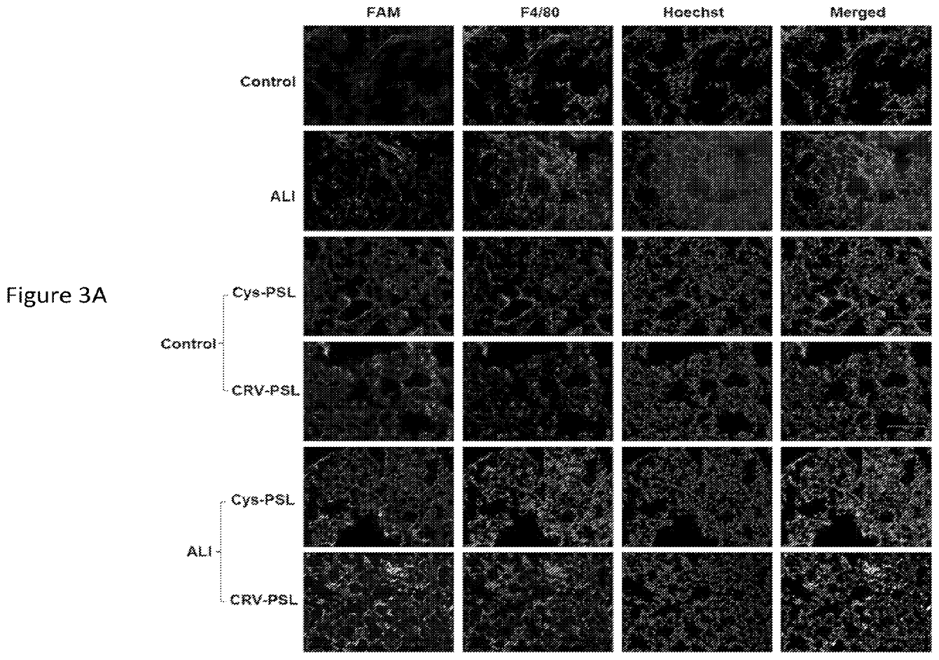

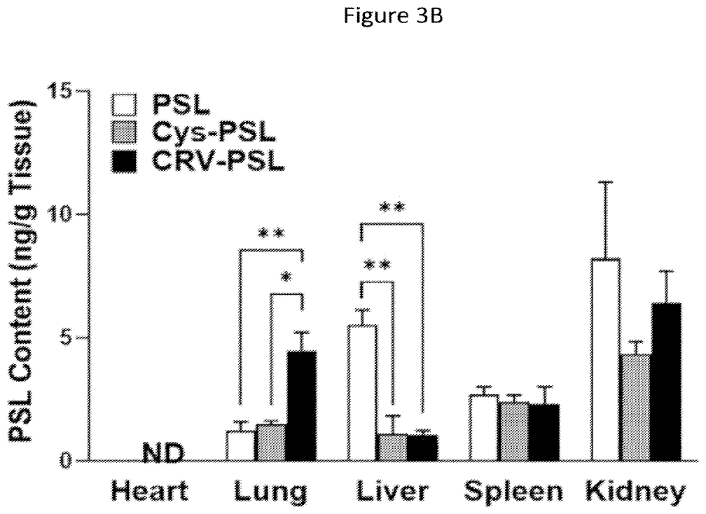

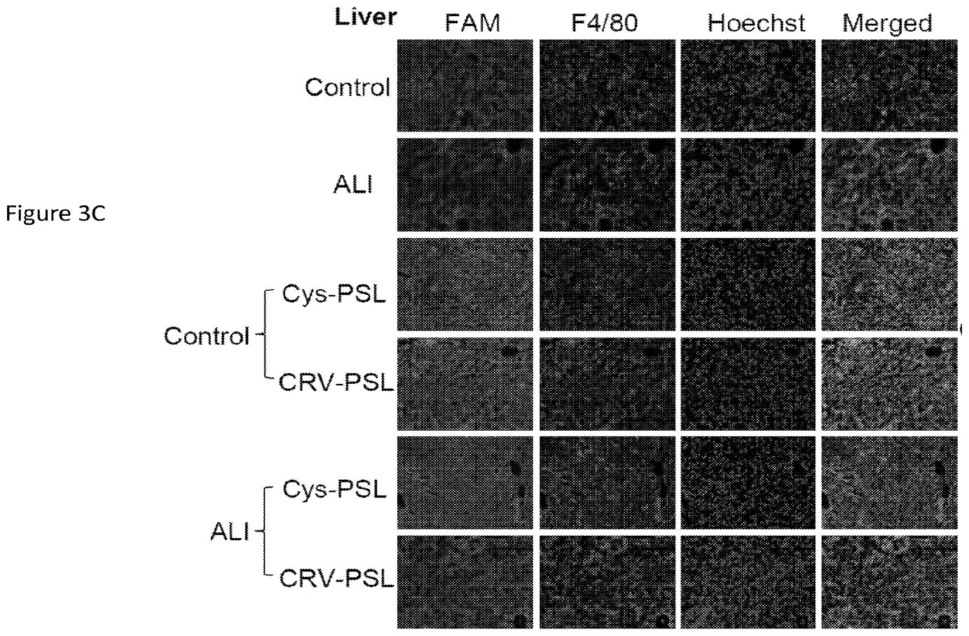

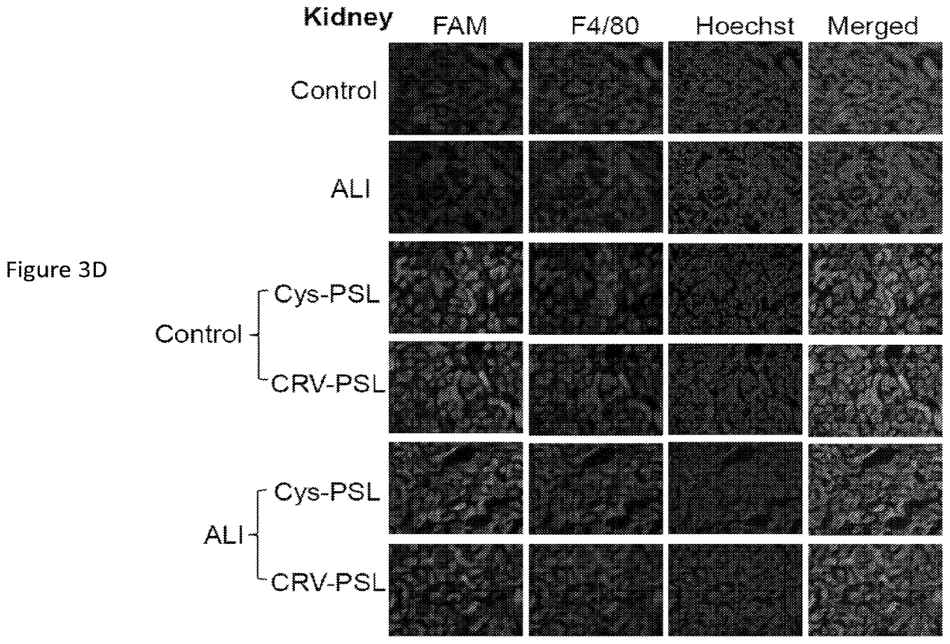

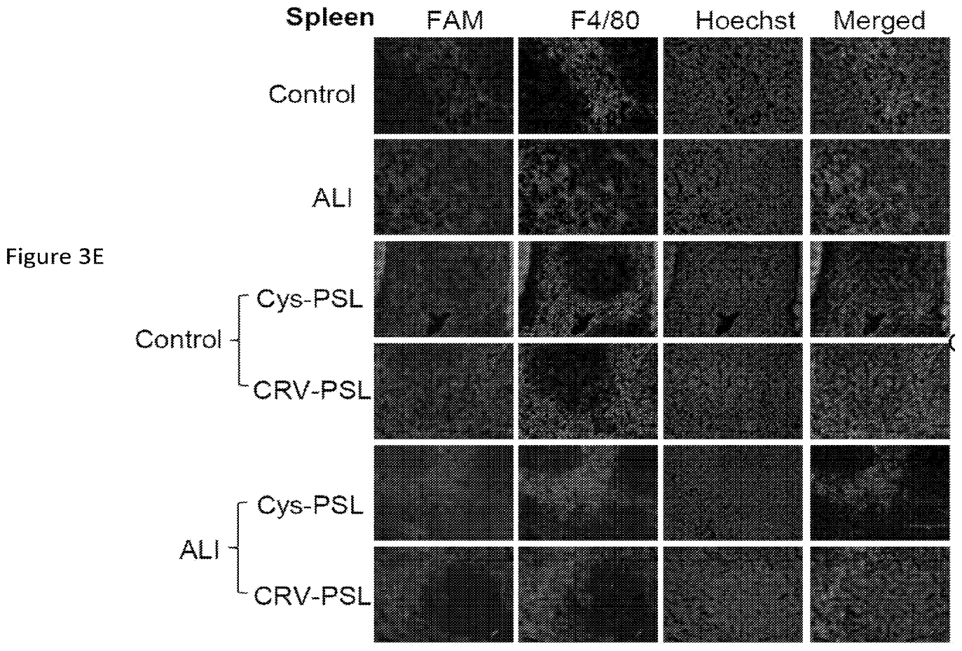

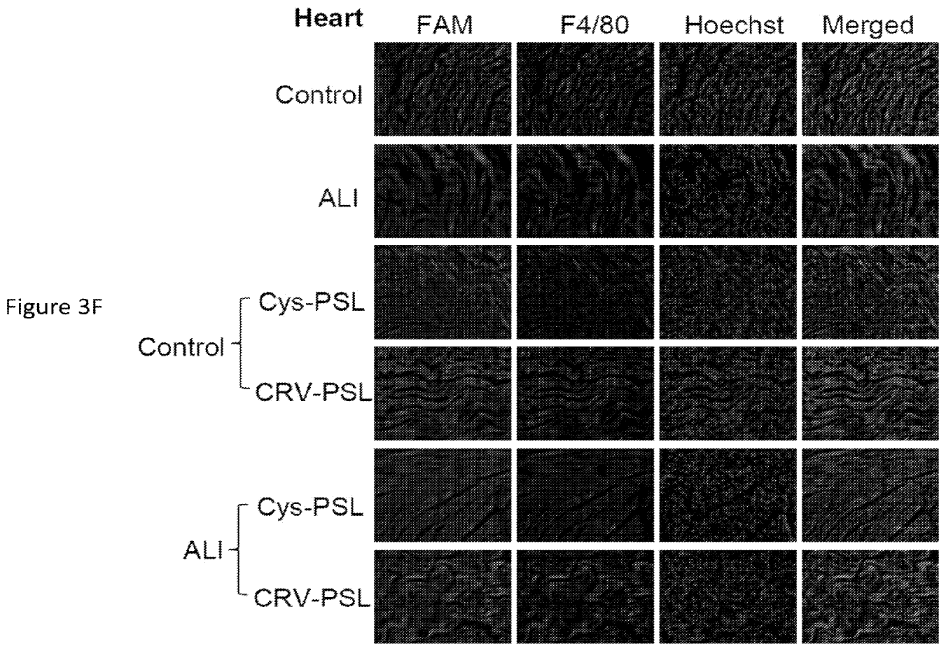

Next, we investigated the in vivo biodistribution of PSL conjugates in ALI mice. At 1 h after intravenous injection, IF staining revealed that CRV-PSL accumulation in the lung of ALI mice was significantly higher than that of Cys-PSL, while there was little accumulation of both conjugates in the lung of healthy mice (FIG. 3A). In the ALI lung, CRV-PSL colocalized well with F4/80-positive macro-phages (FIG. 3A). The accumulation of CRV-PSL was similar to that of Cys-PSL in healthy organs of both healthy and ALI mice (FIG. 3C-3F).

In the above studies, we detected and quantified the conjugates as one entity. However, it is free PSL that is the active component in inducing therapeutic efficacy and side effects. Therefore, we used mass spectrometry to directly quantify the amount of PSL in various tissues of ALI mice. ALI mice were intravenously injected with PSL, Cys-PSL, and CRV-PSL at a dose of 1 mg kg⁻¹ in PSL weight, respectively, and the tissues were collected at 2 h after injection. The CRV-PSL group exhibited a higher amount of PSL in the lung than PSL or Cys-PSL groups (FIG. 3B). In the meantime, the liver accumulation of PSL in Cys-PSL and CRV-PSL groups was lower than that of the PSL group (FIG. 3B). There was little difference among the three groups in other organs (no detectable PSL signal in the heart) (FIG. 3B). Collectively, these results demonstrated that CRV conjugation increases the PSL accumulation in the ALI lung, whereas it reduces it or has no impact in healthy organs.

### CRV Conjugation Increases the Therapeutic Efficacy of PSL Against Lung Injury

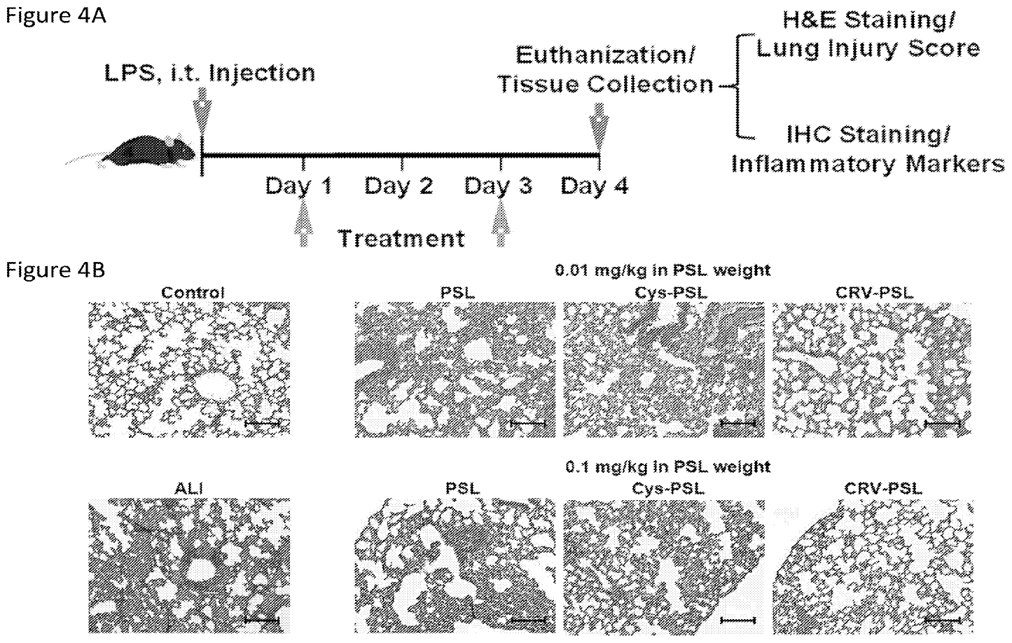

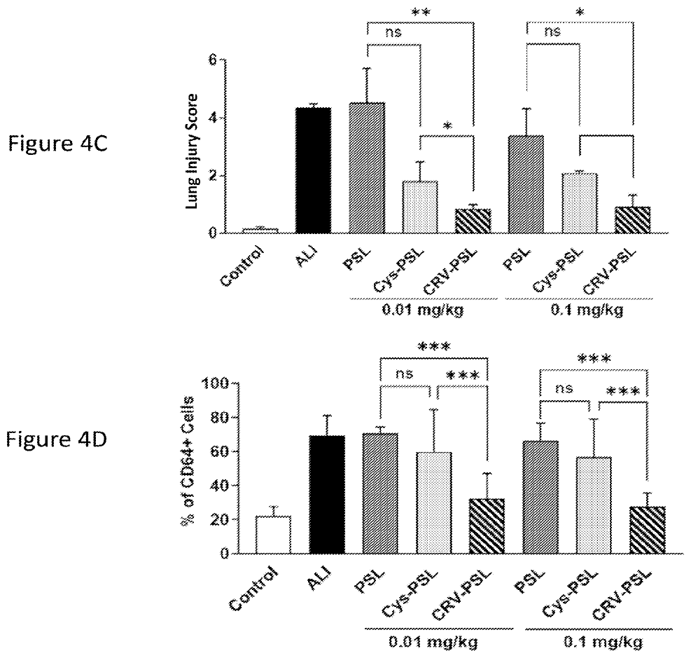

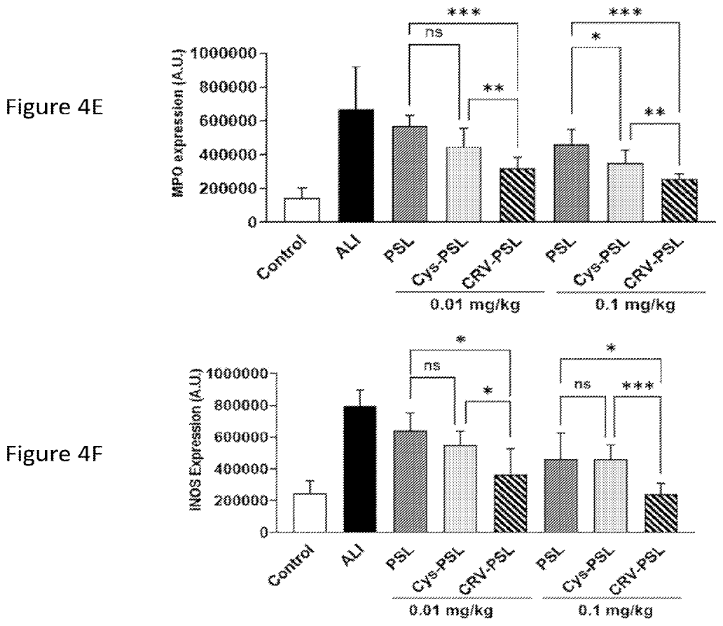

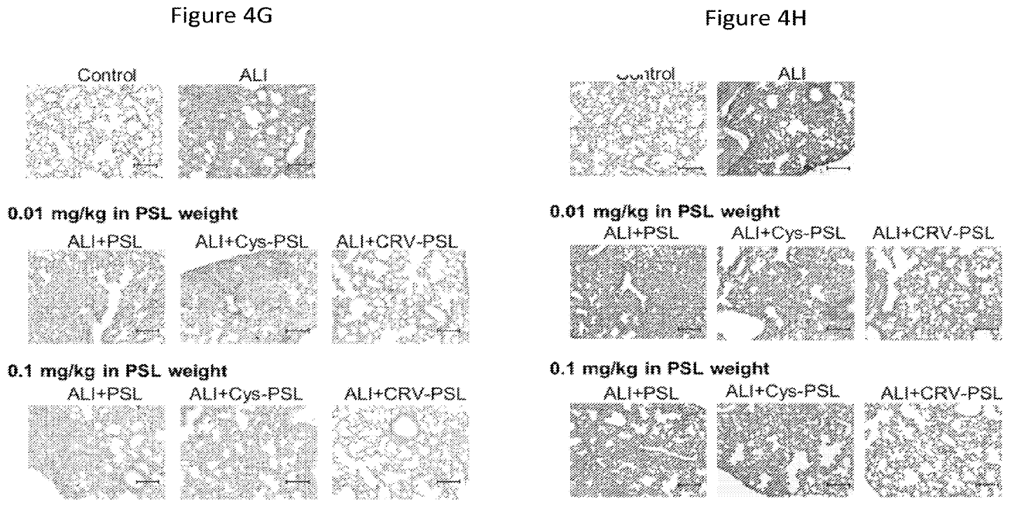

Next, we evaluated the impact of CRV conjugation on PSL efficacy to attenuate lung inflammation and injury (FIG. 4A). At day 4 post LPS stimulation, ALI lungs showed injury histology characterized by patchy areas of neutrophilic infiltration, alveolar hemorrhage, interstitial thickening, and lung consolidation) (FIG. 4B). ALI mice were treated with PSL, Cys-PSL, and CRV-PSL at a dosage of 0.01 mg kg⁻¹ and 0.1 mg kg⁻¹ (equivalent PSL weight) every 2 days for 4 days. At the end of treatment, hematoxylin and eosin (H&E) staining was per-formed on the lung tissue, and the lung injury score was quantified according to the changes of alveolar septae, alveolar hemorrhage, intra-alveolar fibrin, and intra-alveolar infiltrates as described by Matute-Bello et al. As shown in FIGS. 4B and 4C, CRV-PSL dis-played a stronger efficacy than PSL and Cys-PSL at both dosages to attenuate lung injury induced by LPS stimulation. Other than histology evaluation, we also examined the inflammatory responses in the lung by quantifying the percentage of infiltrated inflammatory cells in the lung as evaluated by CD64, expression of myeloperoxidase, and iNOS. While ALI lungs showed increased infiltration of inflammatory cells and elevated expression of inflammatory proteins compared with healthy control in agreement with previous reports, CRV-PSL treatment resulted in stronger suppression of these inflammatory markers than PSL and Cys-PSL (FIGS. 4D-41). Taken together, these results demonstrate that CRV conjugation increases the PSL efficacy to suppress inflammation and thus attenuates injury in ALI lungs.

### CRV Conjugation Reduces the Short-Term Side Effects of PSL in Healthy Organs

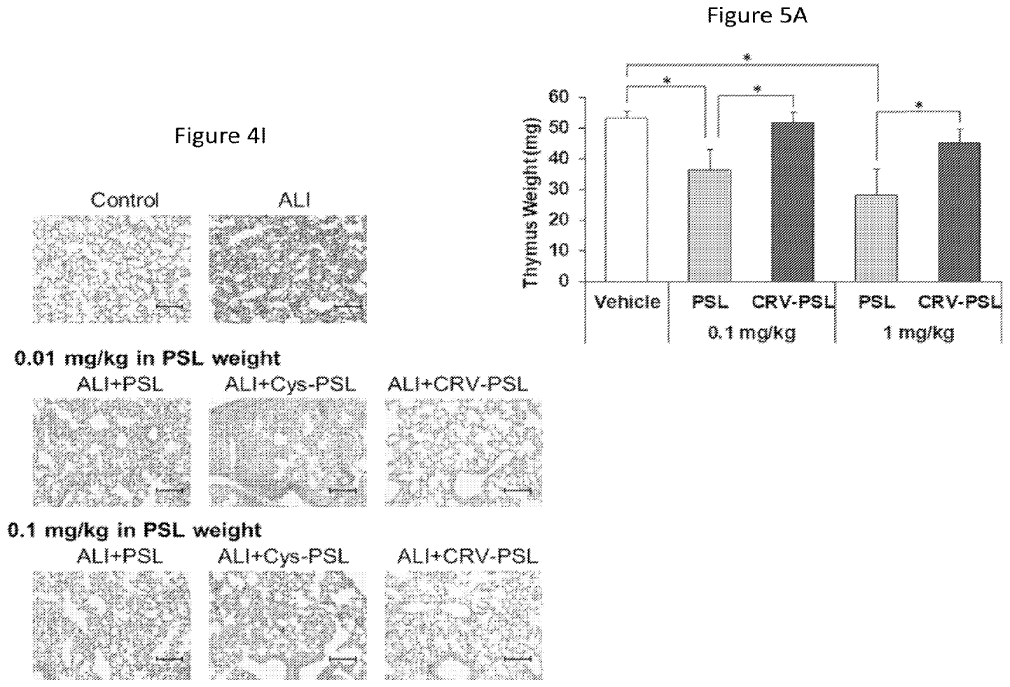

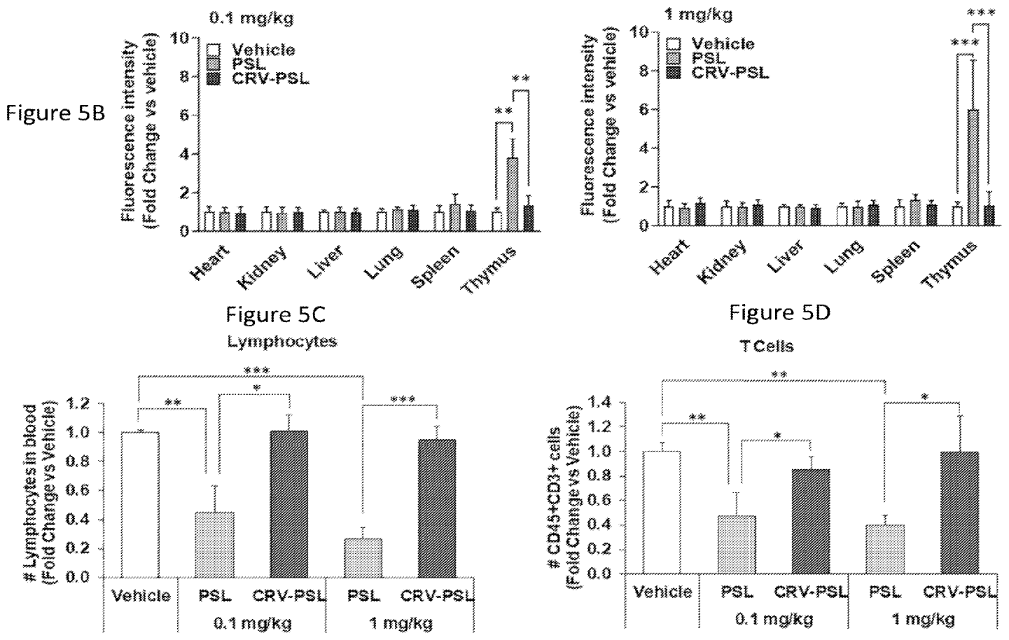

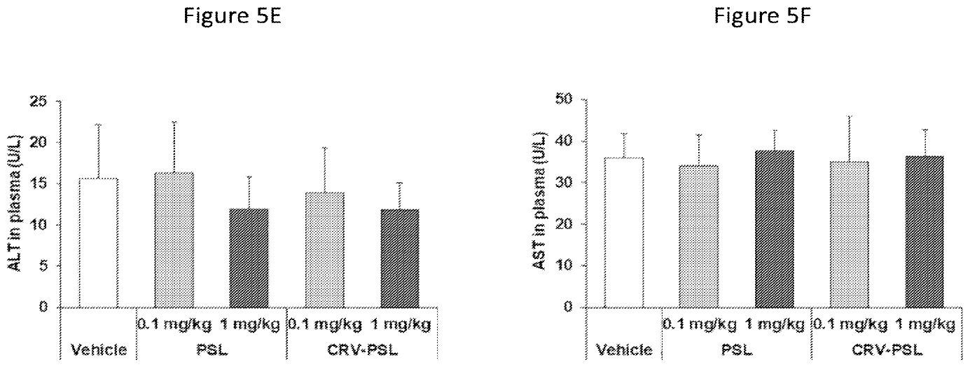

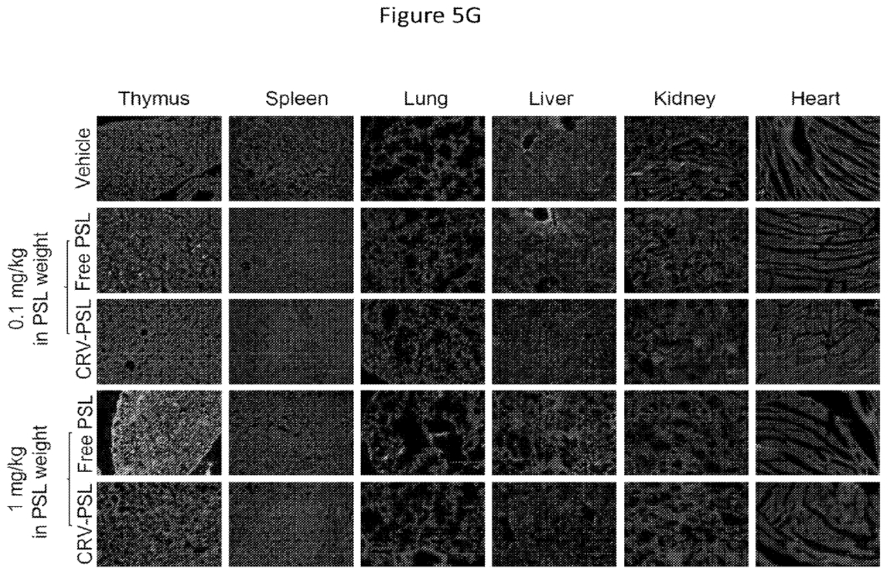

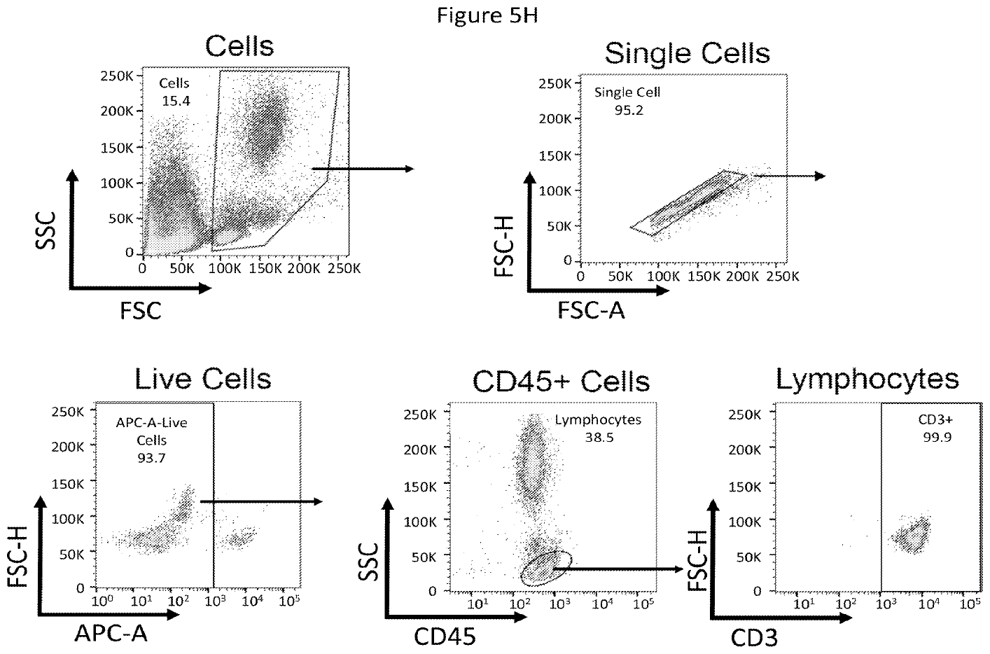

In this study, we mainly focused on the therapeutic benefits achieved after a short-term treatment (i.e., less than a week). Commonly seen side effects after a short-term exposure to glucocorticoid drugs include reduced thymus weight and lymphocyte count due to increased apoptosis. To evaluate the impact of CRV conjugation on the side effects of PSL, healthy mice were intravenously injected with PSL, Cys-PSL, or CRV-PSL every two days for five days with a dose of PSL equivalent to 0.1 or 1 mg kg⁻¹ of free PSL, which was lower than in previous reports. Four hours after the third injection on day 5, we collected various organs for analysis and used flow cytometry to quantify the total lymphocyte count and T cell subsets in circulation. While free PSL caused the greatest reduction in thymus weight, Cys conjugation rescued thymus weight to some extent and CRV conjugation was able to restore it back to nearly the weight in untreated animals (FIG. 5A, Table 4). We then performed TUNEL (terminal deoxynucleotidyl transferase dUTP nick end labeling) staining to quantify apoptosis in the thymus and other organs. The results of this analysis showed that PSL treatment increases the level of apoptosis mainly in the thymus, and CRV conjugation helps reduce this level back to the level of the untreated group (FIG. 5B, FIG. 5G).

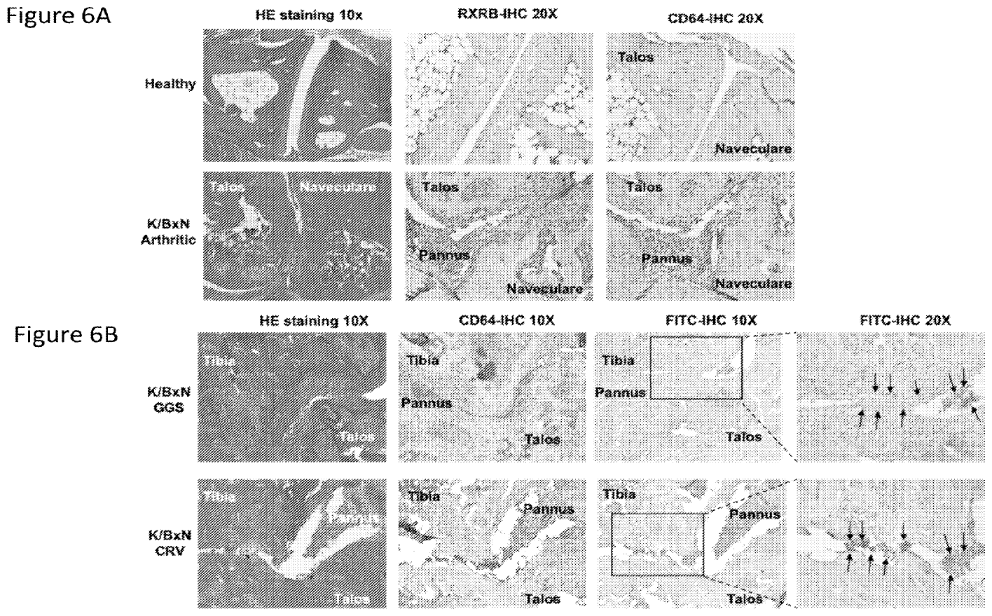

Similarly, we also quantified the toxicity towards lymphocytes. While PSL treatment reduced the counts of total lymphocytes and T subsets, CRV conjugation was able to restore it back to the level of the untreated group (FIG. 5C-D, Table 4, FIG. 6B). In addition to the above parameters, we also quantified the levels of the liver enzymes aspartate transaminase (AST) and alanine transaminase (ALT) in the blood, which showed no significant difference among all groups (FIG. 5E-F). Overall, these results support the notion that CRV conjugation attenuates the short-term side effects of PSL after systemic administration.

TABLE 4
Effect of CRV-PSL conjugate on thymus and T cell populations in ALI mice

| | | 0.1 mg kg⁻¹ PSL equivalent | | | 1 mg kg⁻¹ PSL equivalent | | |
|---|---|---|---|---|---|---|---|
| | Vehicle | Free PSL | Cys-PSL | CRV-PSL | Free PSL | Cys-PSL | CRV-PSL |
| Thymus | 53.3 ± | 36.3 ± | 44.5 ± | 51.9 ± | 28.3 ± | 34.3 ± | 45.0 ± |
| weight (mg) | 2.18 | 6.73* | 5.10*^{#} | 3.40^{#} | 8.46* | 3.65* | 4.62^{#&} |
| Lymphocytes | 1.00 ± | 0.45 ± | 0.70 ± | 1.02 ± | 0.27 ± | 0.35 ± | 0.95 ± |
| | 0.02 | 0.18** | 0.08* | 0.11^{#&} | 0.08*** | 0.17* | 0.09^{###&&} |
| T cells | 1.00 ± | 0.47 ± | 0.70 ± | 0.85 ± | 0.40 ± | 0.33 ± | 0.99 ± |
| | 0.07 | 0.19** | 0.09* | 0.10^{#} | 0.08** | 0.13* | 0.29^{#&} |

The count of total blood lymphocytes and T cells (CD45+CD3+) are presented as fold changes normalized to the vehicle group.
Values are expressed as mean ± SD (n = 3-4). Student's t-test performed for statistical analysis.
*P < 0.05,
**P < 0.01,
***P < 0.001 vs vehicle;
^{#}P < 0.05,
^{##}P < 0.01,
^{###}P < 0.001 vs free PSL;
^{&}P < 0.05,
^{&&}P < 0.01 vs Cys-PSL.
There is no statistical difference between any other groups.

The count of total blood lymphocytes and T cells (CD45+CD3+) are presented as fold changes normalized to the vehicle group.
Values are expressed as mean±SD (n=3-4). Student's t-test was performed for statistical analysis. *P<0.05, **P<0.01, ***P<0.001 vs vehicle; #P<0.05, ##P<0.01, ###P<0.001 vs free PSL; & P<0.05, && P<0.01 vs Cys-PSL. There is no statistical difference between any other groups.

### Discussion

In this study, we tested the ability of peptide-guided delivery to improve the therapeutic efficacy and safety of a glucocorticoid drug, PSL, for treating ALI. We found that the peptide CRV selectively accumulates in the inflamed lung of ALI mice upon intravenous injection, which may be attributed to the elevated expression of the receptor RXRB on the surface of myeloid infiltrates. In view of this targeting ability, we investigated whether conjugation to CRV would improve the biodistribution of PSL. Thus, CRV was covalently conjugated to PSL via a ROS-responsive linker. This conjugate remained intact in the plasma, but cleavage of PSL from the conjugate upon cell entry allowed this drug to exert its immunosuppressive activity in vitro at the similar level to native PSL. In vivo, CRV conjugation increased the accumulation of PSL in the lung of ALI mice, and thus enhanced its ability to reduce inflammation and rescue the lung injury. However, CRV conjugation reduced or had no impact on PSL accumulation in the healthy organs of ALI mice and lowered the acute side effects of PSL. Thus, CRV conjugation appears to improve the efficacy and safety of glucocorticoid drugs and can likely be applied to other drugs.

Myeloid-lineage immune cells play crucial roles in the pathogenesis of ALI. Upon injury/insult, pulmonary macrophages are activated to release cytokines and chemokines, leading to the recruitment of neutrophils and the alteration of alveolar membrane permeability followed by the destruction of alveolar structures and functions. Consistent with previous work, we found that neutrophils are the predominant immune infiltrates in the LPS-induced ALI model, followed by macrophages, and that lymphocytes account for only a very small portion of infiltrates. Upon LPS stimulation, overall expression of RXRB was elevated in the lung tissue. We also found that cell surface expression of RXRB was mainly elevated in the myeloid infiltrates of ALI lungs, which was not seen with control organs (blood and spleen), consistent with our previous study. The differential expression of RXRB, particularly at the cell surface, may at least partially explain the targeting specificity of CRV. The fact that CRV binds to different immune subsets upon intravenous injection further strengthens this speculation. The development of ALI depends on site-specific recruitment of neutrophils and monocytes from circulation, and these myeloid cells possess a high plasticity of gene expression in response to the local environment. Therefore, it is likely that RXRB expression in these infiltrating cells was increased only after they entered the lung tissue in response to inflammation. The exact role of RXRB in the biology of these immune cells and the pathophysiological functions of RXRB-positive cells remain to be further investigated.

To validate our findings in this murine model, we also examined patient specimens. In humans, ALI/ARDS can develop from various pulmonary disorders including ILDs. Myeloid infiltrates are present at high levels in the lung of ILD patients, and these levels are associated with disease severity. Besides confirming this result, we found that the overall level of RXRB is significantly increased in the lungs of ILD patients compared to healthy ones. This result demonstrates the clinical relevance of our study and may open new doors to understanding immune infiltrate subsets and immune dysfunction during the pathogenesis of ALI.

PSL and other glucocorticoids are important steroid drugs for treating a variety of autoimmune, allergic, and severe inflammatory disorders. While they have potent anti-inflammatory activities and broad clinical applications, glucocorticoids cause various side effects (e.g., protein catabolism, hyperglycemia, immunosuppression, infection, and osteoporosis) in both acute and long-term treatments, especially at high dosages. One reason that these drugs cause side effects is that they accumulate nonspecifically in healthy organs upon systemic administration, likely due to their hydrophobic nature. Several delivery strategies have been explored to improve the targeting specificity of these drugs. For example, nanoparticles such as liposomes have been used to encapsulate PSL and other glucocorticoid drugs. However, owing to their large size, a significant portion of these formulations were engulfed by organs of the reticuloendothelial system (RES) such as the liver and spleen. Glucocorticoid conjugates with peptides have been developed for treating rheumatoid arthritis, ocular diseases, and obesity, and drug modification with PEG was shown to increase the half-life and reduce nonspecific accumulation. Thus, instead of using CRV-pSiNPs to improve drug delivery, we instead directly conjugated CRV to PSL via a PEG linker. As a hydrophobic compound, PSL can diffuse freely through cellular membranes. Thus, in addition, an ROS-responsive linker was used to ensure that PSL can be released mainly after entry into target cells.

In agreement with previous reports, conjugation with PEG and CRV increased the plasma half-life and AUC of PSL. The ROS-responsive linker remained intact in the plasma of ALI mice and was cleaved upon H₂O₂ incubation. In vitro characterization was performed to verify that CRV-PSL possesses immunosuppressive activity. First, using LPS-induced inflammation in Raw264.7 cells, we confirmed that CRV-PSL exhibits an equivalent immunosuppressive activity to free PSL, which can only occur when PSL is cleaved off from PEG. Additionally, we detected the presence of PSL metabolites at similar levels after cells were incubated with PSL or CRV-PSL, which indicates that PSL is being cleaved from the conjugates. All these results support the notion that the CRV-PSL conjugate is stable until it is exposed to oxidative conditions inside inflammatory cells, at which time PSL can be released and thus exert its immunosuppressive function.

After in vitro characterization, we investigated the effect of CRV conjugation on PSL biodistribution in vivo and found that CRV conjugation increased the amount of both the whole conjugate and free PSL in ALI lungs but not healthy ones. CRV conjugation reduced the amount of free PSL in the liver and had little effect in other healthy organs. Overall, CRV conjugation prolonged the plasma half-life of PSL and shifted the drug biodistribution towards the target tissue (i.e., inflamed lung). Both of these effects may result in a better therapeutic outcome with fewer side effects. Therefore, we next evaluated the therapeutic efficacy of PSL conjugates against ALI.

Based on the intensity of lung injury and the expression of inflammatory markers, CRV-PSL showed the greatest ability to reduce lung inflammation and injury in ALI mice. Cys-PSL also showed a better therapeutic efficacy than free PSL, likely due to its prolonged half-life in the blood. We also tested the effect of CRV conjugation on side effects in healthy organs, which have been the main roadblock for clinical usage of glucocorticoids. We focused on acute side effects such as thymus and lymphocyte toxicity, which are relevant in the treatment ALI/ARDS. Our studies showed that while PSL causes toxicity, as demonstrated by decreased thymus weight and lymphocyte count, CRV-PSL reduces these side effects to untreated levels (FIG. 5 and Table 3). Cys-PSL also showed a reduced toxicity compared to PSL, likely due to reduced accumulation in healthy organs. Overall, our conjugates enable selective drug accumulation in disease sites and limit drug release to after the conjugate has entered target cells. Thus, CRV-PSL exhibits a stronger ability to attenuate inflammation and lung injury and nearly eliminates the acute side effects of PSL. In the future, this conjugation strategy may be applied to other drugs for ALI/ARDS treatment as well as to other diseases.

### Example 2: CRV-Dex for Treatment of Arthritis

In the following example, the inventors demonstrate that CRV can be used to target drugs to inflamed joints for treatment of arthritis.

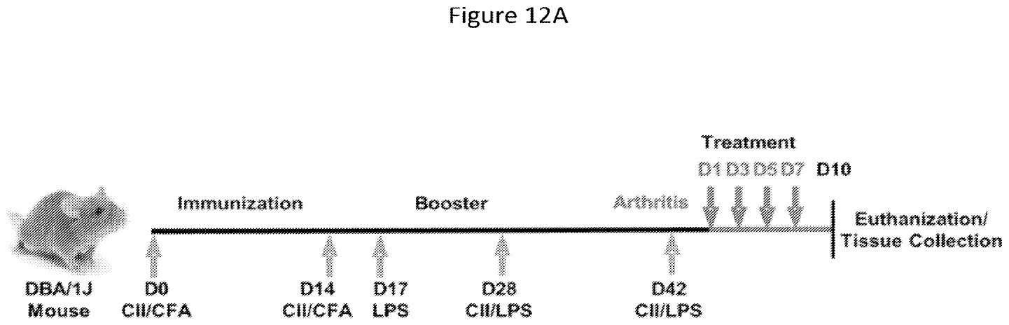

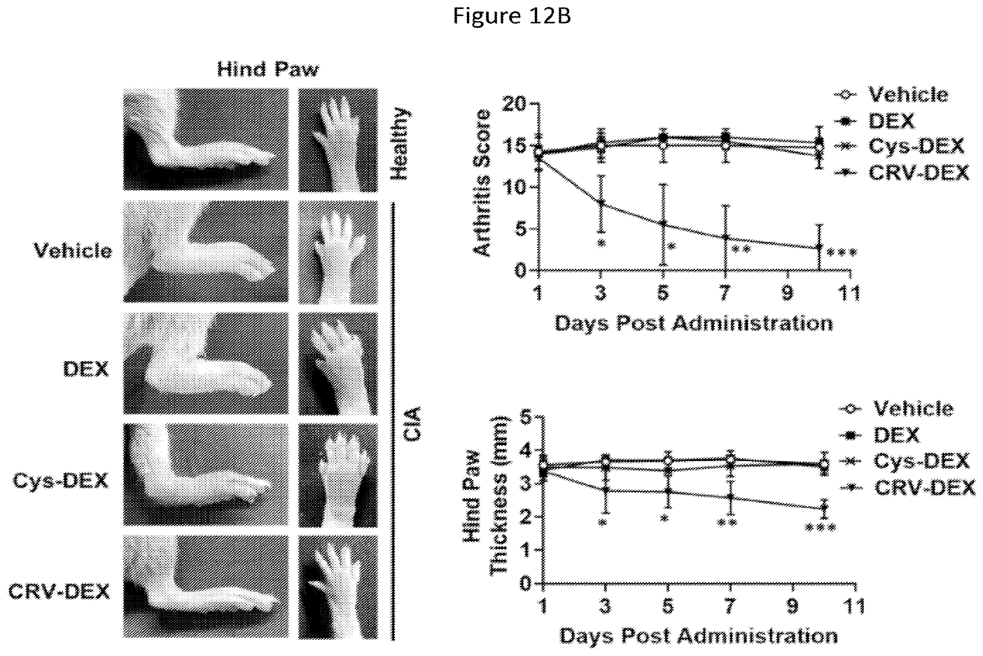

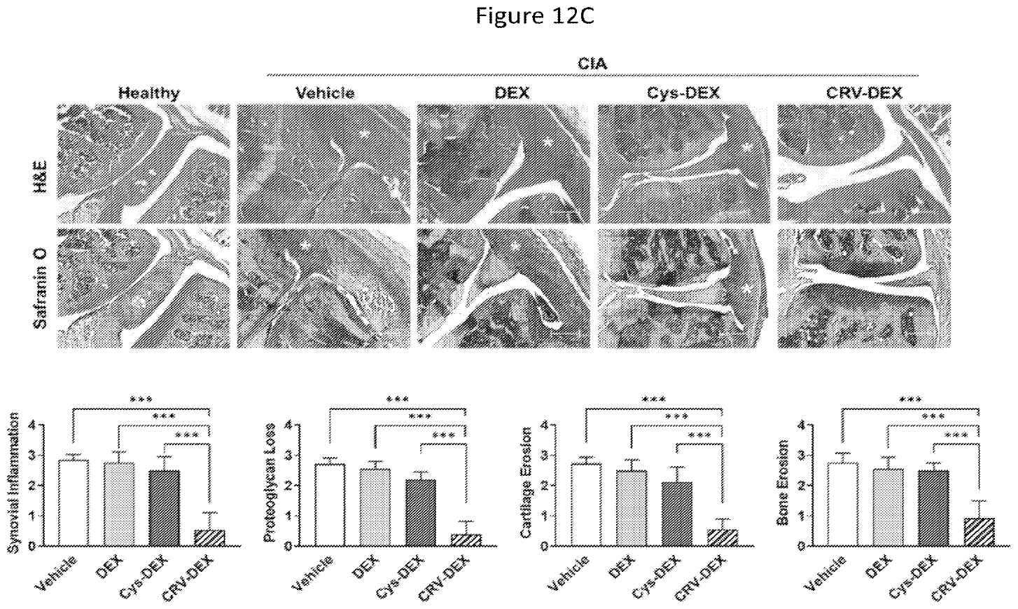

To evaluate the impact of CRV conjugation on the ability of the anti-inflammatory drug dexamethasone (DEX) to attenuate joint inflammation, mice with collagen-induced arthritis (an autoimmune model of rheumatoid arthritis; see Vet Pathol 52(5):819-26, 2015) were treated intravenously with free DEX or CRV-DEX at a dosage of DEX equivalent to 0.01 mg kg⁻¹ or 0.1 mg kg⁻¹ of free DEX by weight every two days for seven days. At both dosages, CRV-DEX displayed a stronger ability than DEX to attenuate paw swelling in arthritic mice (FIG. 12B).

Rheumatoid arthritis (RA) is a chronic inflammatory autoimmune disease that affects about 1% of the population worldwide. It is characterized by an excessive inflammatory response at synovium with infiltration of immune cells and pannus formation, which further causes cartilage and bone destruction and finally leads to significant joint deformity, disability, and a reduction of life quality. Although disease-modifying drugs have greatly improved the outcomes in early rheumatoid arthritis, more potent anti-inflammatory glucocorticoid drugs are still on the first line of treatment for advanced RA. However, long-term use or high dosage of glucocorticoids is associated with clinically significant toxic effects, including immunosuppression, hyperglycemia, and bone loss. Modified glucocorticoid therapies that more specifically target the desired site of inflammation with a minimally effective dose while reducing the unwanted systemic side effects need to be developed.

To overcome this obstacle, we developed a covalently conjugated dexamethasone (DEX), with a peptide that selectively targets the inflammation site of joints. Our group previously identified a cyclic peptide (CRVLRSGSC; SEQ ID NO: 1, termed “CRV” on the first three residues) and demonstrated its selective homing, upon intravenous injection, to the inflamed lung, but not healthy organs in a murine bacteria-induced lung infection model. We also applied the CRV-conjugated porous silicon nanoparticles (pSiNPs), which carried anti-inflammatory oligonucleotides, in the treatment of antibiotic-resistant lung infection. CRV conjugation significantly improved the drug delivery in the inflamed lung and achieved a higher efficacy of pSiNP-drug complex in lung infection. We have demonstrated the cell surface presence of retinoid X receptor beta (RXRB) on a subset of macrophages in the tumor tissues and on inflammatory myeloid cells in the inflamed lungs of acute lung injury model, which serves as the CRV receptor for targeting. Since macrophages and neutrophils play key roles in the pathogenesis of RA inflammation, we hypothesized that RXRB expression may change in synovial tissues of RA and the targeting peptide CRV may improve the drug delivery to the inflammatory synovium.

Our previous study has shown significant benefits of CRV conjugated glucocorticoid drug, prednisolone (PSL), in the treatment of lipopolysaccharide (LPS)-induced acute lung injury. In the CRV-PSL conjugation, a reactive oxygen species (ROS) responsive linker was used between CRV and PSL, which ensures the cleavage for drug release intracellularly under excessive ROS under inflammatory milieu. In this study, we generated CRV conjugated DEX and evaluated beneficial effects of the conjugation on the improvement of therapeutic efficacy and attenuation of long-term safety in treating rheumatoid arthritis.

### Materials & Methods

### Materials

Carboxyfluorescein-conjugated peptides<FAM>-<Ahx>-CRVLRSGSC (SEQ ID NO: 1) (FAM-CRV) and <FAM>-<Ahx>-GGSGGSKG (SEQ ID NO: 2) (FAM-GGS), <FAM>-Cys, <FAM>-Cys-<Ahx>-CRVLRSGSC (FAM-Cys-CRV), and PEG4-CRV were purchased from LifeTein (Somerset, NJ). Maleimide-PEG2000-Hydroxyl (HO-PEG2000-MAL) was purchased from Nanosoft Polymers (Winston-Salem, NC). Dexamethasone was purchased from Acros Organics (Carlsbad, CA). Sodium acetate, 2,2′-Thiodiacetic acid, N, N′-Dicyclohexylcarbodiimide (DCC), 4-Dimethylaminopyridine (DMAP), N, N-Dimethylformamide (DMF), Ethylenediaminetetraacetic acid (EDTA), and RPMI-1640 Medium were purchased from Sigma-Aldrich (St Louis, MO). Fetal bovine serum (FBS) and DOTA-NHS-ester were from Thermo Fisher Scientific (Waltham, MA). ⁶⁴Cu was purchased from University of Wisconsin-Madison. Human synovial total RNA from healthy and the patients with rheumatoid arthritis was purchased from Origene (Rockville, MD).

### Synthesis of CRV-DEX Conjugates

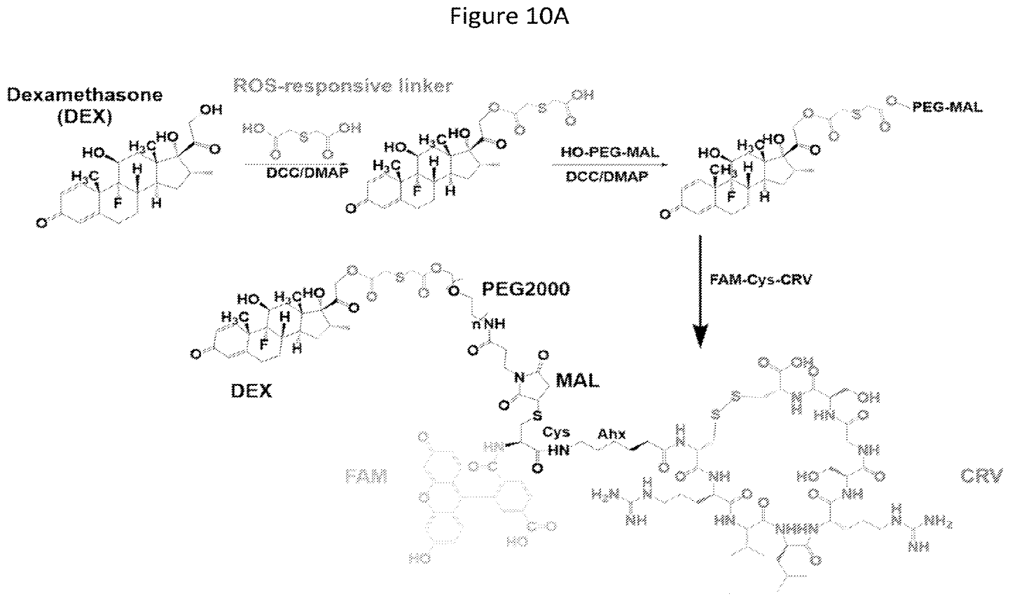

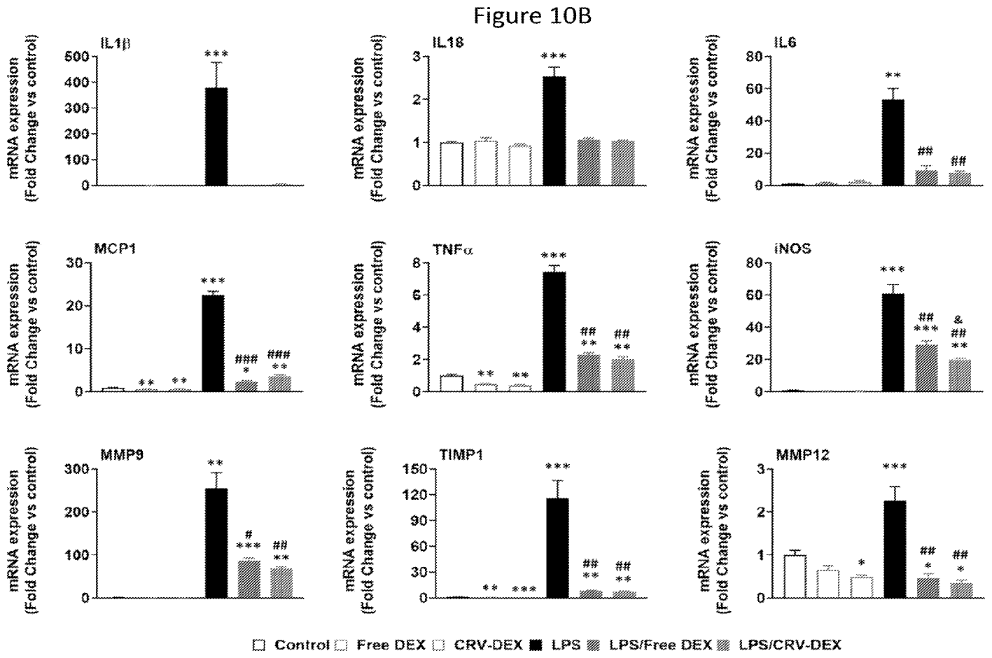

FAM-Cys-CRV-DEX (FAM-CRV-DEX) and FAM-Cys-DEX were designed and synthesized as shown in FIGS. 7A and 10A. In brief, equimolar of DEX and 2,2′-thiodiacetic acid were solved in DMF with magnetic stirring under 80° C. followed by adding the solution of 1.5 equivalents of DMAP and 3 equivalents of DCC in DMF to construct ROS-responsive drug delivery system. After overnight reaction and removal of DCC derived urea by a cellulose/cotton filter, the product was purified by a preparative HPLC (Mobile phase: acetonitrile (ACN) & water, Gradient elution: 10% ACN→90% ACN, Column: Luna© 5 m 250*10 mm C18). The dried product was then mixed with equimolar of HO-PEG2000-MAL and solved in DMF followed by adding the solution of 1.5 equivalents of DMAP and 3 equivalents of DCC in DMF with magnetic stirring at room temperature. After 2-h reaction, the DCC derived urea was removed by a cellulose/cotton filter. The product was precipitated by 30-40 times the volume of ethanol and diethyl ether and then was dissolved in PBS with 10% DMF to mix with 3 equivalents of FAM-Cys or FAM-Cys-CRV. The carboxyfluorescein dye FAM was attached through the linker to the N-terminus of CRV peptide for tracking. After 2 h reaction, the finial product (prodrug) was purified with a 2 KDa dialysis bag and dried by lyophilization.

### Cell Culture

RAW 264.7 cells were purchased from American Type Culture Collection (ATCC, Manassas, VA). The cells were cultured in RPMI-1640 medium containing 50 U per mL streptomycin, 100 U per mL penicillin, and 10% FBS at 37° C. in a humidified incubator with 5% CO₂. All the cell cultures were maintained in 6-well culture plate for different treatments.

### Animals

DBA/1J mice were purchased from The Jackson Laboratory (Bar Harbor, ME) and were housed in a specific pathogen-free facility with free access to food and water at the Research Animal Resources (RAR) facility of the University of Minnesota. All animal studies were carried out in compliance with the National Institutes of Health guidelines and an approved protocol from University of Minnesota (UMN) Animal Care and Use Committee.

### Establishment of CIA Model

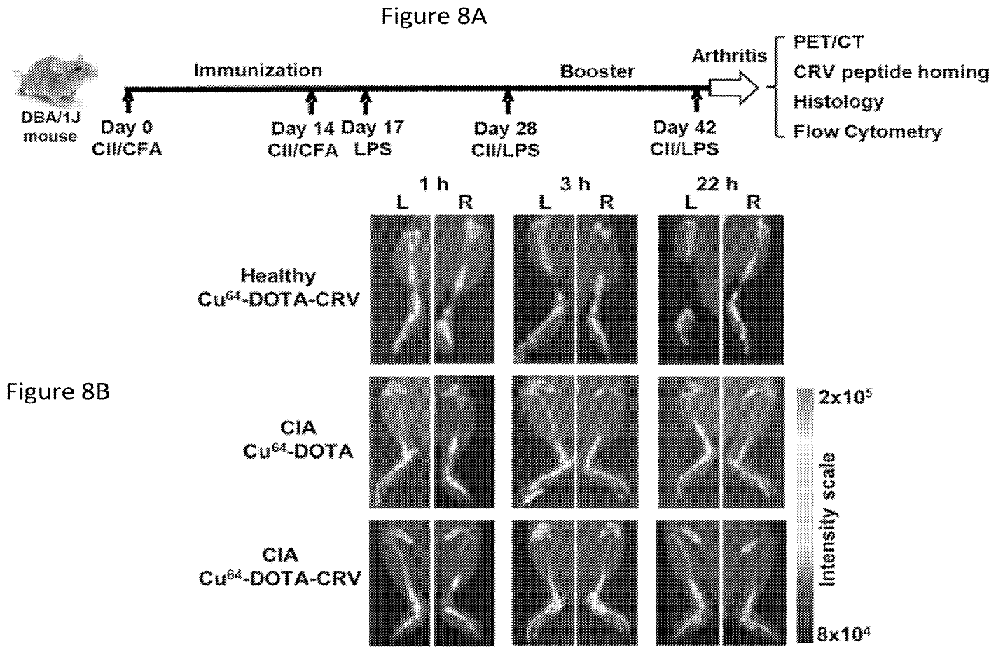

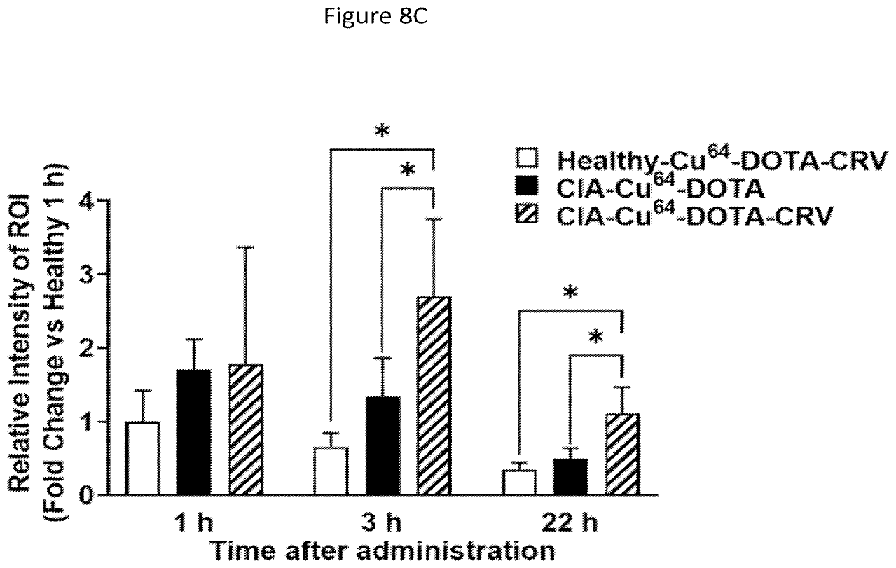

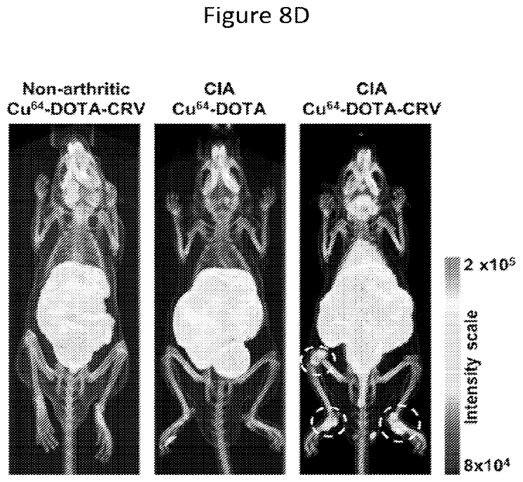

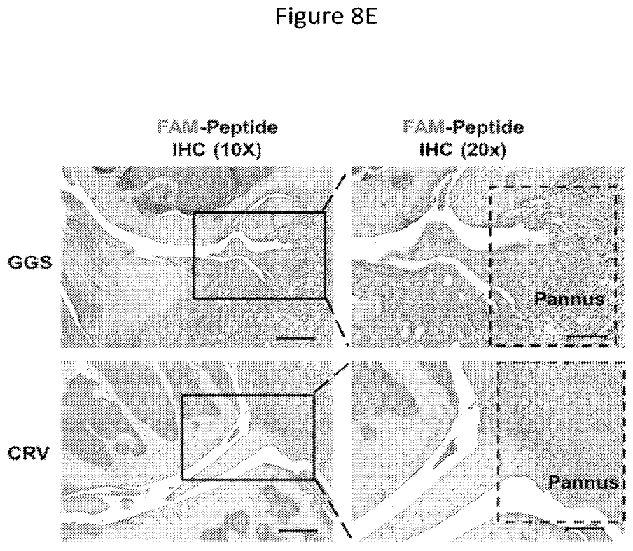

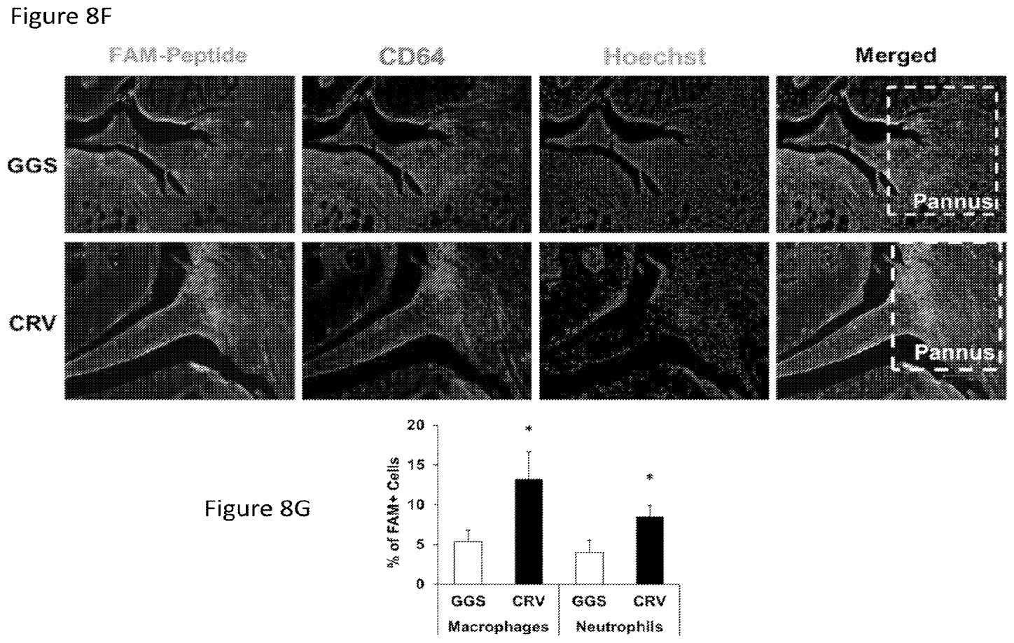

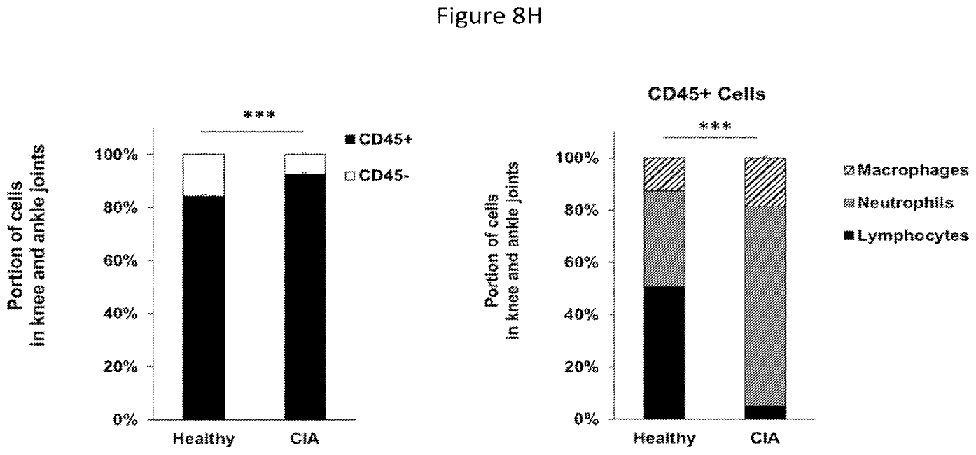

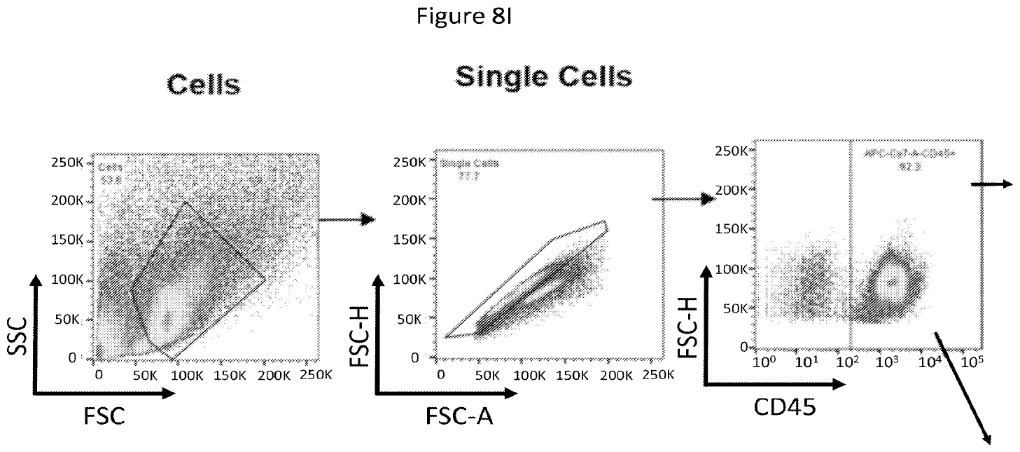

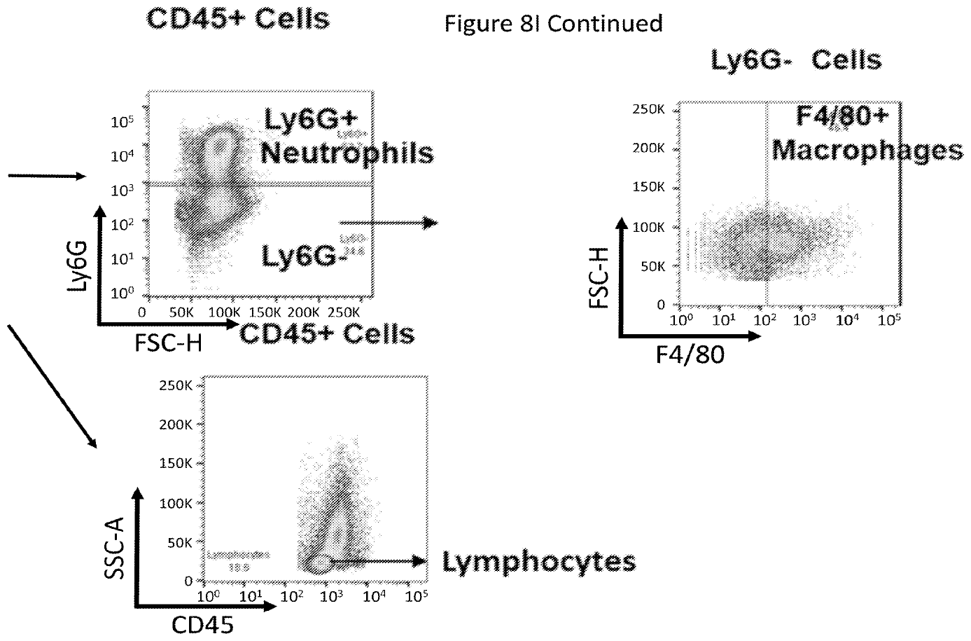

The collagen-induced arthritis model was established as previously described. Briefly, chicken collagen type II (CII, Chondrex, Woodinville, WA) was dissolved in 0.05 M acetic acid as a 2 mg/mL solution by gently stirring overnight at 4° C. and then emulsified with equal volume of complete Freund's adjuvant (CFA, Sigma-Aldrich) on ice. To induce arthritis, DBA/1J male mice at 6-7 weeks of age were given intradermal injection of 0.1 mL (100 g collagen/mouse) of the emulsified solution for immunization on day 0 and day 14. After intradermal injection of 50 μg of LPS (from Escherichia coli 0111:B4, Sigma) on day 17, the mice were given intradermally two doses of CII/LPS solution with a final ratio of 100 μg CII and 50 μg LPS in a final volume of 100 μL on day 28 and day 42. The scheme of the establishment of CIA model is shown in FIG. 8A.

### Synthesis of ⁶⁴Cu-DOTA-CRV

The synthesis of ⁶⁴Cu-DOTA-CRV was adapted from a previous protocol. The scheme of synthesis is shown in FIG. 7B. Equal molars of DOTA-NHS and PEG4-CRV were dissolved in normal saline (0.9% sodium chloride) to form DOTA-PEG4-CRV (DOTA-CRV) with a final concentration of 2 mg/mL. DOTA-NHS ester (DOTA) served as the control. The ⁶⁴Cu received from the University of Wisconsin-Madison was immediately diluted with sodium acetate buffer (0.1 M pH 5.5) to 20 μCi/L. Fifty microliters of ⁶⁴Cu solution will be added to 50 μL DOTA-CRV or DOTA solution in normal saline to 500 μCi per mouse. The mixture was incubated at 55° C. for 50 min to 1 h. Free ⁶⁴Cu was separated using Pierce Polyacrylamide desalting columns and spinning at 1500 g for 2 min in a minicentrifuge. Recovered conjugated ⁶⁴Cu-DOTA-CRV or 64Cu-DOTA was brought up to a final volume of 200 μL with normal saline for use.

### PET/CT Scan

PET/CT scanning was performed using an Sofie G8 PET/CT imaging system (Sofie, Dulles, VA). The mice were given 100 μL of ⁶⁴Cu-DOTA-CRV or ⁶⁴Cu-DOTA intravenously via retro orbital injection under an anesthetic condition. For imaging, a static 20-min emission scan was acquired followed by a micro-CT at 1 h, 3 h, and 22 h of post-injection. PET images were reconstructed and analyzed using Amide software (amide.sourceforge.net). Quantification of PET signals were performed by drawing spherical regions of interest (ROI) around knee and ankle, forming four ROIs per mouse. The ⁶⁴Cu uptake was quantified by the intensity of emission using equal ROI size for knees and ankle joints to generate a total uptake value in all groups of mice at different time points.

### HPLC-MS/MS Assay

HPLC-MS/MS analysis was performed using the chromatographic system consisting of UltiMate 3000 RSLCnano System (Thermo Fisher Scientific) and a ZORBAX C18 column (5 μm, 150 mm 0.5 mm, Agilent, Santa Clara, CA). The mobile phase was a mixture of H₂O/acetonitrile with a gradient elution (from 90:10 to 10:90 and return to 90:10, v/v) at a flow rate of 15 μl/min. The eluent was introduced directly into the electrospray source of a tandem quadrupole mass spectrometer (TSQ VANTAGE, Thermo Fisher Scientific) that was operated in the positive mode. The spray voltage was set at 3000 V. The compounds were analyzed by multiple reaction monitoring (MRM) of the transitions of m/z (ESI+) 361.24→147.06 for PSL.

### In Vivo Pharmacokinetics Assay

Healthy DBA/1J male mice at age of 8-9 weeks were deprived of food overnight with free access to water. The mice were given intravenously free DEX, Cys-DEX, and CRV-DEX with an equivalent dose of 1 mg kg⁻¹ DEX, respectively. Approximately 20 IL of blood was collected via facial vein at 5, 15, 30, 60 min and, 2, 6, 12, 24, and 48 h, and plasma were obtained by centrifugation at 500×g for 10 min. Ten microliters of 0.1 M sodium hydroxide were added and vortexed for 30 seconds and then mixed with 200 μl of methanol/DCM (1:1) by vortex for 1 min. After centrifugation at 16000 g for 10 min, the upper liquid layer was collected, blown-dry by nitrogen, and dissolved in 100 μl ACN for HPLC-MS/MS analysis.

### In Vivo Drug Distribution

Once arthritis developed, the mice were randomly divided into three groups and were given free DEX, Cys-DEX, or CRV-DEX with an equivalent dose of DEX at 1 mg kg⁻¹ via intravenous injection for 3 h. After transcardial perfusion, the tissues including the knee and ankle joints, thymus, brain, heart, lung, liver, spleen, and kidney were harvested and weighted. The tissues were then homogenized and dissolved in 200-500 μl of methanol/DCM (1.1) for 1 min vortex and centrifugation at 16000 g for 10 min. The upper layer was taken and blown dry by nitrogen. The sample was then dissolved in 100 μl of ACN for HPLC-MS/MS assay.

### Real-Time RT-PCR

Gene expression in cells and tissues was determined by real-time reverse transcription polymerase chain reaction (RT-PCR). In brief, after total RNA extraction by TRI reagent (Sigma-Aldrich), first-strand cDNA was synthesized from total RNA using an iScript™ cDNA Synthesis Kit (Bio-Rad Laboratories, Hercules, CA). Quantitative amplification of PCR product was performed using PowerUp SYBR Green qPCR Master Mix (Thermo Fisher Scientific) by a StepOne Real-time PCR System (Applied Biosystem, Foster City, CA). The ΔΔCt method was used to calculate the results. The house keeping genes were used as endogenous controls for quantification. The sequences for the relevant primers are listed in Table 1.

### Flow Cytometry

The single cells were isolated from joint tissues using a tissue dissociation kit for mouse (Miltenyi Biotec, Auburn, CA). To Identify subpopulations in myeloid cells, the isolated single-cell suspension with 0.5×10⁶ cells were stained with fluorophore-conjugated antibodies as indicated in the results section and in Table 2. Flow cytometry analysis was performed on BD Fortessa™ X-20 (BD Biosciences, East Rutherford, NJ) and the data were analyzed with Flow Jo software (Tree Star, San Carlos, CA).

Histology Analysis with Hematoxylin-Eosin (H&E) and Safranin O/Fast Green Staining

Joints and paws selected for histology were fixed in 10% neutral buffered formalin followed by decalcification with 14% EDTA for at least 2 weeks. The tissues were further processed to coronal hemisections for the knee and sagittal hemisections representing ankle joints. H&E and safranin O/Fast green staining was performed on the paraffin-embedded sections with 5 μm of thickness according to standard procedures. The synovial inflammation score was evaluated in H&E staining sections by an independent investigator in a blinded manner according to the assessment methods described previously. In brief, the score is graded from 0 with 1-2 layers of synovial membrane, no inflammatory infiltrate to score 3 with severe hyperplasia of synovial membranes and massive accumulation of inflammatory cells throughout entire synovial membranes and connective tissues. Bone erosion score was also evaluated by H&E stained sections, grading from score 0 with intact bone surface, score 1 with small and superficial bone erosion at outer surface of bone, score 2 with enhanced focal bone erosion with partial or complete penetration of cortical bone, to score 3 with massive, enlarged subchondral bone erosion, extended synovial pannus invasion, causing complete breakthrough of the cortical bone and loss of bone structure. The cartilage damage was evaluated by cartilage erosion score and loss of proteoglycan with safranin O/Fast green staining. The loss of proteoglycan, the major component of cartilage, was scaled from score 0 defined as no loss of proteoglycans to score 3 with complete loss of staining for proteoglycans. The score of cartilage erosion was defined as score 0 with intact articular cartilage to score 3 with erosion of the superficial cartilage layer and/or destruction of the underlying calcified cartilage layer by the invasion of pannus tissues.

### Immunohistochemistry (IHC) Staining

Immunohistochemical staining was performed on the paraffin-embedded sections. In brief, the sections were deparaffinized, rehydrated, and then incubated with 0.3% H₂O₂ solution for blockade of endogenous peroxidase activity. After blocking with 5% donkey serum Hocking solution (with 0.1% triton X100), the sections were incubated with primary antibodies overnight at 4° C. The primary antibodies included polyclonal rabbit anti-RXRB (GeneTex, Irvine, CA), anti-fluorescein/Oregon Green (Invitrogen, Waltham, MA), anti-NOS (PA1-036) and anti-myeloperoxidase antibody (PA5-16672, Thermo Fisher Scientific); and rat CD64 (Thermo Fisher Scientific). The sections were then treated with secondary anti-rabbit- or anti-rat-HRP antibody for 1 b followed by DAB peroxidase (HRP) substrate (Vecto. Labs, Burlingame, CA) for 1-2 mir Hematoxylin counterstaining was performed to stain nucleus followed by dehydration in ethanol and xylene and mounting with Permount (Thermo Fisher Scientific). Images of knee and ankle joints were captured from each section. The expression of the targeted proteins was semi-quantified as described previously. In brief, the percentage of positive cells was graded as score 0=negative; score 1=0-25%; score 3=50-75%; score 4: 76-100%. The staining intensity was defined as score 0 to 3 (from no signal, weak, moderate to intensive) according to the HRP-DAB signal. The final score was obtained by multiplying the percentage score and staining intensity score.

### Immunofluorescence (IF) Staining

Immunofluorescence staining was performed on the paraffin-embedded sections. In brief, after deparaffinization and rehydration, the sections were incubated with PBS containing 1% BSA and 0.1% Triton X100 (blocking buffer) at room temperature (RT) for 1 h. The sections were then washed three times with PBS followed by the incubation with primary antibodies with a 1:200 dilution in blocking buffer at 4° C. overnight. The primary antibodies included rabbit anti-fluorescein/Oregon Green (Invitrogen); anti-RXRB (GeneTex), and rat anti-CD64 (Thermo Fisher Scientific). The sections were then stained with appropriate secondary antibodies diluted (1:200) in blocking buffer at RT for 1 h. After washing with PBS, the sections were stained with Hoechst 33342 Solution (Thermo Fisher Scientific) for nuclei. The sections were examined under fluorescence microscope EVOS M5000 (Thermo Fisher Scientific).

### TUNEL Staining

The TUNEL staining was performed using an In Situ Cell Death Detection Kit (Roche Diagnostics GmbH, Mannheim, Germany). The paraffin sections were stained with TUNEL reaction mixture for 1 h at 37° C. followed by Hoechst staining for nucleus. The images were captured by using fluorescence microscopy EVOS M5000 and the fluorescence intensity of TUNEL positive cells was evaluated by ImageJ software for semi-quantification.

### Biochemical Toxicity Analysis

The aminotransferase quantification was performed to determine toxicity effect of different formulations of PSL on the liver. Blood samples were collected by cardiac puncture after the treatment. The levels of aspartate aminotransferase (AST) and alanine aminotransferase (ALT) in plasma were measured using assay kits from Stanbio (Boerne, TX).

### Statistical Analysis

A test of normal distribution of the variants was performed showing that the variants were normally distributed before one-way analysis of variance (ANOVA) or Student's t-test, where appropriate to assess differences among groups. The gene expression by qPCR were expressed as means±standard error of mean (SEM). Other results were expressed as mean±standard deviation (SD). A P value less than 0.05 was considered significant.

### Results

### CRV Accumulates in Arthritic Joints

To study the accumulation of CRV in inflamed joints, 100 μl of fluorescein (FAM)-CRV peptide solution (1 mg/ml in PBS) and the control peptide, GGS (SEQ ID NO: 2), was injected intravenously into mice with K/B×N serum-transfer arthritis (for a description of this mouse model, see VetPathol 52(5):819-26, 2015). The ankle joints were harvested 1 hour after injection. Immunohistochemistry (IHC) staining showed that the expression of the CRV receptor RXRB is significantly upregulated in arthritic joints as compared to in healthy controls (FIG. 6A). IHC staining (i.e., using a FITC antibody for FAM detection) also showed that CRV exhibited much higher accumulation in the arthritic joints than the control peptide GGS (FIG. 6B).

### CRV Targets Synovial Inflammation Sites of Arthritic Joints Upon Systemic Administration

Using a widely used collagen-induced arthritis mouse model to mimic human rheumatoid arthritis (FIG. 8A), we examined the distribution of radiolabeled CRV in the extremity joints of arthritic mice by positron emission tomography/computed tomography (PET/CT) imaging. CRV was first conjugated to DOTA (1,4,7,10-tetraazacyclododocane tetraacetic acid) and then radiolabeled with copper 64 (⁶⁴Cu) to form ⁶⁴Cu-DOTA-CRV by adapting methods reported previously. The synthetic scheme is shown in FIG. 7B. Once arthritis developed, ⁶⁴Cu-DOTA-CRV were given intravenously into the mice for 1 h, 3 h, and 22 h homing. PET/CT imaging and the radioactivity quantification showed that ⁶⁴Cu-DOTA-CRV significantly accumulate at the knee and ankle joints within 3 h with a retention until 22 h post-injection, compared to the ⁶⁴Cu-DOTA group and the healthy controls receiving ⁶⁴Cu-DOTA-CRV (FIG. 8B-C). ⁶⁴Cu-DOTA-CRV were also observed in other healthy organs at 3 h with the strongest signals in the liver, suggesting the hepatic clearance of the peptides at this time point (FIG. 8D).

These were further confirmed by the intravenous injection of fluorescein (FAM) labeled CRV into the mice with arthritis for 1 h homing. Immunohistochemistry (IHC) showed that CRV accumulates in the inflammatory synovial tissues (pannus) of joints at a markedly higher level than a control peptide, GGS (FIG. 8E). There was some accumulation of CRV in the liver and kidney, while no or much less in other organs (FIG. 7C). By immunofluorescence (IF), CRV predominately colocalizes with CD64-positive immune cells in the inflamed synovium of arthritic joint, but this was not seen in GGS group (FIG. 8F). Similar results were also found in another model of human inflammatory arthritis, K/B×N serum-transfer arthritis, showing significant CRV accumulation with increased inflammatory cells in arthritic joints, while few FAM signals were detected in GGS control (FIG. 7D).

To further dissect the cell types of CRV targeting in inflammatory synovium, single cells from healthy and CIA joint tissues were isolated and sorted with CD45 for leucocytes, Ly6G for neutrophils, and F4/80 for macrophages. The percentages of neutrophils and macrophages were significantly increased in the CIA synovial tissues compared to healthy ones, as shown in FIGS. 8H-I, agreeing with previous reports. Meanwhile, the content of CRV in different immune subsets of CIA joint were examined after in vitro incubation with the peptides for 1 h at 4° C. Corroborating with the above results, flow cytometry analysis showed that CRV exhibits more binding to macrophages and neutrophils than GGS controls as evaluated by the percentage of FAM positive signals, indicating a preferential binding ability of CRV (FIG. 8G). Taken together, CRV mainly targets the inflamed synovial tissues in the arthritic joints, which suggests the targeting potential of CRV to inflammatory cells at pannus of arthritis.

### CRV Targeting is Due to Increased Expression of RXRB

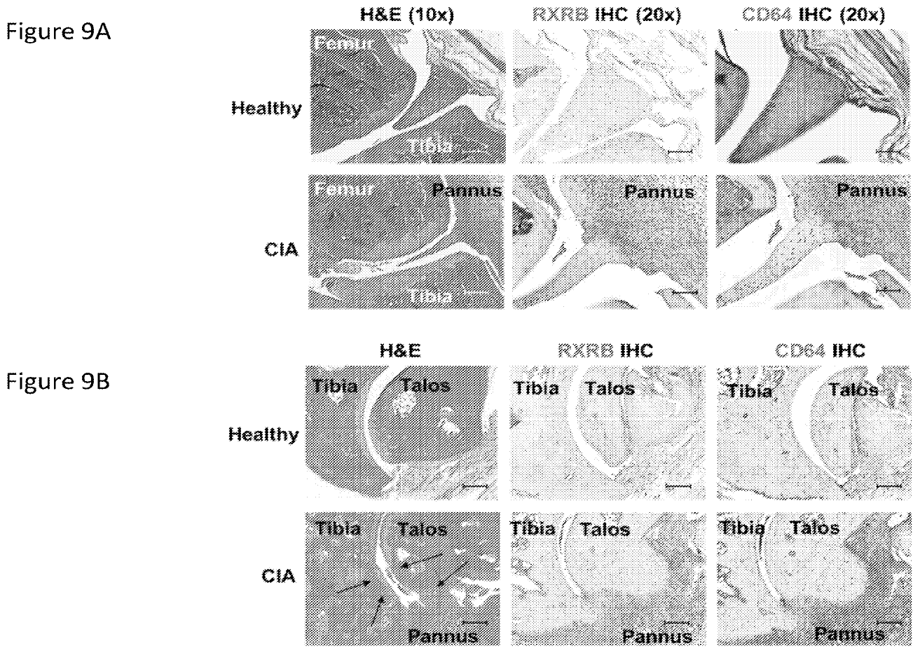

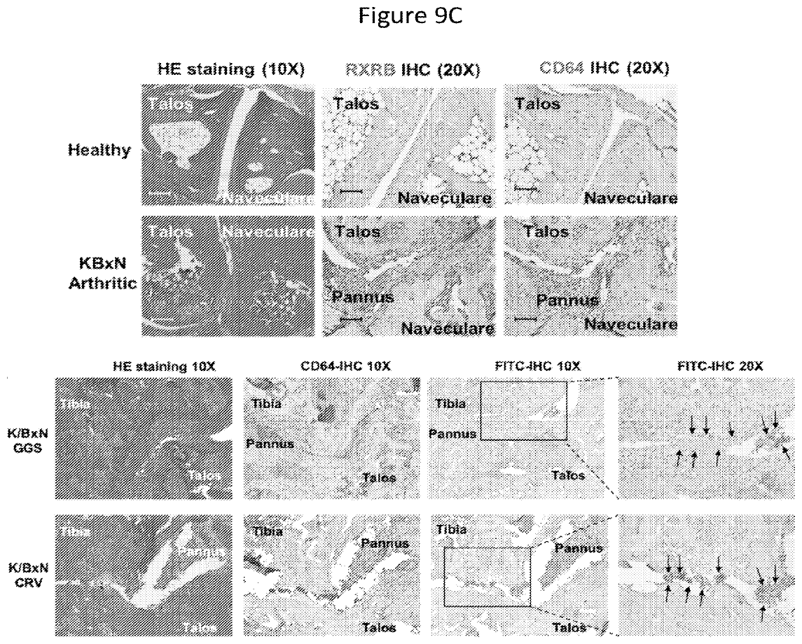

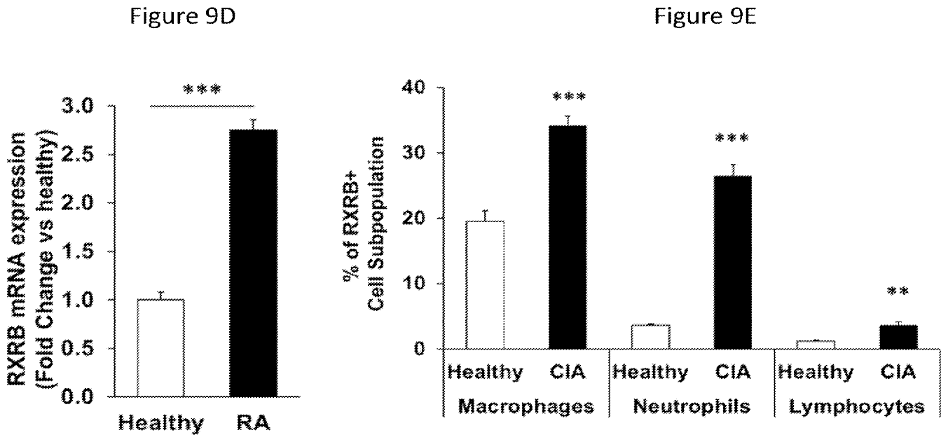

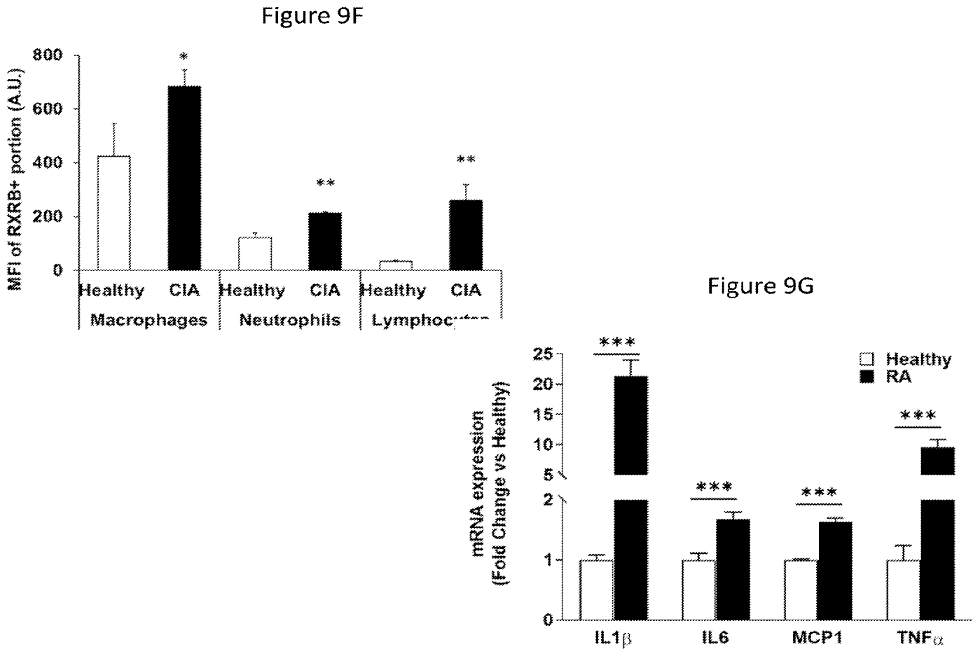

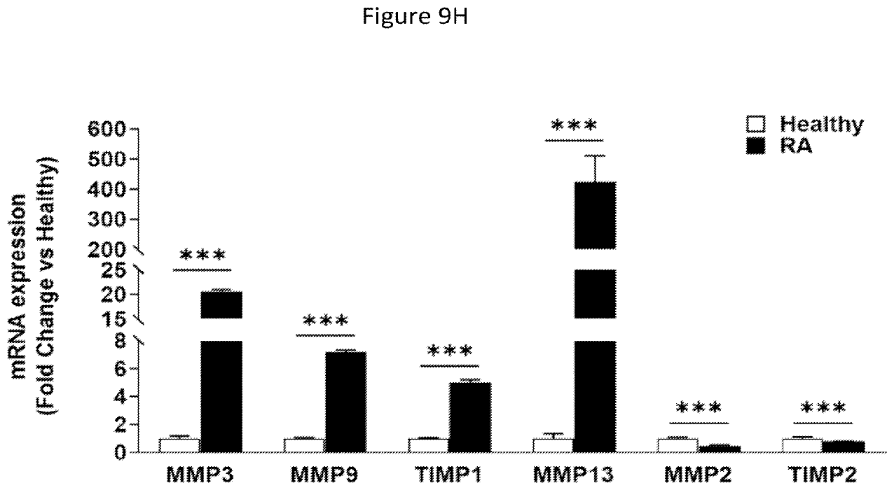

Our previous studies have shown that RXRB is highly expressed on the surface of inflammatory macrophages, which serves as the receptor of CRV. To further understand the basis of CRV targeting, we investigated the expression of RXRB expression in inflammatory joints of arthritis models. We found markedly increased expression of RXRB at the inflammatory synovial tissues (pannus) in the joints CIA mice, which colocalized with inflammatory cells as stained with CD64 (FIG. 9A-B). This was also observed in the K/B×N serum-transfer arthritic model (FIG. 9C; top). IHC staining (i.e., using a FITC antibody for FAM detection) also showed that CRV exhibited much higher accumulation in the arthritic joints than the control peptide GGS (FIG. 9C; bottom). Besides the murine models, we detected the gene expression of RXRB in synovial tissues from the patients with rheumatoid arthritis. The expression of RXRB was significantly upregulated in the synovium of RA patients, compared to the healthy controls (FIG. 9D). In accordance with the increased RXRB, we also observed the upregulated expression of inflammatory genes including interleukin 1 beta (IL1β), IL6, tumor necrosis factor alpha (TNFα), and monocyte chemoattractant protein-1 (MCP-1) as well as the genes related with extracellular matrix (ECM) formation at arthritic joints such as matrix metalloproteinase (MMP) 2, MMP3, MMP9, MMP13, tissue inhibitor of metalloproteinases (TIMP) 1, and TIMP2 in the arthritic synovium of RA patients (FIG. 9G-H).

To show the surface expression of RXRB in inflammatory cells of arthritic joints, the single cells from healthy and arthritic joints were further sorted with neutrophils (Ly6G+), macrophages (Ly6G-F4/80+), and lymphocytes for flow cytometry (FIG. 8H-I). The portion of RXRB-positive cells was significantly elevated in the inflammatory cells of arthritic joints than healthy ones (FIG. 9E-F). Among these inflammatory cells, macrophages exhibited the highest expression of RXRB, indicating a potential of CRV targeting.

### Synthesis and In Vitro Characterization of CRV-Drug Conjugate

To investigate whether CRV can improve the delivery of glucocorticoid drugs to the inflamed joint, CRV-conjugated DEX (CRV-DEX) was synthesized by adapting the method described previously and the procedures were schemed in FIG. 7A. The control conjugate without targeting peptide was generated by replacing CRV with a single Cysteine, termed Cys-DEX (FIG. 7A). The ROS-responsive linker is expected to be cleaved since oxidative stress is one of the important drivers for chronic inflammation in the joints, and hence free DEX will be released from the conjugation. We further validated that the majority of CRV-DEX remains stable and uncleaved in the plasma from CIA mice at least for 24 hours. This indicated that the ROS-responsive conjugation is stable in the circulation and allows the release of free DEX under oxidative condition at the inflamed synovial tissues of arthritic joints.

With the polyethylene glycol (PEG) conjugation that may render the corticosteroids inactive until cleaved, CRV-DEX is expected to become active and subject to release free DEX under inflammatory milieu. Therefore, we verified whether CRV-DEX has the equivalent immunosuppressive activity to DEX using a RAW 264.7 macrophage cell line in response to increased ROS production by LPS stimulation. After 18 h of treatment, LPS induced significant inflammatory responses as examined by the expression of proinflammatory markers including interleukin (IL) 1P, IL18, IL6, MCP-1, TNFα, and inducible nitric oxide synthase (iNOS); and the genes associated with ECM formation in arthritis such as MMP9, MMP12, and TIMP1. CRV-DEX exhibited similar or even stronger suppressive activity than free DEX on LPS-induced inflammation (FIG. 10B).

### CRV Conjugation Improves the Drug Biodistribution Towards Arthritic Joints

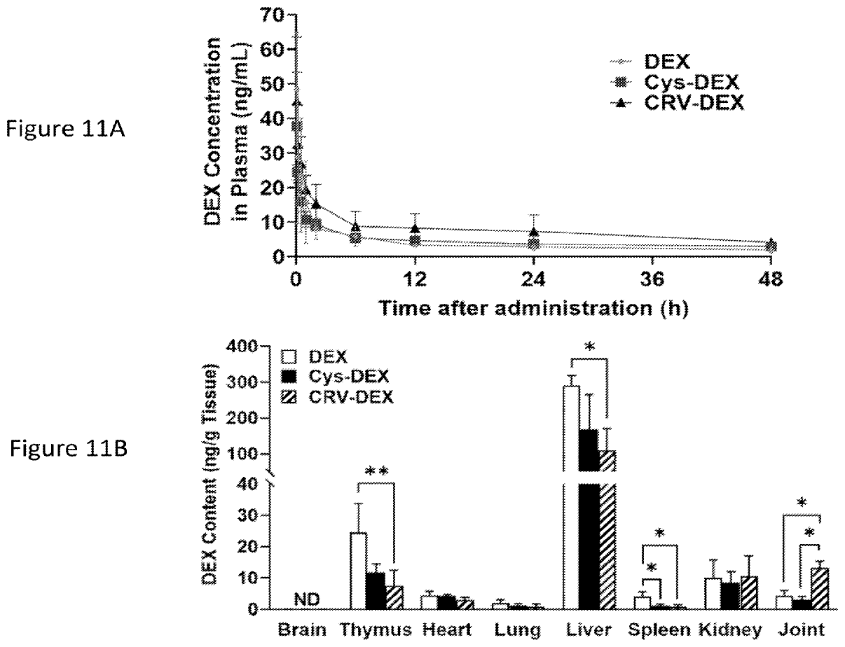

Plasma pharmacokinetics of CRV-DEX was evaluated in healthy mice. After intravenous injection of DEX, Cys-DEX and CRV-DEX at a dosage of 1 mg kg⁻¹ (equivalent DEX weight), their plasma half-life (T_{1/2}) and area under the plasma concentration curve (AUC) were analyzed using a noncompartmental model. CRV conjugates showed a prolonged T1/2 and about one-fold increase of AUC compared to DEX alone (Table 44 and FIG. 11A).

TABLE 5
Pharmacokinetics of DEX after intravenous administration
of free DEX, Cys-DEX, and CRV-DEX to mice (1
mg/ml equivalent dose of DEX per mouse)

| Pharmacokinetic | | | |
|---|---|---|---|
| Parameters | DEX | Cys-DEX | CRV-DEX*# |
| T_{1/2}(h) | 1.91 ± 0.34 | 2.77 ± 1.82 | 3.19 ± 0.56 |
| AUC₀₋ₜ(ng*h/mL) | 23.18 ± 5.14 | 29.33 ± 2.02 | 39.29 ± 9.38* |

Values are expressed as mean ± SD (n = 3),
*P < 0.05 relative to free DEX;
#P < 0.05 relative to Cys-DEX.
T_{1/2} = half-life.
AUC₀₋ₜ = area under the concentration-time curve.

Values are expressed as mean±SD (n=3), *P<0.05 relative to free DEX; #P<0.05 relative to Cys-DEX. T_{1/2}=half-life. AUC₀₋ₜ=area under the concentration-time curve.

We have shown previously that only free glucocorticoids are the active component implicating in therapeutic efficacy and side effects. Therefore, the in vivo biodistribution of CRV-DEX conjugates in CIA mice was analyzed by the quantification of the amount of DEX in various tissues of CIA mice by mass spectrometry. The tissues were collected from CIA mice after 3 h intravenous injection of DEX, Cys-DEX, and CRV-DEX at a dose of 1 mg kg⁻¹ in DEX weight, respectively. CRV-DEX group exhibited a higher amount of DEX in the joint, but a less accumulation at the liver, thymus, and spleen than DEX group (FIG. 11B). There was little difference among three groups in other organs including the lung, heart, and kidney (no detectable DEX signal in the brain) (FIG. 11B). Taken together, CRV conjugation was able to increase the DEX accumulation in the arthritic joints, while reducing it in immune organs or has no impact in healthy organs.

### CRV Conjugation Improves the Therapeutic Efficacy and Safety of Drugs Against RA

To evaluate the impact of CRV conjugation on DEX against synovial inflammation at arthritic joint, the CIA mice were treated with a low dose of DEX, Cys-DEX, or CRV-DEX (0.01 mg kg⁻¹, equivalent DEX weight) every two days for one week as schemed in FIG. 12A. While free DEX and Cys-DEX showed little effect on the severity of joint swelling, CRV-DEX blocked disease development in CIA mice (FIG. 12B). At the end of treatment, histology evaluation was performed by hematoxylin and eosin (H&E) and Safranin O staining on joint tissues, and the scores of joint damage were quantified according to the formation of pannus (synovial inflammation), proteoglycan loss, cartilage, and bone erosion. Accordingly, CRV-DEX significantly attenuated the severity of synovial inflammation and bone and cartilage destruction, showing a stronger efficacy than free DEX and Cys-DEX (FIG. 12C). In addition to the histology results, micro-computed tomography (micro-CT) was used to assess the bone damage in inflamed ankle joints of CIA mice after CRV-DEX treatment. Compared with normal mice, the CIA mice treated with free DEX and Cys-DEX had a rough and severely eroded bone surface, and a much low (bone volume/tissue volume) BV/TV ratio and bone mineral density (BMD), whereas those treated with CRV-DEX conjugation exhibited a reduced joint destruction with a smooth bone surface and yielded a BV/TV and BMD close to that of normal mice.

In a similar experiment to evaluate the impact of CRV conjugation on the ability of the anti-inflammatory drug dexamethasone (DEX) to attenuate joint inflammation, CIA mice with collagen-induced arthritis were treated intravenously with free DEX or CRV-DEX at a dosage of DEX equivalent to 0.01 mg kg⁻¹ or 0.1 mg kg⁻¹ of free DEX by weight every two days for seven days. At both dosages, CRV-DEX displayed a stronger ability than DEX to attenuate paw swelling in arthritic mice.

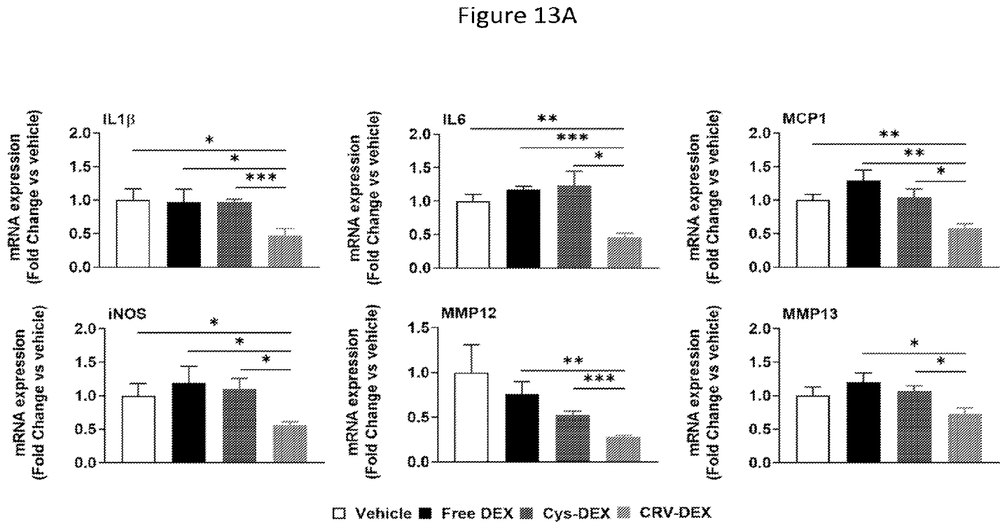

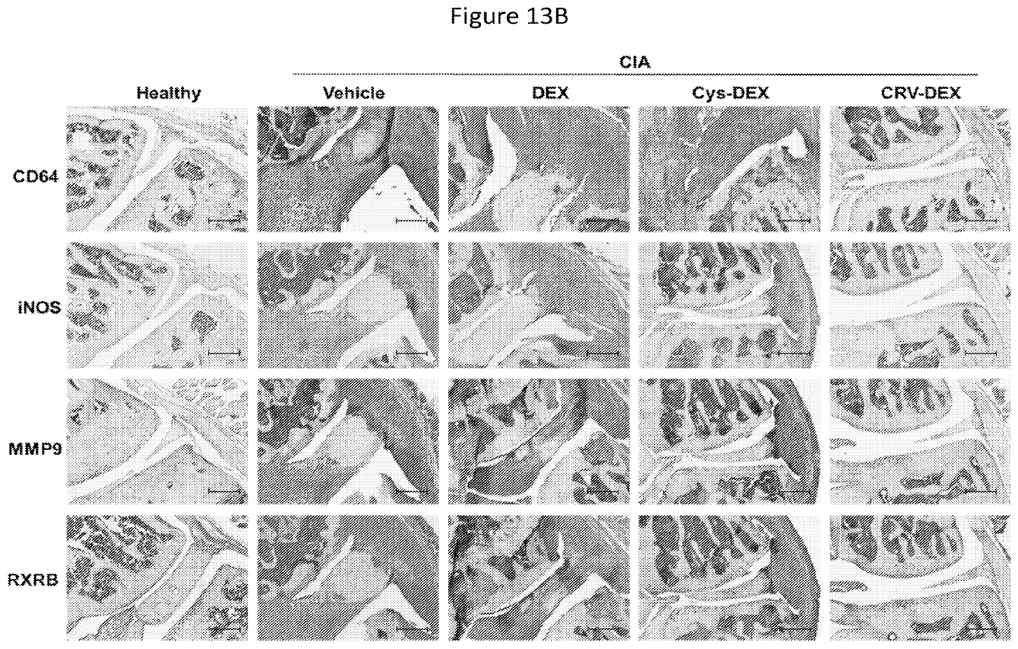

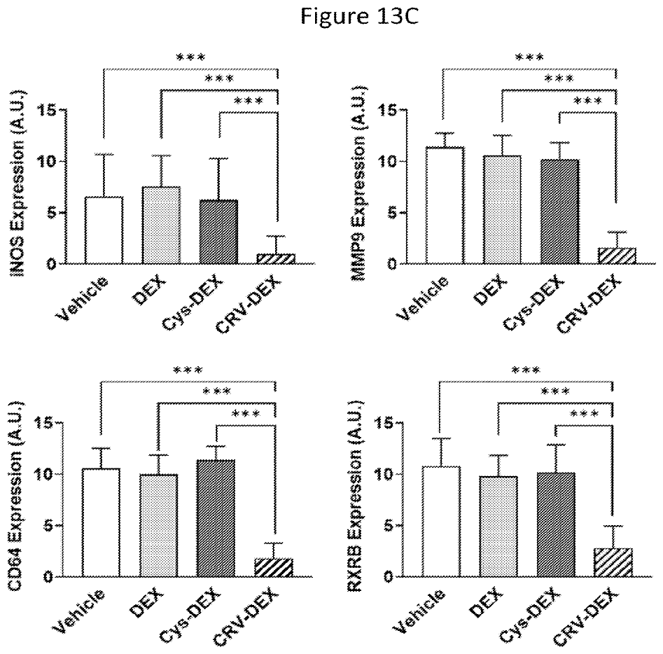

Consistently, the mRNA expression of inflammatory markers including IL1β, IL6, MCP1, iNOS, MMP12, and MMPP13 was significantly reduced in the inflamed joints of CIA mice treated with CRV-DEX, indicating the increased effective inhibition of synovial inflammation (FIG. 13A). Immunohistochemical analysis of the expression of inflammatory markers including CD64, iNOS, and MMP9 further supported the beneficial effect of CRV-DEX on the inhibition of inflammation over DEX and Cys-DEX at arthritic joints in vivo (FIG. 13B-C). These results confirm that CRV conjugation improves the DEX therapeutic efficacy with a stronger suppressive activity in inflammation and thus alleviates the arthritic injury in CIA mice.

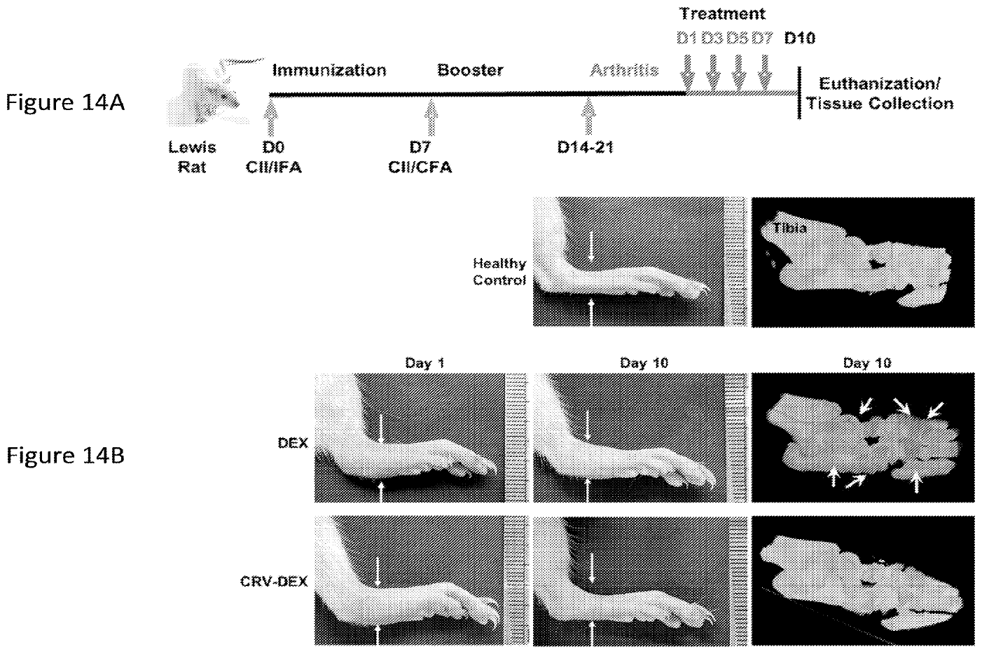

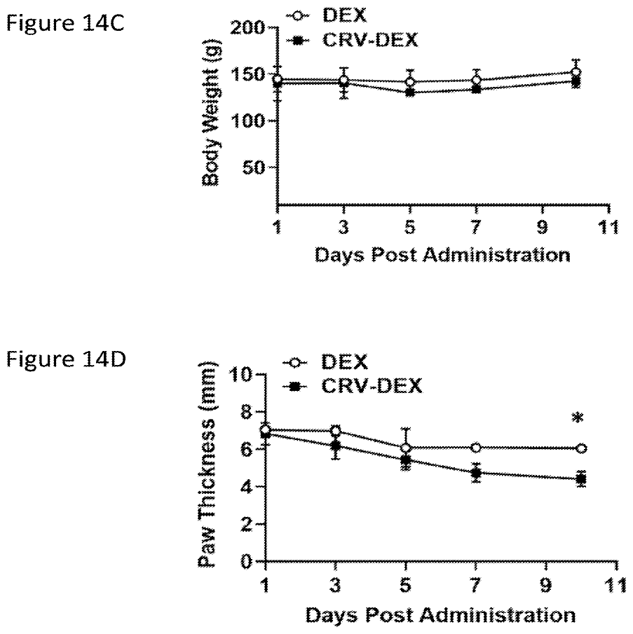

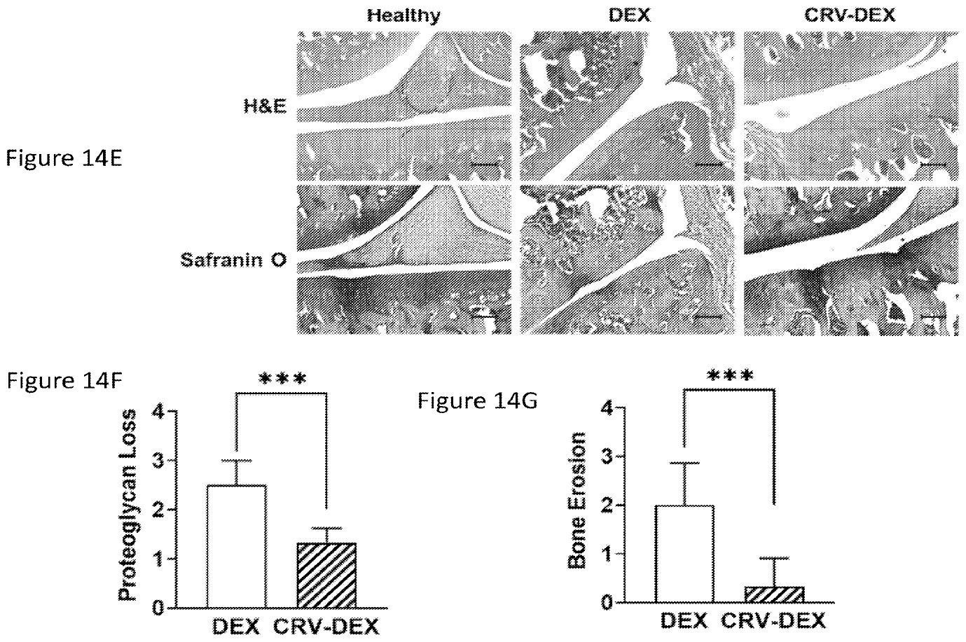

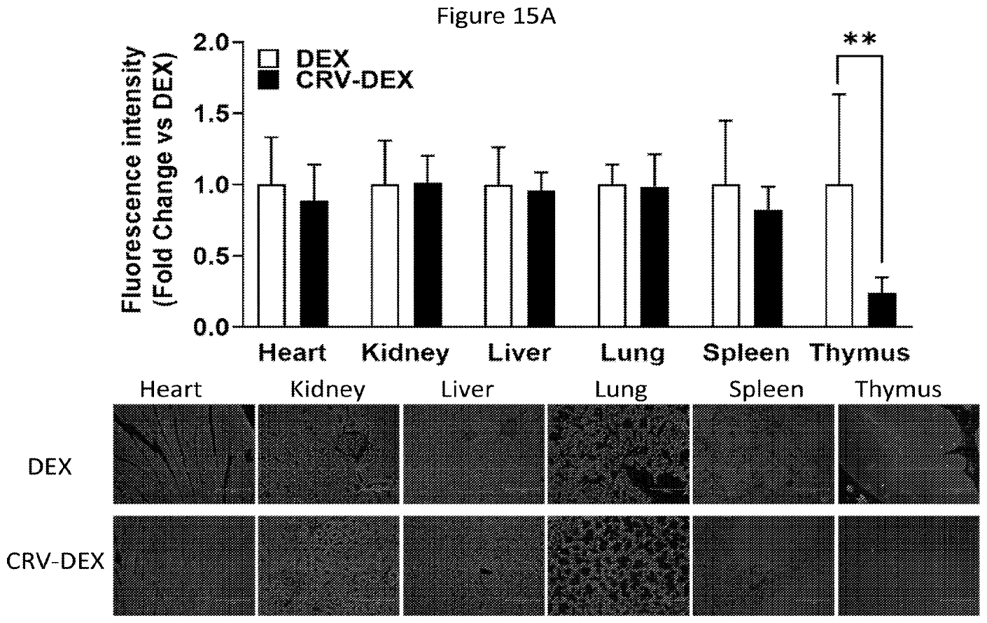

Moreover, the beneficial effect of CRV conjugation on the improvement of anti-inflammation of DEX in arthritis was performed in a CIA rat model, as evaluated by the severity of arthritis and the levels of paw swelling, compared to those treated with free DEX (FIG. 14 A-D). Like the findings in CIA mice, CRV-conjugation also resulted in an improved therapeutic efficacy as evaluated by cartilage and bone damage using H&E, Safranin O staining, and micro-CT scan of arthritic joints (FIG. 14E).

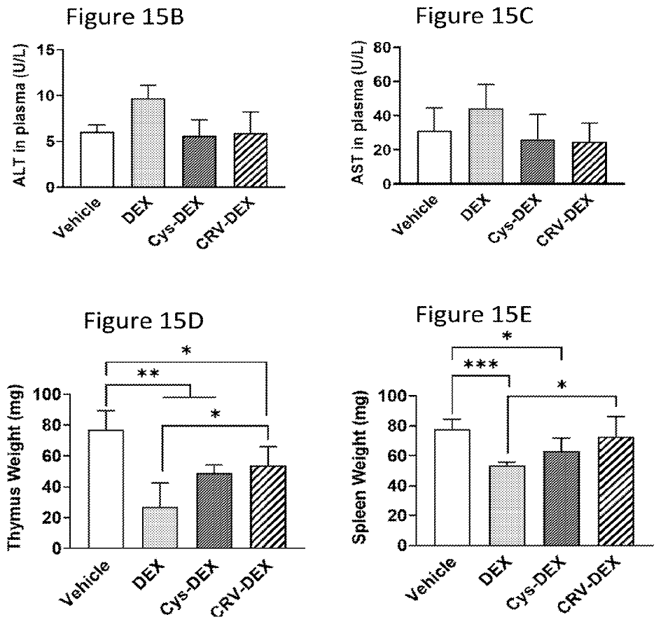

Additionally, we examined the commonly seen systemic side effects including the reduced thymus and spleen weight due to increased apoptosis after a short-term low dose and a long-term glucocorticoid usage. For the short-term side effects of DEX, the CIA mice were given intravenous injection of DEX, Cys-CRV, CRV-DEX every two days for one week with an equivalent dose of DEX at 0.01 mg kg⁻¹. In line with our previous report using CRV-conjugated PSL in mice, the TUNEL (terminal deoxynucleotidyl transferase dUTP nick end labeling) staining showed that free DEX treatment increases the level of apoptosis mainly in the thymus, which could be reduced by CRV conjugation (FIG. 15A). The levels of liver enzymes aspartate transaminase (AST) and alanine transaminase (ALT) in the blood showed no significant difference among all groups (FIG. 15B-C). Further, a long-term exposure of glucocorticoids was performed in healthy mice receiving DEX, Cys-DEX, or CRV-DEX with an equivalent dose of DEX at 2 mg kg⁻¹ via daily intravenous injection for 2 weeks. While DEX caused the most significant weight loss of thymus and spleen, Cys conjugation rescued it to some extent and CRV conjugation was able to restore it almost back to that of healthy controls (FIG. 15D-E). The livers enzymes of ALT and AST showed no significant difference among all groups after a long-term treatment. Similar to the changes of the short-term exposure of treatment, the TUNEL staining after long-term exposure of DEX displayed increased apoptosis in thymus and spleen, which could be reduced by CRV conjugation (FIG. 15A). The above findings indicate that CRV conjugation attenuates the short-term and long-term toxicity of DEX after systemic administration.

### Discussion

In this study, we investigated the impact of peptide-guided delivery in the improvement of the therapeutic efficacy and the reduction of long-term side effects of DEX in treating RA. We found that CRV peptides can selectively accumulate at the inflammatory synovial tissues of arthritic joints of CIA mice upon intravenous injection, which may be based on the elevation of RXRB expression on the surface of myeloid infiltrates. With its specific targeting ability, CRV was shown to improve the biodistribution of DEX more towards the inflamed joints of CIA mice. The covalently conjugated CRV-DEX, with a ROS-responsive linker in the middle, remained stable and intact in the plasma until entry into the cells. The intracellularly cleaved DEX from CRV conjugation in response to oxidative stress from inflammatory stimulation was able to exert its immunosuppressive activity in vitro as the free DEX. CRV conjugation also increased the accumulation of DEX in the arthritic joints in vivo so as to improve its therapeutic efficacy in the reduction of synovial inflammation, cartilage, and bone destruction. Accordingly, CRV conjugation guided the selective accumulation of DEX at the inflamed joints other than healthy organs of CIA mice so that it was able to lower the systemic side effects of DEX. Overall, we demonstrate that the peptide-guided delivery by CRV conjugation may improve the efficacy and long-term safety of glucocorticoids use in RA.

RA is a chronic, progressive, inflammatory autoimmune disease associated with synovial inflammation, which can cause joint destruction and lead to physical disability. To relieve symptomatic pain and alleviate inflammation associated with RA, the conventional drugs including non-steroidal anti-inflammatory drugs (NSAIDs), glucocorticoids, and synthetic or biologic disease-modifying anti-rheumatic drugs (DMARDs) are typically used for the treatment. However, these drugs in use are moderately efficacious on the reduction of inflammation and are ineffective in delaying disease progression. In addition, systemically administered pharmacotherapies distribute indiscriminately in healthy tissues and thus their non-specific targeting causes extraarticular side effects and even leads to life threating consequences along with impaired immune function. Hence, targeted therapies that only exert their actions at the diseased site are highly desirable so as to allow the lower dose administration and to reduce the risk of side effects.

Innate immune system including macrophages and neutrophils play an essential role in the pannus formation and a hyperplastic synovium at the onset and the development stage of RA. Orchestrated by these inflammatory cells, the accelerated levels of numerous proinflammatory cytokines, enhanced generation of ROS, and increased matrix-degrading enzymes amplify synovial inflammation and further promote cartilage damage. Since these myeloid cells are the most abundant cell types in the synovium of RA and are associated with disease initiation and progression, this makes them an ideal target for the anti-inflammation treatment of RA. Macrophages and neutrophils have been shown to serve as the target of treatment in RA, and several cell-specific targeting ligands including folate receptors-β (FR-β), CD44 receptors, mannose receptors, and other scavenger receptors have been explored to target macrophages for RA. Although these targeted delivery systems have been proven effective for specific delivery of therapeutic agents to the inflammatory site of RA, a suitable ligand that can direct the drug to the targeted tissue is still desired to improve the efficacy and safety for the treatment of RA.

Agreeing with others, we found significant infiltration of neutrophils and macrophages at the inflamed joints of CIA mouse model with reduced lymphocytes at rheumatoid synovial tissues. Our previous reports have shown that the RXRB expression is specifically elevated on the surface of myeloid cells at the inflammation site of lung tissue in acute lung injury model as well as on the tumor associated macrophages in different types of tumors. In the present study, the cell surface level of RXRB was found increased mainly in the myeloid infiltrates at the inflamed joints other than healthy ones in murine CIA model, suggesting the potential of RXRB as an inflammatory marker or ligand for targeting. Besides murine CIA model, we also validated these findings with KR×N serum-transferred arthritis mouse model, rodent CIA model, as well as with the synovial specimens from the patients with rheumatoid arthritis. In human subjects with RA, high infiltration of myeloid cells at the inflammatory joints are correlated with the RA progression and disease activity. Our results show that the upregulated gene expression of RXRB in synovial tissues from the patients with RA is accompanied with significantly increased inflammatory cytokines, and dysregulated matrix metalloproteases (MMPs), the regulators of synovial inflammation and cartilage remodeling in RA. Histology of arthritic synovial tissues from human subjects with RA further supports the evidence of elevated RXRB in active RA, suggesting the correlation of RXRB with disease activity. It is of great clinical relevance and significance to better understand of the regulatory mechanism of RXRB with immune infiltrate subsets in RA and to find a novel target for the treatment.

As the site-specific recruitment of neutrophils and macrophages from the blood contribute to the pathogenesis of rheumatoid arthritis, and these myeloid cells possess a high plasticity of gene expression in response to various stimuli, it is likely that RXRB expression may be increased from these infiltrated cells at the synovial tissues in response to inflammatory milieu at joints. Although RXRB has been regarded as one of the superfamilies of nuclear receptors (NRs) and is widely expressed at various tissues, the exact mechanisms of RXRB regulation of these immune cells and the path-physiological functions of RXRB-positive cells, remain to be further investigated. Nonetheless, being the receptor of CRV, the differentially expressed RXRB in inflammatory cells, particularly at the cell surface, may provide the basis of the targeting specificity of CRV in the inflamed joints of arthritis. Agreeing to our speculation, the whole body scanning by PET/CT after systemic administration of ⁶⁴Cu labeled CRV showed that the peptide significantly accumulates at the inflamed joints of CIA mice, which was not observed in normal mice or the peptide control in CIA mice. These are further supported by flow cytometry that the peptide is able to bind to the surface of neutrophils and macrophages isolated from inflamed joints of CIA model, which may be through the elevated RXRB on these inflammatory infiltrates in arthritic joints.

Owing to the potent and rapid anti-inflammatory effects, systemic glucocorticoids are frequently used in the management of early and moderate arthritis, or in combination with other regiments for the exacerbations of RA in clinical practice. However, systemic long-term or high dose use of glucocorticoids in the treatment of RA is accompanied by a variety of adverse consequences including immunosuppression, osteoporosis, and increased cardiovascular and infection risks. It is likely that the hydrophobic feature of these drugs attributes to the non-specific accumulation in healthy organs and poor biodistribution at the diseased sites upon systemic administration. To improve targeting specificity, several carrier systems such as nanoparticles (NPs) and polymers have been developed by passive targeting through enhanced permeability and retention (EPR) mechanism, or by active targeting via cell-specific targeting ligand into arthritic joints of RA. Nanoparticles such as liposomes encapsulating PSL or DEX for synovium targeting have been proven to improve therapeutic efficacy in murine and rodent arthritis models. The peptide-targeted liposomal delivery of DEX has further improved arthritis therapy. However, a significant amount of nanoparticles accumulates at liver and spleen by the reticuloendothelial system (RES) uptake due to their sizes and hydrophobic surfaces.

One of the most commonly used polymers such as poly(ethylene glycol) (PEG), which are highly flexible and hydrophilic, have been proven to resist uptake by the RES. This feature helps prolong the blood circulation time, improve pharmacokinetic properties of associated drugs, and widen the therapeutic window for their targeting. Drug modification with PEG has been applied to modify the liposomes for the targeted delivery of DEX in RA and showed that PEG-liposome-DEX displays an increased half-life and a better inflamed joint targeting than unmodified liposomes. PEG-DEX conjugates have also been synthesized and displayed its beneficial effect on anti-inflammation by systemic administration to arthritic rats compared to free DEX. Additionally, peptide-guided delivery of glucocorticoids has been explored to limit the off-tissue toxicity in treating rheumatoid arthritis, ocular diseases, and obesity. Hence, by adapting our previous methods, we synthesized CRV-DEX conjugation linked with PEG. Since DEX is a hydrophobic compound that can diffuse freely through cellular membrane, which causes off-target tissue distribution when given systemically. To control the release of DEX from the conjugate, different linkers including pH sensitive and thermos-responsive linkers have been developed to ensure their cleavage in a pathophysiological environment for a superior anti-inflammatory effects and biosafety. Since strong evidence suggests that a large amount of ROS is produced by infiltrated immune cells and other infiltrates at synovium in active RA, which causes oxidative stress in RA synovial microenvironment and leads to further tissue damage. Therefore, we used a ROS-responsive linker in the present study to ensure the release of DEX after entry into target cells in response to high-level ROS at inflamed joints.

Consistent with our previous findings of CRV-PSL, the ROS linker remained intact in the plasma of CIA mice and CRV-DEX exhibited an equivalent immunosuppressive activity to free DEX to inhibit inflammation in LPS-stimulated RAW 264.7 cells, which suggest the cleavage of DEX from PEG. These in vitro results indicate the potential use of CRV-DEX in vivo that the conjugate is stable in circulation until they are exposed to oxidative conditions inside inflammatory cells for cleavage and followed anti-inflammatory action by cleaved DEX. The PEGylation in the CRV-DEX conjugate used in this study offers the advantage over free DEX of a prolonged half-life and a reduced uptake by the RES, leading to enhanced accumulation at inflamed joints. This permits the achievement of the therapeutic efficacy with the systemic administration of lower doses by CRV-DEX. In comparison to using an equivalent dose of DEX from 0.1 mg kg⁻¹ to 10 mg/kg in other studies, we gave the arthritic mice an extremely low dosage (equivalent dose of DEX at 0.01 mg kg⁻¹) via intravenous injection. CRV-DEX exhibited the prominent activity to reduce synovial inflammation, and cartilage and bone damage. Although Cys-DEX showed a prolonged half-life in the blood and a reduced the accumulation in spleen, no significant therapeutic efficacy was achieved in the treatment. This further demonstrates that targeting is indeed needed for the best efficacy.

Besides the significant improvement of therapeutic efficacy, CRV-DEX conjugate successfully minimized the side effects in healthy organs, which has largely restricted the clinical applications for long-term usage of glucocorticoids in RA. Immunosuppression is one of the adverse consequences of glucocorticoids in the treatment of RA due to their non-specific organ toxicity by inducing apoptosis in thymus and spleen. We showed that less CRV-DEX accumulated at thymus and spleen than free DEX when given intravenously. Low-dose short-term treatment of DEX (0.01 mg kg⁻¹, four doses) caused the toxicity of thymus with increased apoptosis, which could be limited by CRV-DEX. The advantage of CRV-DEX is even more pronounced when given a long-term high-dose (2 mg kg⁻¹, 14 doses). Free DEX causes significant atrophy of thymus and spleen, while CRV-DEX can minimize the damage. Cys-DEX partially reduced toxicity, likely due to less accumulation at these healthy immune organs.

In conclusion, we identify a peptide ligand RXRB on myeloid infiltrate at inflamed joints of active RA. Targeting RXRB by CRV-conjugation enables an advantageous selective drug accumulation in disease sites, and a controlled intracellular drug release. CRV-DEX not only exhibits a pronounced efficacy of alleviating synovial inflammation and joint damage but also minimizes the long-term side effects of DEX. It is of great potential to apply the strategy to other drugs for RA treatment, and other diseases required for long-term glucocorticoids for a better therapeutic efficacy and safety in the treatment.

### Example 3: D-Isoform of CRV for Increased Stability

In the following example, the inventors demonstrate that the increased stability of the D-isoform of CRV allows it to be effectively administered orally.

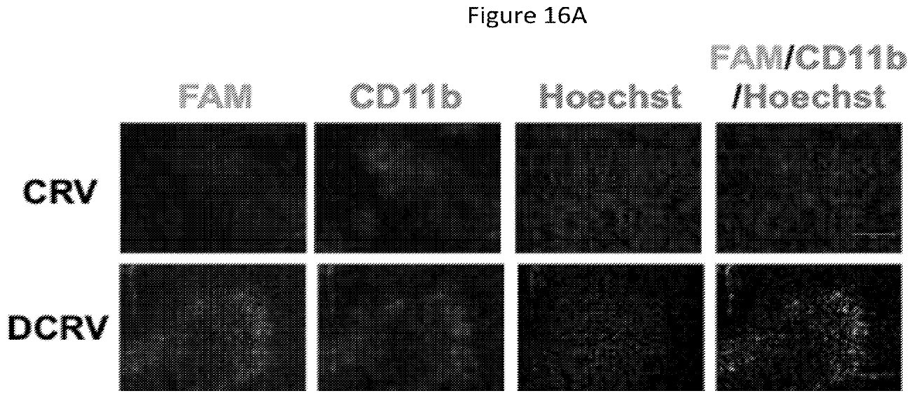

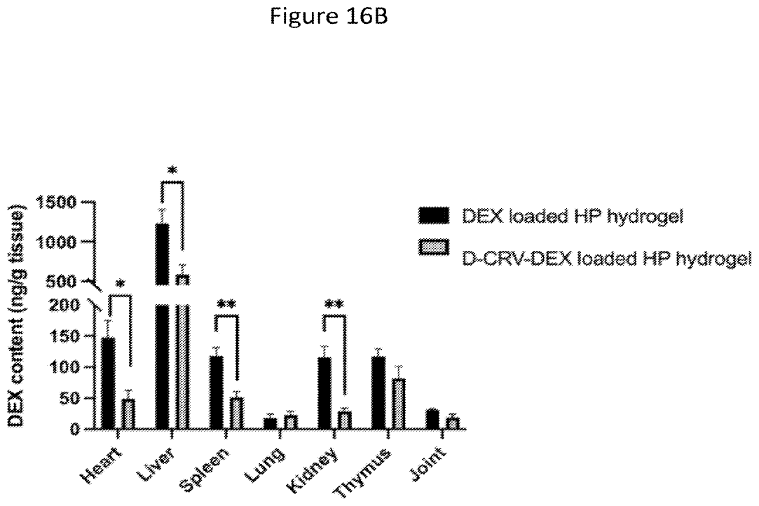

To compare the in vivo distributions of the D-isoform of CRV (D-CRV) and the L-isoform of CRV (L-CRV), fluorescein (FAM) dye labeled D-CRV and L-CRV peptides were synthesized. 100 μL of peptide solution (5 mg/mL in PBS) was administered via oral gavage into mice bearing a 4T1 breast cancer tumor (for a description of how this mouse model is generated, see J Control Release 301:42-53, 2019) 4 hours before the tumors were collected. Peptide accumulation in the tumor was observed by immunofluorescence (IF) staining of FAM. We had previously shown that L-CRV homes to such tumors following intravenous injection. However, following oral injection, we saw significant accumulation of CRV in the tumors of only the D-CRV treated mice and not the L-CRV treated mice (FIG. 16A). Further, D-CRV showed colocalization with the myeloid cell marker CD11b in the tumor (FIG. 16A). Additionally, (D)-CRV-DEX was first applied to RA mice model via subcutaneous administration with hydrogel. The biodistribution of DEX was shown in FIG. 16B indicating that (D)-CRV-DEX successfully got into the blood vessel and was transferred into different organs after the subcutaneous administration. The pharmacokinetics (PK) and biodistribution at other time points will be further evaluated. These data suggest that D-CRV is functional and successfully homes to tumor immune cells upon oral or subcutaneous administration.

### Example 4: Changing the Size or Type of Linker in DEX-PEG Conjugates

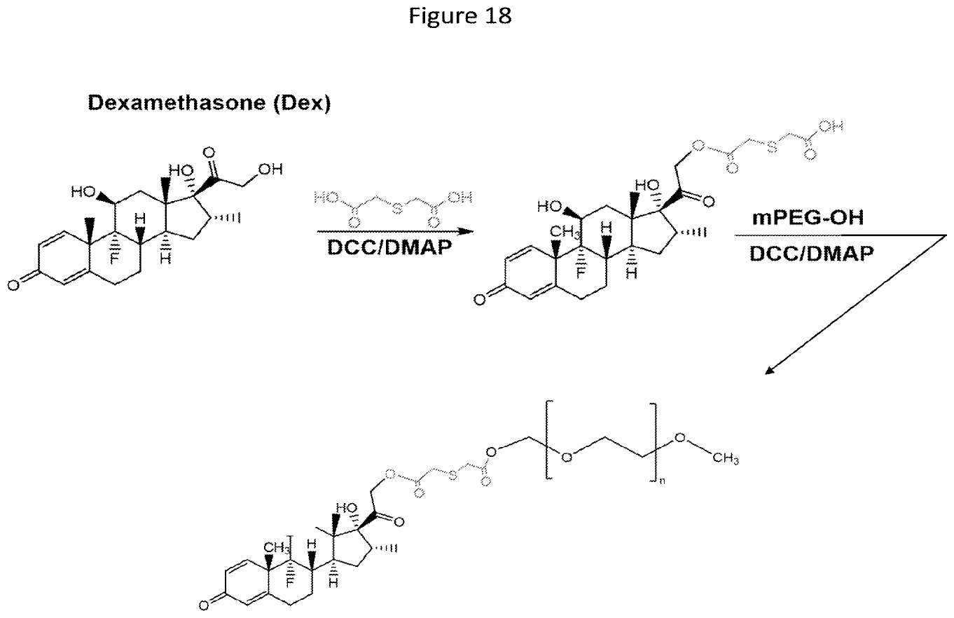

In conjugation chemistry, the PEG linker is a crucial piece. We tested the impact of polyethylene glycol (PEG) molecular weight (MW) on the homing and therapeutic outcomes of drug-conjugations. For CRV-based compounds, we have made three CRV-Dexamethasone (DEX) conjugates with varying PEG lengths (2k MW, 5k MW, and 10k MW of PEG), which are currently being evaluated for homing specificity in acute lung injury (ALI) and rheumatoid arthritis (RA) mouse models. Moreover, we recently found that fpeg200 our conjugation method (PEG plus ROS-responsive linker), without CRV, can significantly reduce the adverse effect of DEX in obese mice. DEX is well known to induce fatty liver and elevated blood Triglyceride in obese patients. We found that PEG-ROS-DEX significantly reduces such adverse effects. The preparation of the conjugates is illustrated in FIG. 18.

### Results

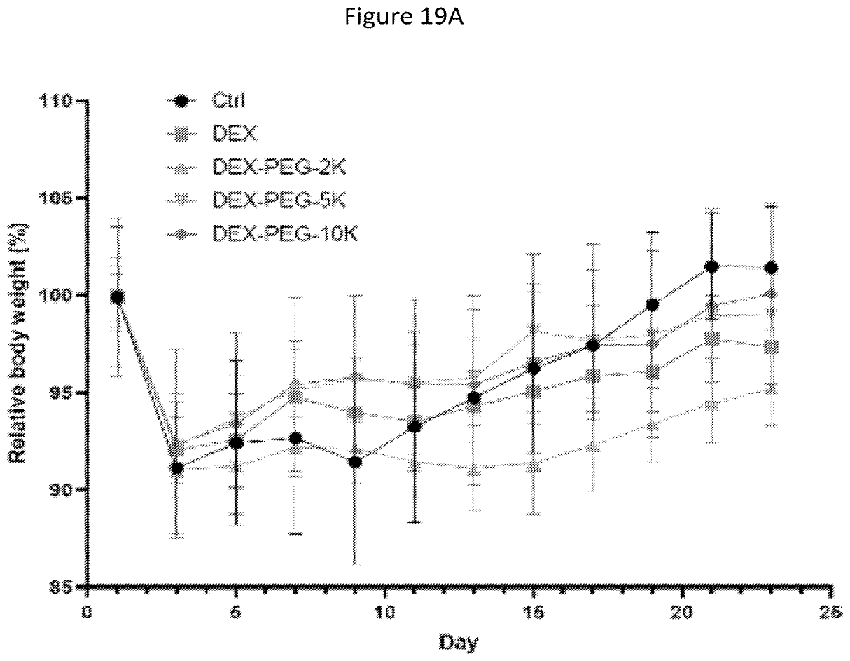

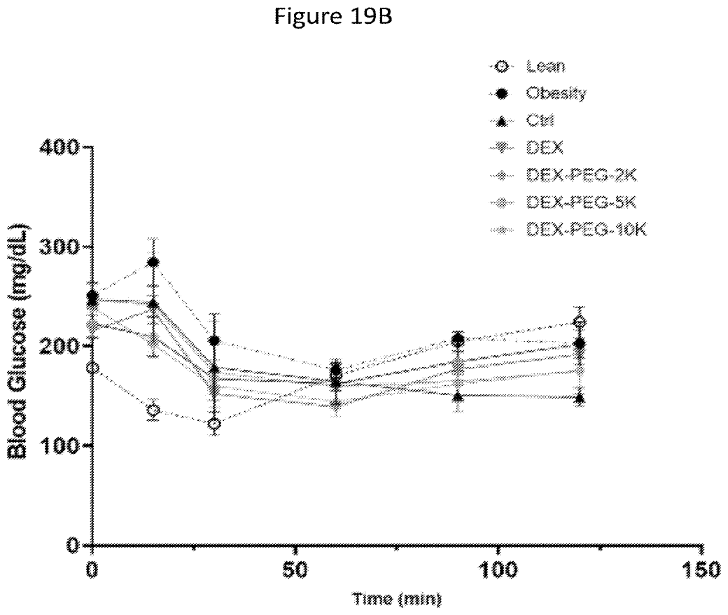

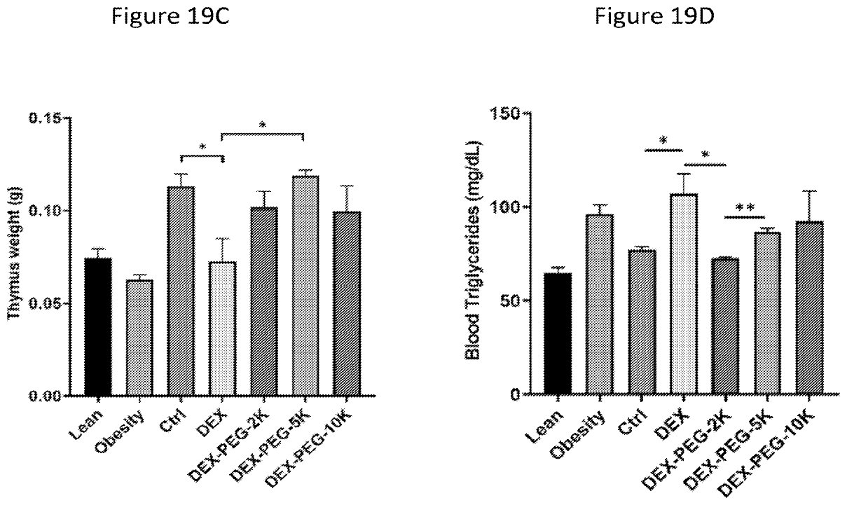

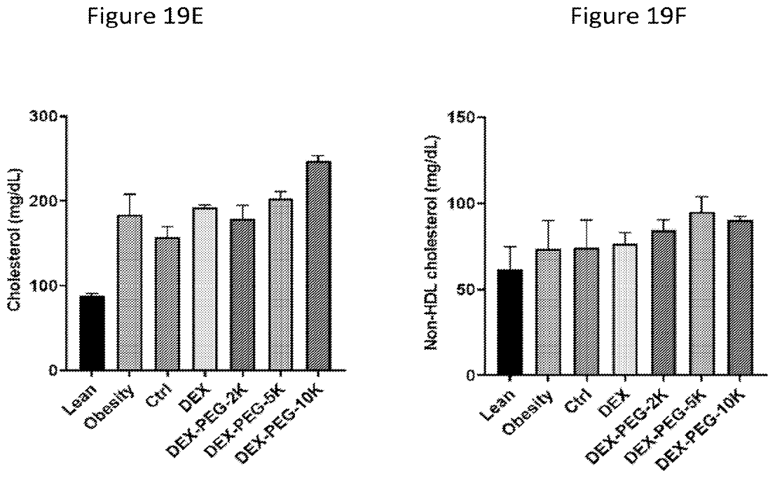

FIGS. 19A-F indicates that there were no significant differences in body weight, insulin tolerance, and plasma cholesterol among the groups. All three DEX-conjugations were able to restore the thymus weight of the mice to that of the control group. Notably, DEX-PEG5K showed a significant difference in this regard. Additionally, DEX-PEG2k significantly decreased the level of triglycerides in the plasma as compared to the other groups, as shown in FIG. 19D. This result was consistent with the findings from the H&E analysis depicted in FIG. 3.

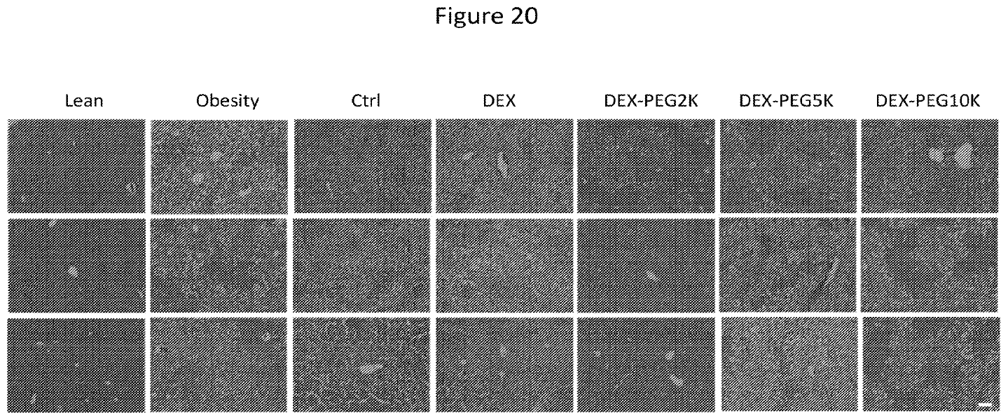

DEX or DEX-PEG induces no change on body weights or blood glucose (FIG. 19A-B). DEX reduces thymus weight, a well-known adverse effect, while PEG conjugation recovers it (FIG. 19C). DEX elevates the blood triglyceride (Ctrl vs DEX), while DEX-PEG (2K) lowers it back to lean mice level (FIG. 19D). PEG does not have effect on other blood parameters (FIG. 19E-F). PEG 2K conjugation reduces the fatty liver induced by DEX (FIG. 20). In particular, large empty vacuoles are shown in the fatty liver.

Drug-Conjugation with Other Cleavable Linkers

In addition to the ROS-sensitive cleavable linker, we also prepared a pH-sensitive linker to investigate whether other types of linkers could be used in conjunction with our prodrug strategy. FIG. 21A shows the route of the pH-sensitive conjugation synthesis. FIG. 21B demonstrates the in vitro release of DEX from the conjugation under pH 5 and pH 7 conditions, respectively. In vivo drug release will be carried out in the future.

### Experimental Procedure

Male C₅₇ mice were subjected to high-fat diet feeding for 8 weeks to induce obesity. An acute lung injury (ALI) model was established in the obesity mice by administering 30 μg of LPS intratracheally per mouse. Four days following LPS administration, the mice were randomly allocated into 5 groups (n=4):
1) Control group: received 100 μL of PBS via intravenous (i.v.) injection through the tail vein every other day for 3 weeks.
2) DEX group: received 100 μL of DEX solution in PBS (0.1 mg/kg of DEX) via i.v. injection through the tail vein every other day for 3 weeks.
3) DEX-PEG(2K) group: received 100 L of DEX-PEG(2k) solution in PBS (equivalent to 0.1 mg/kg of DEX) via i.v. injection through the tail vein every other day for 3 weeks.
4) DEX-PEG(5K) group: received 100 μL of DEX-PEG(5k) solution in PBS (equivalent to 0.1 mg/kg of DEX) via i.v. injection through the tail vein every other day for 3 weeks.
5) DEX-PEG(1OK) group: received 100 L of DEX-PEG(10k) solution in PBS (equivalent to 0.1 mg/kg of DEX) via i.v. injection through the tail vein every other day for 3 weeks.

In addition, lean mice and obese mice without ALI were included in the study as controls and received no treatment. Body weight of the mice was monitored every other day during the study. One day prior to sacrificing the mice, an insulin tolerance test (ITT) was conducted. Blood and major organs were collected at the end of the study, and plasma triglycerides, total and non-HDL cholesterol were quantified. The organs were weighed and sectioned for H&E staining.

### REFERENCES

Matthay M A, Zemans R L. The acute respiratory distress syndrome: pathogenesis and treatment. Annu Rev Pathol 2011; 6: 147-163.
Confalonieri M, Salton F, Fabiano F. Acute respiratory distress syndrome. European Respiratory Review 2017; 26: 160116.
Group R C, Horby P, Lim W S, Emberson J R, Mafham M, Bell J L, Linsell L, Staplin N, Brightling C, Ustianowski A, Elmahi E, Prudon B, Green C, Felton T, Chadwick D, Rege K, Fegan C, Chappell L C, Faust S N, Jaki T, Jeffery K, Montgomery A, Rowan K, Juszczak E, Baillie J K, Haynes R, Landray M J. Dexamethasone in Hospitalized Patients with Covid-19. N Engl J Med 2021; 384: 693-704.
Liu J, Zhang S, Dong X, Li Z, Xu Q, Feng H, Cai J, Huang S, Guo J, Zhang L, Chen Y, Zhu W, Du H, Liu Y, Wang T, Chen L, Wen Z, Annane D, Qu J, Chen D. Corticosteroid treatment in severe COVID-19 patients with acute respiratory distress syndrome. The Journal of Clinical Investigation 2020; 130: 6417-6428.
Steinberg K P, Hudson L D, Goodman R B, Hough C L, Lanken P N, Hyzy R, Thompson B T, Ancukiewicz M. Efficacy and safety of corticosteroids for persistent acute respiratory distress syndrome. N Engl J Med 2006; 354: 1671-1684.
Peter J V, John P, Graham P L, Moran J L, George I A, Bersten A. Corticosteroids in the prevention and treatment of acute respiratory distress syndrome (ARDS) in adults: meta-analysis. Bmj 2008; 336: 1006-1009.
Kim B, Pang H B, Kang J, Park J-H, Ruoslahti E, Sailor M J. Immunogene therapy with fusogenic nanoparticles modulates macrophage response to Staphylococcus aureus. Nature Communications 2018; 9: 1969.
Tang T, Wei Y, Kang J, She Z G, Kim D, Sailor M J, Ruoslahti E, Pang H B. Tumor-specific macrophage targeting through recognition of retinoid X receptor beta. Journal of controlled release: official journal of the Controlled Release Society 2019; 301: 42-53.
Frank J A, Wray C M, McAuley D F, Schwendener R, Matthay M A. Alveolar macrophages contribute to alveolar barrier dysfunction in ventilator-induced lung injury. Am J Physiol Lung Cell Mol Physiol 2006; 291: L1191-1198.
Johnston L K, Rims C R, Gill S E, McGuire J K, Manicone A M. Pulmonary macrophage subpopulations in the induction and resolution of acute lung injury. Am J Respir Cell Mol Biol 2012; 47: 417-426.
Vandevyver S, Dejager L, Libert C. Comprehensive overview of the structure and regulation of the glucocorticoid receptor. Endocr Rev 2014; 35: 671-693.
Liang J, Liu B. ROS-responsive drug delivery systems. Bioengineering & Translational Medicine 2016; 1: 239-251.
Saravanakumar G, Kim J, Kim W J. Reactive-Oxygen-Species-Responsive Drug Delivery Systems: Promises and Challenges. Advanced Science 2017; 4: 1600124.
Matute-Bello G, Frevert C W, Martin T R. Animal models of acute lung injury. Am J Physiol Lung Cell Mol Physiol 2008; 295: L379-399.
Thepen Ta. Resolution of cutaneous inflammation after local elimination of macrophages. Nature Biotechnology 2000; 18: 48-51, pmid=10625390.
Xini A, Pistiki A, Lada M, Giamarellos-Bourboulis E J, Dimopoulos G. Association of the early absolute CD64-expressing neutrophil count and sepsis outcome. Eur J Clin Microbiol Infect Dis 2019; 38: 1123-1128.
Yu Y R, O'Koren E G, Hotten D F, Kan M J, Kopin D, Nelson E R, Que L, Gunn M D. A Protocol for the Comprehensive Flow Cytometric Analysis of Immune Cells in Normal and Inflamed Murine Non-Lymphoid Tissues. PLoS One 2016; 11: e0150606.
Zhu J, Feng B, Xu Y, Chen W, Sheng X, Feng X, Shi X, Liu J, Pan Q, Yu J, Li L, Cao H. Mesenchymal stem cells alleviate LPS-induced acute lung injury by inhibiting the proinflammatory function of Ly6C(+) CD8(+) T cells. Cell Death Dis 2020; 11: 829-829.
Chow C W, Herrera Abreu M T, Suzuki T, Downey G P. Oxidative stress and acute lung injury. Am J Respir Cell Mol Biol 2003; 29: 427-431.
Lee S H, Gupta M K, Bang J B, Bae H, Sung H J. Current progress in Reactive Oxygen Species (ROS)-Responsive materials for biomedical applications. Adv Healthc Mater 2013; 2: 908-915.
Funk D, Schrenk H H, Frei E. Development of a novel polyethylene glycol-corticosteroid-conjugate with an acid-cleavable linker. Journal of drug targeting 2011; 19: 434-445.
Veronese F M, Schiavon O, Pasut G, Mendichi R, Andersson L, Tsirk A, Ford J, Wu G, Kneller S, Davies J, Duncan R. PEG-doxorubicin conjugates: influence of polymer structure on drug release, in vitro cytotoxicity, biodistribution, and antitumor activity. Bioconjugate chemistry 2005; 16: 775-784.
Keller J N, Hanni K B, Gabbita S P, Friebe V, Mattson M P, Kindy M S. Oxidized lipoproteins increase reactive oxygen species formation in microglia and astrocyte cell lines. Brain Res 1999; 830: 10-15.
Lee J A, Song H Y, Ju S M, Lee S J, Kwon H J, Eum W S, Jang S H, Choi S Y, Park J S. Differential regulation of inducible nitric oxide synthase and cyclooxygenase-2 expression by superoxide dismutase in lipopolysaccharide stimulated RAW 264.7 cells. Exp Mol Med 2009; 41: 629-637.
Barish G D, Downes M, Alaynick W A, Yu R T, Ocampo C B, Bookout A L, Mangelsdorf D J, Evans R M. A Nuclear Receptor Atlas: macrophage activation. Mol Endocrinol 2005; 19: 2466-2477.
Horton D L, Remick D G. Delayed addition of glucocorticoids selectively suppresses cytokine production in stimulated human whole blood. Clin Vaccine Immunol 2010; 17: 979-985.
Ahi S, Beotra A, Dubey S, Upadhyay A, Jain S. Simultaneous identification of prednisolone and its ten metabolites in human urine by high performance liquid chromatography-tandem mass spectrometry. Drug testing and analysis 2012; 4: 460-467.
Matute-Bello G, Winn R K, Jonas M, Chi E Y, Martin T R, Liles W C. Fas (CD95) induces alveolar epithelial cell apoptosis in vivo: implications for acute pulmonary inflammation. Am J Pathol 2001; 158: 153-161.
Arkovitz M S, Wispé J R, Garcia V F, Szabó C. Selective inhibition of the inducible isoform of nitric oxide synthase prevents pulmonary transvascular flux during acute endotoxemia. J Pediatr Surg 1996; 31: 1009-1015.
Kristof A S, Goldberg P, Laubach V, Hussain S N. Role of inducible nitric oxide synthase in endotoxin-induced acute lung injury. Am J Respir Crit Care Med 1998; 158: 1883-1889.
Matute-Bello G, Downey G, Moore B B, Groshong S D, Matthay M A, Slutsky A S, Kuebler W M, Acute Lung Injury in Animals Study G. An official American Thoracic Society workshop report: features and measurements of experimental acute lung injury in animals. Am J Respir Cell Mol Biol; 2011. p. 725-738.
Brewer J A, Kanagawa O, Sleckman B P, Muglia U. Thymocyte Apoptosis Induced by T Cell Activation Is Mediated by Glucocorticoids In Vivo. The Journal of Immunology 2002; 169: 1837.
Compton M M, Cidlowski J A. Thymocyte apoptosis: a model of programmed cell death. Trends Endocrinol Metab 1992; 3: 17-23.
Cook Sangar M L, Girard E J, Hopping G, Yin C, Pakiam F, Brusniak M Y, Nguyen E, Ruff R, Gewe M M, Byrnes-Blake K, Nairn N W, Miller D M, Mehlin C, Strand A D, Mhyre A J, Correnti C E, Strong R K, Simon J A, Olson J M. A potent peptide-steroid conjugate accumulates in cartilage and reverses arthritis without evidence of systemic corticosteroid exposure. Sci Transl Med 2020; 12.
Lanza L, Scudeletti M, Puppo F, Bosco O, Peirano L, Filaci G, Fecarotta E, Vidali G, Indiveri F. Prednisone increases apoptosis in in vitro activated human peripheral blood T lymphocytes. Clin Exp Immunol 1996; 103: 482-490.
Yang J W, Mao B, Tao R J, Fan L C, Lu H W, Ge B X, Xu J F. Corticosteroids alleviate lipopolysaccharide-induced inflammation and lung injury via inhibiting NLRP3-inflammasome activation. Journal of Cellular and Molecular Medicine 2020; 24: 12716-12725.
Herold S, Mayer K, Lohmeyer J. Acute lung injury: how macrophages orchestrate resolution of inflammation and tissue repair. Front Immunol 2011; 2: 65.
Kosyreva A, Dzhalilova D, Lokhonina A, Vishnyakova P, Fatkhudinov T. The Role of Macrophages in the Pathogenesis of SARS-CoV-2-Associated Acute Respiratory Distress Syndrome. Front Immunol 2021; 12: 682871.
Lomas-Neira J, Chung C S, Perl M, Gregory S, Biffl W, Ayala A. Role of alveolar macrophage and migrating neutrophils in hemorrhage-induced priming for ALI subsequent to septic challenge. Am J Physiol Lung Cell Mol Physiol 2006; 290: L51-58.
Savov J D, Gavett S H, Brass D M, Costa D L, Schwartz D A. Neutrophils play a critical role in development of LPS-induced airway disease. Am J Physiol Lung Cell Mol Physiol 2002; 283: L952-962.
Segel G B, Halterman M W, Lichtman M A. The paradox of the neutrophil's role in tissue injury. J Leukoc Biol 2011; 89: 359-372.
Fridlender Z G, Sun J, Kim S, Kapoor V, Cheng G, Ling L, Worthen G S, Albelda S M. Polarization of tumor-associated neutrophil phenotype by TGF-beta: “N1” versus “N2” TAN. Cancer cell 2009; 16: 183-194.
Ohms M, Möller S, Laskay T. An Attempt to Polarize Human Neutrophils Toward N1 and N2 Phenotypes in vitro. Frontiers in Immunology 2020; 11.
Faverio P, De Giacomi F, Sardella L, Fiorentino G, Carone M, Salerno F, Ora J, Rogliani P, Pellegrino G, Sferrazza Papa G F, Bini F, Bodini B D, Messinesi G, Pesci A, Esquinas A. Management of acute respiratory failure in interstitial lung diseases: overview and clinical insights. BMC Pulmonary Medicine 2018; 18: 70.
Boomars K A, Wagenaar S S, Mulder P G, van Velzen-Blad H, van den Bosch J M. Relationship between cells obtained by bronchoalveolar lavage and survival in idiopathic pulmonary fibrosis. Thorax 1995; 50: 1087.
Cailes J B, Connor C, Pantelidis P, Southcott A M, Fitzgerald M X, Black C M, du Bois R M. Neutrophil activation in fibrosing alveolitis: a comparison of lone cryptogenic fibrosing alveolitis and systemic sclerosis. European Respiratory Journal 1996; 9: 992.
Bagnato G, Harari S. Cellular interactions in the pathogenesis of interstitial lung diseases. European Respiratory Review 2015; 24: 102.
Brunetti M, Martelli N, Colasante A, Piantelli M, Musiani P, Aiello F B. Spontaneous and glucocorticoid-induced apoptosis in human mature T lymphocytes. Blood 1995; 86: 4199-4205.
Buckley L, Guyatt G, Fink H A, Cannon M, Grossman J, Hansen K E, Humphrey M B, Lane N E, Magrey M, Miller M, Morrison L, Rao M, Robinson A B, Saha S, Wolver S, Bannuru R R, Vaysbrot E, Osani M, Turgunbaev M, Miller A S, McAlindon T. 2017 American College of Rheumatology Guideline for the Prevention and Treatment of Glucocorticoid-Induced Osteoporosis. Arthritis Rheumatol 2017; 69: 1521-1537.
Schäcke H, Docke W D, Asadullah K. Mechanisms involved in the side effects of glucocorticoids. Pharmacol Ther 2002; 96: 23-43.
Lühder F, Reichardt H M. Novel Drug Delivery Systems Tailored for Improved Administration of Glucocorticoids. International journal of molecular sciences 2017; 18: 1836.
Cao X T, Kim Y H, Park J M, Lim K T. One-pot syntheses of dual-responsive core cross-linked polymeric micelles and covalently entrapped drug by click chemistry. European Polymer Journal 2016; 78: 264-273.
Acharya S, Guru B. Prednisolone encapsulated PLGA nanoparticles: Characterization, cytotoxicity, and anti-inflammatory activity on C6 glial cells. Journal of Applied Pharmaceutical Science 2020; 10.
Colino C I, Lanao J M, Gutierrez-Millan C. Targeting of Hepatic Macrophages by Therapeutic Nanoparticles. Frontiers in immunology 2020; 11: 218-218.
Bhattacharya M, Sadeghi A, Sarkhel S, Hagstrom M, Bahrpeyma S, Toropainen E, Auriola S, Urtti A. Release of functional dexamethasone by intracellular enzymes: A modular peptide-based strategy for ocular drug delivery. Journal of Controlled Release 2020; 327: 584-594.
Quarta C, Clemmensen C, Zhu Z, Yang B, Joseph S S, Lutter D, Yi C X, Graf E, Garcia-Ciceres C, Legutko B, Fischer K, Brommage R, Zizzari P, Franklin B S, Krueger M, Koch M, Vettorazzi S, Li P, Hofmann S M, Bakhti M, Bastidas-Ponce A, Lickert H, Strom™, Gailus-Durner V, Bechmann I, Perez-Tilve D, Tuckermann J, Hrabě de Angelis M, Sandoval D, Cota D, Latz E, Seeley R J, Müller TD, DiMarchi R D, Finan B, Tschöp MH. Molecular Integration of Incretin and Glucocorticoid Action Reverses Immunometabolic Dysfunction and Obesity. Cell metabolism 2017; 26: 620-632.e626.
Harris J M, Chess R B. Effect of pegylation on pharmaceuticals. Nature reviews Drug discovery 2003; 2: 214-221.
Alas M, Saghaeidehkordi A, Kaur K. Peptide-Drug Conjugates with Different Linkers for Cancer Therapy. J Med Chem 2021; 64: 216-232.
Kaspar A A, Reichert J M. Future directions for peptide therapeutics development. Drug Discov Today 2013; 18: 807-817.
Scott, D.L., F. Wolfe, and T.W.J. Huizinga, Rheumatoid arthritis. The Lancet, 2010. 376(9746): p. 1094-1108.
Weyand, C.M. and J.J. Goronzy, Immunometabolism in early and late stages of rheumatoid arthritis. Nat Rev Rheumatol, 2017. 13(5): p. 291-301.
Macfarlane, E., M.J. Seibel, and H. Zhou, Arthritis and the role of endogenous glucocorticoids. Bone Research, 2020. 8(1): p. 33.
LoCascio, V., et al., Bone loss in response to long-term glucocorticoid therapy. Bone Miner, 1990. 8(1): p. 39-51.
Cooper, C., C. Coupland, and M. Mitchell, Rheumatoid arthritis, corticosteroid therapy and hip fracture. Ann Rheum Dis, 1995. 54(1): p. 49-52.
Kim, B., et al., Immunogene therapy with fusogenic nanoparticles modulates macrophage response to Staphylococcus aureus. Nature Communications, 2018. 9(1): p. 1969.
Tang, T., et al., Tumor-specific macrophage targeting through recognition of retinoid X receptor beta. J Control Release, 2019. 301: p. 42-53.
Guo, H., et al., Peptide-guided delivery improves the therapeutic efficacy and safety of glucocorticoid drugs for treating acute lung injury. Mol Ther, 2023.
Kurowska-Stolarska, M. and S. Alivernini, Synovial tissue macrophages in joint homeostasis, rheumatoid arthritis and disease remission. Nature Reviews Rheumatology, 2022. 18(7): p. 384-397.
Alivernini, S., et al., Distinct synovial tissue macrophage subsets regulate inflammation and remission in rheumatoid arthritis. Nature Medicine, 2020. 26(8): p. 1295-1306.
Zhang, L., et al., Contribution of neutrophils in the pathogenesis of rheumatoid arthritis. J Biomed Res, 2019. 34(2): p. 86-93.
Wright, H.L., R.J. Moots, and S.W. Edwards, The multifactorial role of neutrophils in rheumatoid arthritis. Nature Reviews Rheumatology, 2014. 10(10): p. 593-601.
Liang, J. and B. Liu, ROS-responsive drug delivery systems. Bioengineering & Translational Medicine, 2016. 1(3): p. 239-251.
Saravanakumar, G., J. Kim, and W.J. Kim, Reactive-Oxygen-Species-Responsive Drug Delivery Systems: Promises and Challenges. Advanced Science, 2017. 4(1): p. 1600124.
Thornton, S. and R.T. Strait, Head-to-head comparison of protocol modifications for the generation of collagen-induced arthritis in a specific-pathogen free facility using DBA/1 mice. Biotechniques, 2016. 60(3): p. 119-28.
Skovsbo Clausen, A., et al., (64)Cu-DOTATATE Positron Emission Tomography (PET) of Borrelia Burgdorferi Infection: In Vivo Imaging of Macrophages in Experimental Model of Lyme Arthritis. Diagnostics (Basel), 2020. 10(10).
Tang, T., et al., Rapid chelator-free radiolabeling of quantum dots for in vivo imaging. Nanoscale, 2019. 11(46): p. 22248-22254.
Christensen, A. D., et al., K/B×N Serum-Transfer Arthritis as a Model for Human Inflammatory Arthritis. Front Immunol, 2016. 7: p. 213.
Li, J., et al., BAD inactivation exacerbates rheumatoid arthritis pathology by promoting survival of sublining macrophages. eLife, 2020. 9: p. e56309.
Wang, J. X., et al., Ly6G ligation blocks recruitment of neutrophils via a p2-integrin-dependent mechanism. Blood, 2012. 120(7): p. 1489-98.
Huang, Q. Q., et al., Association of Increased F4/80(high) Macrophages With Suppression of Serum-Transfer Arthritis in Mice With Reduced FLIP in Myeloid Cells. Arthritis Rheumatol, 2017. 69(9): p. 1762-1771.
Tanaka, D., et al., Essential role of neutrophils in anti-type II collagen antibody and lipopolysaccharide-induced arthritis. Immunology, 2006. 119(2): p. 195-202.
Smallwood, M. J., et al., Oxidative stress in autoimmune rheumatic diseases. Free Radic Biol Med, 2018. 125: p. 3-14.
Funk, D., H.H. Schrenk, and E. Frei, Development of a novel polyethylene glycol-corticosteroid-conjugate with an acid-cleavable linker. J Drug Target, 2011. 19(6): p. 434-45.
Veronese, F. M., et al., PEG-doxorubicin conjugates: influence of polymer structure on drug release, in vitro cytotoxicity, biodistribution, and antitumor activity. Bioconjug Chem, 2005. 16(4): p. 775-84.
Keller, J. N., et al., Oxidized lipoproteins increase reactive oxygen species formation in microglia and astrocyte cell lines. Brain Res, 1999. 830(1): p. 10-5.
Lee, J. A., et al., Differential regulation of inducible nitric oxide synthase and cyclooxygenase-2 expression by superoxide dismutase in lipopolysaccharide stimulated RAW 264.7 cells. Exp Mol Med, 2009. 41(9): p. 629-37.
Hayer, S., et al., ‘SMASH’ recommendations for standardised microscopic arthritis scoring of histological sections from inflammatory arthritis animal models. Annals of the Rheumatic Diseases, 2021. 80(6): p. 714.
Griffiths, M. M., et al., Collagen-induced arthritis in rats. Methods Mol Med, 2007. 136: p. 201-14.
Brewer, J. A., et al., Thymocyte Apoptosis Induced by T Cell Activation Is Mediated by Glucocorticoids In Vivo. The Journal of Immunology, 2002. 169(4): p. 1837.
Compton, M.M. and J.A. Cidlowski, Thymocyte apoptosis: a model of programmed cell death. Trends Endocrinol Metab, 1992. 3(1): p. 17-23.
Cook Sangar, M. L., et al., A potent peptide-steroid conjugate accumulates in cartilage and reverses arthritis without evidence of systemic corticosteroid exposure. Sci Transl Med, 2020. 12(533).
Lanza, L., et al., Prednisone increases apoptosis in in vitro activated human peripheral blood T lymphocytes. Clin Exp Immunol, 1996. 103(3): p. 482-90.
Patel, R., et al., LXRβ is required for glucocorticoid-induced hyperglycemia and hepatosteatosis in mice. J Clin Invest, 2011. 121(1): p. 431-41.
Smolen, J. S., et al., Rheumatoid arthritis. Nature Reviews Disease Primers, 2018. 4(1): p. 18001.
Burmester, G.R. and J. E. Pope, Novel treatment strategies in rheumatoid arthritis. Lancet, 2017. 389(10086): p. 2338-2348.
Smolen, J. S. and G. Steiner, Therapeutic strategies for rheumatoid arthritis. Nat Rev Drug Discov, 2003. 2(6): p. 473-88.
Luís, M., et al., An updated review of glucocorticoid-related adverse events in patients with rheumatoid arthritis. Expert Opinion on Drug Safety, 2019. 18(7): p. 581-590.
Edilova, M. I., A. Akram, and A. A. Abdul-Sater, Innate immunity drives pathogenesis of rheumatoid arthritis. Biomed J, 2021. 44(2): p. 172-182.
Wright, H. L., et al., Neutrophil function in inflammation and inflammatory diseases. Rheumatology, 2010. 49(9): p. 1618-1631.
Kinne, R. W., et al., Macrophages in rheumatoid arthritis. Arthritis Research & Therapy, 2000. 2(3): p. 189.
Haringman, J. J., et al., Synovial tissue macrophages: a sensitive biomarker for response to treatment in patients with rheumatoid arthritis. Ann Rheum Dis, 2005. 64(6): p. 834-8.
Li, S., et al., Nanomaterials Manipulate Macrophages for Rheumatoid Arthritis Treatment. Frontiers in Pharmacology, 2021. 12.
Deng, C., et al., Targeted apoptosis of macrophages and osteoclasts in arthritic joints is effective against advanced inflammatory arthritis. Nature Communications, 2021. 12(1): p. 2174.
Li, J., et al., Shield and sword nano-soldiers ameliorate rheumatoid arthritis by multi-stage manipulation of neutrophils. J Control Release, 2021. 335: p. 38-48.
Gouveia, V. M., et al., Non-Biologic Nanodelivery Therapies for Rheumatoid Arthritis. J Biomed Nanotechnol, 2015. 11(10): p. 1701-21.
Buckley, C. D., Why do leucocytes accumulate within chronically inflamed joints?Rheumatology, 2003. 42(12): p. 1433-1444.
Salmon, M., et al., Inhibition of T cell apoptosis in the rheumatoid synovium. J Clin Invest, 1997. 99(3): p. 439-46.
Cecchi, I., et al., Neutrophils: Novel key players in Rheumatoid Arthritis. Current and future therapeutic targets. Autoimmun Rev, 2018. 17(11): p. 1138-1149.
Mohammed, F. F., D. S. Smookler, and R. Khokha, Metalloproteinases, inflammation, and rheumatoid arthritis. Annals of the Rheumatic Diseases, 2003. 62(suppl 2): p. ii43-ii47.
Mangelsdorf, D. J., et al., Characterization of three RXR genes that mediate the action of 9-cis retinoic acid. Genes Dev, 1992. 6(3): p. 329-44.
Hoes, J. N., et al., Current view of glucocorticoid co-therapy with DMARDs in rheumatoid arthritis. Nature Reviews Rheumatology, 2010. 6(12): p. 693-702.
Schäcke, H., W. D. Döcke, and K. Asadullah, Mechanisms involved in the side effects of glucocorticoids. Pharmacol Ther, 2002. 96(1): p. 23-43.
Ren, H., et al., Role of Liposome Size, Surface Charge, and PEGylation on Rheumatoid Arthritis Targeting Therapy. ACS Appl Mater Interfaces, 2019. 11(22): p. 20304-20315.
Metselaar, J. M., et al., Liposomal targeting of glucocorticoids to synovial lining cells strongly increases therapeutic benefit in collagen type II arthritis. Ann Rheum Dis, 2004. 63(4): p. 348-53.
Rauchhaus, U., F. W. Schwaiger, and S. Panzner, Separating therapeutic efficacy from glucocorticoid side-effects in rodent arthritis using novel, liposomal delivery of dexamethasone phosphate: long-term suppression of arthritis facilitates interval treatment. Arthritis Research & Therapy, 2009. 11(6): p. R190.
Anderson, R., et al., Liposomal encapsulation enhances and prolongs the anti-inflammatory effects of water-soluble dexamethasone phosphate in experimental adjuvant arthritis. Arthritis Research & Therapy, 2010. 12(4): p. R147.
Meka, R. R., et al., Peptide-targeted liposomal delivery of dexamethasone for arthritis therapy. Nanomedicine (Lond), 2019. 14(11): p. 1455-1469.
Colino, C. I., J. M. Lanao, and C. Gutierrez-Millan, Targeting of Hepatic Macrophages by Therapeutic Nanoparticles. Frontiers in immunology, 2020. 11: p. 218-218.
Larson, N. and H. Ghandehari, Polymeric Conjugates for Drug Delivery. Chemistry of Materials, 2012. 24(5): p. 840-853.
Liu, X. M., et al., Syntheses of click PEG-dexamethasone conjugates for the treatment of rheumatoid arthritis. Biomacromolecules, 2010. 11(10): p. 2621-8.
Bhattacharya, M., et al., Release of functional dexamethasone by intracellular enzymes: A modular peptide-based strategy for ocular drug delivery. Journal of Controlled Release, 2020. 327: p. 584-594.
Quarta, C., et al., Molecular Integration of Incretin and Glucocorticoid Action Reverses Immunometabolic Dysfunction and Obesity. Cell Metab, 2017. 26(4): p. 620-632.e6.
Largee, B., et al., Smart Drug-Delivery Systems in the Treatment of Rheumatoid Arthritis: Current, Future Perspectives, in Smart Drug Delivery, A. Usama, H. Md. Faheem, and A. Juber, Editors. 2021, IntechOpen: Rijeka. p. Ch. 3.
Zhao, G., et al., Thermoresponsive polymeric dexamethasone prodrug for arthritis pain. Journal of Controlled Release, 2021. 339: p. 484-497.
Wang, X., et al., The Role of Reactive Oxygen Species in the Rheumatoid Arthritis-Associated Synovial Microenvironment. Antioxidants (Basel), 2022. 11(6).
Herold, M. J., K. G. McPherson, and H. M. Reichardt, Glucocorticoids in T cell apoptosis and function. Cell Mol Life Sci, 2006. 63(1): p. 60-72.
Gruver-Yates, A. L., M. A. Quinn, and J. A. Cidlowski, Analysis of glucocorticoid receptors and their apoptotic response to dexamethasone in male murine B cells during development. Endocrinology, 2014. 155(2): p. 463-74.
Callis, G. and D. Sterchi, Decalcification of Bone: Literature Review and Practical Study of Various Decalcifying Agents. Methods, and Their Effects on Bone Histology. Journal of Histotechnology, 1998. 21(1): p. 49-58.
Bolon, B., et al., Rodent preclinical models for developing novel antiarthritic molecules: comparative biology and preferred methods for evaluating efficacy. J Biomed Biotechnol, 2011. 2011: p.569068.
Wu, J., et al., TNF antagonist sensitizes synovial fibroblasts to ferroptotic cell death in collagen-induced arthritis mouse models. Nature Communications, 2022. 13(1): p. 676.

What is claimed:

1. A conjugate comprising:
(a) a pharmaceutical agent linked by a cleavable linker to polyethylene glycol (PEG); and
(b) a peptide linked to the PEG via a second linker,
wherein the PEG has a molecular weight between about 300 Da and about 20 kDa, and
wherein the peptide comprises SEQ ID NO: 1, SEQ ID NO: 49, SEQ ID NO: 50, or SEQ ID NO: 51.

2.-3. (canceled)

4. The conjugate of claim 1, wherein the peptide comprises at least one D-amino acid.

5.-6. (canceled)

7. The conjugate of claim 1, wherein the peptide is circular.

8.-9. (canceled)

10. The conjugate of claim 1, wherein the pharmaceutical agent is a glucocorticoid.

11. The conjugate of claim 10, wherein the glucocorticoid is prednisolone or dexamethasone.

12. The conjugate of claim 1, wherein the cleavable linker is a ROS-responsive linker, a pH-responsive linker, an amine-reactive linker, or an enzyme-responsive linker.

13. The conjugate of claim 12, wherein the cleavable linker is a thioacetic acid linker.

14. The conjugate of claim 13, wherein the thioacetic acid linker is 2,2′-thiodiacetic acid.

15.-16. (canceled)

17. The conjugate of claim 1, wherein the PEG is PEG2000, PEG5000, or PEG10000.

18. The conjugate of claim 1, wherein the second linker is a maleimide linker and/or comprises a maleimide linkage.

19. The conjugate of claim 1, wherein the pharmaceutical agent is a glucocorticoid, the cleavable linker is a thioacetic acid linker, and the PEG has a molecular weight between 2,000 and 10,000 Da.

20. A conjugate comprising the structure of formula 1:

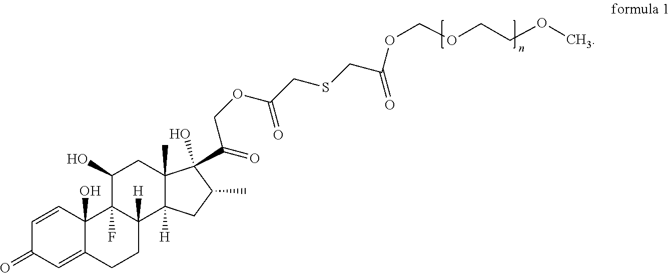

21. The conjugate of claim 1, wherein the peptide comprises SEQ ID NO: 1 and the second linker links a sulfur on a cysteine residue of the peptide to the PEG.

22. (canceled)

23. The conjugate of claim 1, further comprising a detectable label.

24. (canceled)

25. A pharmaceutical composition comprising the conjugate of claim 1 and a pharmaceutically acceptable carrier.

26. The pharmaceutical composition of claim 25, wherein the pharmaceutical composition is formulated for intravenous, subcutaneous, or oral administration, and/or wherein the pharmaceutical composition further comprises a hydrogel.

27. (canceled)

28. A method for reducing inflammation in a subject, the method comprising: administering a therapeutically effective amount of the pharmaceutical composition of claim 25 to the subject to reduce inflammation.

29. (canceled)

30. The method of claim 28, wherein the inflammation is associated with an inflammatory disease is-selected from the group consisting of acute lung injury (ALI), acute respiratory distress syndrome (ARDS), arthritis, lupus, eczema, chronic obstructive pulmonary disease (COPD), and obesity.

31. The method of claim 30, wherein (a) the inflammatory disease is acute lung injury (ALI) and the pharmaceutical agent is a glucocorticoid or (b) the inflammatory disease is arthritis, and the pharmaceutical agent is a dexamethasone.

32. (canceled)

33. The method of claim 28, wherein the conjugate accumulates in an inflamed tissue at higher levels than the pharmaceutical agent administered alone, wherein the inflamed tissue exhibits increased expression of retinoid X receptor beta (RXRB), and/or wherein the conjugate accumulates in healthy tissues at lower or equivalent levels to the pharmaceutical agent administered alone.

34. (canceled)

35. The method of claim 33, wherein the conjugate is administered intravenously, subcutaneously, or orally, and/or wherein the inflamed tissue is lung or a joint.

36.-36. (canceled)
